(19) United States
(12) Patent Application Publication
(10) Pub. No.: US 2026/0265379 A1
(43) Pub. Date: Sep. 10, 2026

(54) BISPECIFIC ANTIBODIES TARGETING BTN3A AND THE PD-1/PD-L1 INHIBITORY AXIS

(71) Applicant: Imcheck Therapeutics, Marseille (FR)

(72) Inventors: Jaime GUILLÉN, Marseille (FR); Magali COLAZET, Marseille (FR); Kieu-Suong LE, Marseille (FR); Aude DE GASSART, Marseille (FR)

(21) Appl. No.: 19/496,564

(22) PCT Filed: Jun. 21, 2024
(86) PCT No.: PCT/EP2024/067432

§ 371 Date: Dec. 23, 2025

(30) Foreign Application Priority Data
Jun. 23, 2023 (EP) 23181247.0

(51) Int. Cl. C07K 16/28; A61P 35/00

(52) U.S. Cl. CPC C07K 16/2827; A61P 35/00; C07K 2317/24; C07K 2317/31; C07K 2317/522; C07K 2317/524; C07K 2317/526; C07K 2317/55; C07K 2317/56; C07K 2317/622; C07K 2317/75; C07K 2317/92

(57) ABSTRACT

The invention relates to bispecific antibodies targeting the BTN3A receptor and blocking the PD-1/PD-L1 inhibitory axis. The invention also relates to bispecific antibodies capable of targeting immune cells, in particular γδ T cells, towards PD-L1-expressing tumor cells. The invention further relates to methods of treating cancer and pharmaceutical compositions comprising said bispecific antibodies.

### FIELD OF THE INVENTION

The invention relates to bispecific antibodies targeting the BTN3A receptor and blocking the PD-1/PD-L1 inhibitory axis. The invention also relates to bispecific antibodies capable of targeting immune cells, in particular γδ T cells, towards PD-L1-expressing tumor cells. The invention further relates to methods of treating cancer and pharmaceutical compositions comprising said bispecific antibodies.

### BACKGROUND

Programmed cell death protein-1 (PD-1) and its ligands, PD-L1 and PD-L2, are key factors in controlling immune responses (Nishimura et al. 1999; Freeman et al. 2000). Engagement of PD-1 by its ligands negatively regulates the T cell immune response, preventing damage to healthy tissues. However, tumors can leverage the PD-1 signaling pathway to evade immune-mediated destruction (Wei, Duffy, et Allison 2018). Blocking the interaction between PD-1 and its ligands activates T cell function and promotes tumor cell death (Hirano et al. 2005; Iwai, Terawaki, et Honjo 2005; Iwai et al. 2002; Strome et al. 2003). Moreover, tumor-infiltrating lymphocytes from patients with cancer often express high levels of PD-1 while tumor cells and other cells in the tumor tissue can upregulate PD-L1 expression (McDermott D F et al. 2013).

Antibodies targeting PD-1 or PD-L1 have revolutionized the treatment of a variety of cancer types. However, many patients still do not respond to checkpoint inhibitors and there is a need to identify alternative targets and approaches to improve therapeutic benefit for patients (Ribas et Wolchok 2018).

In many cases, the therapeutic efficacy of drugs targeting a single factor is usually reported to be ineffective (Uomo 2012). For example, the response rate of PD-1/PD-L1 monoclonal antibodies (mAbs) in solid cancers is only 20-30% (Jiang et al. 2020; Robert et al. 2015). To overcome these limitations, combination therapies and the use of bispecific antibodies (bsAbs) are of great interest.

Among attractive unconventional T cells, Vγ9Vδ2 T cells are emerging as promising effector cells that harbor strong cytolytic and pro-inflammatory activities and are associated with good prognosis in cancer patients (Gentles et al. 2015; Holtmeier et Kabelitz 2005; Tosolini et al. 2017). Gamma delta (γδ) T cells are an unconventional T cell subset with both innate and adaptive immune response features that play a key role in the immunosurveillance against malignancies and infections (Holtmeier et Kabelitz 2005). In the blood of healthy human adults, Vγ9Vδ2 T cells constitute the majority of circulating γδ T cells (50 to 90%) (Kabelitz et al. 2020) and represent 1 to 5% of total blood lymphocytes (Pauza et al. 2018). Unlike conventional αβ T cells that represent the major T cell subset among circulating T cells and recognize MHC-restricted antigens, γδ T cells are activated in a non-MHC-restricted manner. More specifically, Vγ9Vδ2 T cell activation is triggered by the intracellular accumulation of phosphoantigens (pAg) overproduced in response to viral and bacterial infections, metabolic stress or genetic dysregulation during carcinogenesis. Vγ9Vδ2 T cell activation under these pathophysiologic contexts induces broad functional activities that include the production of cytokines and chemokines, the cytolysis of infected or transformed target cells and interactions with other cells including epithelial cells, monocytes, dendritic cells (DC), neutrophils and B cells (Blazquez et al. 2018). With their potent anti-tumor activity (Holtmeier et Kabelitz 2005) and the association between their infiltration into malignant tissues and positive prognosis (Gentles et al. 2015; Tosolini et al. 2017), Vγ9Vδ2 T cells represent an attractive target for cancer immunotherapy.

A number of Vγ9Vδ2 T cell-based immuno-oncology therapeutic approaches have been explored in a variety of tumors in recent years using either in vivo activation of Vγ9Vδ2 T cells with amino-bisphosphonates (ABP) such as Zoledronate or synthetic pAg (i.e. BrHPP) in combination with IL-2, or adoptive transfer of autologous or allogeneic Vγ9Vδ2 T cells to patients following in vitro/ex vivo expansion (Kabelitz et al. 2020). While these Vγ9Vδ2 T cell-based therapies appear to be safe, clinical responses obtained were variable among patients (Kabelitz et al. 2020), suggesting the need to better understand the underlying mechanisms regulating Vγ9Vδ2 T responses in the tumor microenvironment to develop more effective immunotherapies. In tumor immunosurveillance, Vγ9Vδ2 T cell antitumoral activity is triggered by TCR-mediated recognition of pAg which is molecularly dependent on transmembrane butyrophilin (BTN) molecules BTN3A1 and BTN2A1 (Kabelitz et al. 2020).

BTN3A (also known as CD277) proteins belong to the BTN family and 3 isoforms have been described in humans: BTN3A1, BTN3A2 and BTN3A3. BTN3A isoforms are widely expressed in human immune cells and tissues and are mandatory for pAg sensing by Vγ9Vδ2 T cells (Arnett et Viney 2014; Blazquez et al. 2018; Rhodes, Reith, et Trowsdale 2016). Recently, Payne et al. have also shown that BTN3A1 inhibits tumor-reactive γβ TCR activation and that antibodies targeting BTN3A1 can prevent γβ T cell inhibition and simultaneously activate Vγ9Vδ2 T cells (Payne et al. 2020). The applicant has developed ICT01, a humanized mAb (human IgG1 with Fc-reduced effector functions) that recognizes all three BTN3A isoforms and possesses an agonist activity on Vγ9Vδ2 T cell functions. Therefore, said antibody represents a novel first-in-class immunotherapeutic agent for the treatment of solid and hematologic cancers. The patent applications WO2012/080351 and WO2020/025703 refer to various antibodies against BTN3A developed by ImCheck Therapeutics which can activate the cytokine production, proliferation and cytolytic function of Vγ9Vδ2 T cells.

Besides the Vγ9Vδ2-TCR engagement, activation of Vγ9Vδ2 T cells is also tightly regulated by several surface receptors including immune checkpoint receptors (ICRs) (Xiang et Tu 2017). Like for γβ T cells, increase of circulating and/or tumor infiltrating γδ T cells expressing ICRs was detected in many cancers (Girard et al. 2019; Guo et al. 2020; Hoeres et al. 2019; Jin et al. 2022; Li et al. 2020; Wu et al. 2020; 2019) and high proportion of γδ T cells expressing ICRs was associated to poor prognosis in cancer patients (Girard et al. 2019; Jin et al. 2022). Currently, there is no single molecule designed that is able to simultaneously activate Vγ9Vδ2 T cells through BTN3A targeting and block ICRs signaling. There is also no single molecule, to date, that has been proven to simultaneously activate Vγ9Vδ2 T cells through BTN3A targeting and bind on a tumor-associated antigen.

### SUMMARY OF THE INVENTION

The present inventors have hypothesized that anti-tumor activity of γδ T cells might be limited by ICRs signaling and therefore combining therapeutic checkpoint blockade with a γδ T cell stimulatory therapy could be more efficient and beneficial for cancer treatment. To this end, the inventors have developed therapeutic molecules that simultaneously activate Vγ9Vδ2 T cells and block the inhibitory axis PD-1/PD-L1. The bispecific antibodies developed by the inventors activate Vγ9Vδ2 T cells by targeting BTN3A and inhibit the PD-1/PD-L1 axis by targeting either PD-1 or PD-L1. PD-L1 is upregulated on a wide range of tumor cells which allows to exploit this target as tumor-associated antigen. The anti-BTN3A/anti-PD-L1 bispecific antibodies of the present disclosure can also be used to redirect a T cell activity towards a PD-L1-expressing tumor.

Accordingly, in one aspect, the invention relates to a bispecific antibody comprising at least one first antigen-binding moiety that specifically binds to BTN3A and at least one second antigen-binding moiety that specifically binds to an antigen selected from PD-1 and PD-L1.

In one embodiment, the first and/or second antigen-binding moieties are selected from an immunoglobulin molecule and an antigen-binding fragment selected from a Fab fragment, a Fab′ fragment, a F(ab′) 2 fragment, a scFab fragment, a single domain antibody or a Fv fragment such as a scFv fragment.

In one embodiment, one of the first and second antigen-binding moieties is an IgG molecule and the other of the first and second antigen-binding moieties is an antigen-binding fragment linked to the IgG molecule, wherein the antigen-binding fragment is selected from a Fab fragment, a Fab′ fragment, a F(ab′) 2 fragment, a scFab fragment, a single domain antibody or a Fv fragment such as a scFv fragment. Preferably, the IgG molecule is the first antigen-binding moiety that binds to BTN3A and the antigen-binding fragment is the second antigen-binding moiety that binds to PD-1 or PD-L1.

In one embodiment, the antigen-binding fragment linked to the IgG molecule is an scFv fragment.

In some embodiments of the bispecific antibody:
(i) the scFv fragment is linked to the C-terminal end of a heavy chain of the IgG molecule;
(ii) the scFv fragment is linked to the N-terminal end of a heavy chain of the IgG molecule;
(iii) the scFv fragment is linked to the C-terminal end of a light chain of the IgG molecule; and/or
(iv) the scFv fragment is linked to the N-terminal end of a light chain of the IgG molecule.

In some embodiments, the bispecific antibody comprises:
(i) two heavy chains having the formula [scFv-linker-IgG(H)] and two light chains having the formula [IgG(L)];
(ii) two heavy chains having the formula [IgG(H)-linker-scFv] and two light chains having the formula [IgG(L)];
(iii) two heavy chains having the formula [IgG(H)] and two light chains having the formula [scFv-linker-IgG(L)];
(iv) two heavy chains having the formula [IgG(H)] and two light chains having the formula [IgG(L)-linker-scFv];
(v) one heavy chain having the formula [scFv-linker-IgG(H)], one heavy chain having the formula [IgG(H)] and two light chains having the formula [IgG(L)];
(vi) one heavy chain having the formula [IgG(H)-linker-scFv], one heavy chain having the formula [IgG(H)] and two light chains having the formula [IgG(L)];
(vii) two heavy chains having the formula [IgG(H)], one light chain having the formula [scFv-linker-IgG(L)] and one light chain having the formula [IgG(L)];
(viii) two heavy chains having the formula [IgG(H)], one light chain having the formula [IgG(L)-linker-scFv] and one light chain having the formula [IgG(L)];
(ix) one heavy chain having the formula [IgG(H)], one heavy chain having the formula [scFv-linker-IgG Fc] and one light chain having the formula [IgG(L)];
(x) one heavy chain having the formula [IgG(H)-linker-scFv], one heavy chain having the formula [IgG Fc-linker-scFv] and one light chain having the formula [IgG(L)],
wherein IgG(H) and IgG(L) are, respectively, the heavy chain and light chain of the IgG molecule that specifically binds to BTN3A or PD-1 or PD-L1, in particular BTN3A, wherein IgG Fc is an IgG Fc region, and wherein scFv is the scFv fragment that specifically binds to BTN3A or PD-1 or PD-L1, in particular PD-1 or PD-L1, wherein, where more than one scFv is present, said scFv are identical or different and, where more than one linker is present, said linkers are identical or different.

In one embodiment, the antigen-binding fragment linked to the IgG molecule is an scFab fragment.

In some embodiments of the bispecific antibody:
(i) the scFab fragment is linked to the C-terminal end of a heavy chain of the IgG molecule;
(ii) the scFab fragment is linked to the N-terminal end of a heavy chain of the IgG molecule;
(iii) the scFab fragment is linked to the C-terminal end of a light chain of the IgG molecule; or
(iv) the scFab fragment is linked to the N-terminal end of a light chain of the IgG molecule.

In some embodiments, the bispecific antibody comprises:
(i) two heavy chains having the formula [scFab-linker-IgG(H)] and two light chains having the formula [IgG(L)];
(ii) two heavy chains having the formula [IgG(H)-linker-scFab] and two light chains having the formula [IgG(L)];
(iii) two heavy chains having the formula [IgG(H)] and two light chains having the formula [IgG(L)-linker-scFab]; or
(iv) two heavy chains having the formula [IgG(H)] and two light chains having the formula [scFab-linker-IgG(L)];
wherein IgG(H) and IgG(L) are, respectively, the heavy chain and light chain of the IgG molecule that specifically binds to BTN3A or PD-1 or PD-L1, preferably BTN3A, and wherein scFab is the scFab fragment that specifically binds to BTN3A or PD-1 or PD-L1, preferably PD-1 or PD-L1, wherein, where more than one scFab is present, said scFab are identical or different and, where more than one linker is present, said linkers are identical or different.

In one embodiment, the linker comprises an amino acid sequence selected from the sequences defined in SEQ ID NOs: 29 to 42.

In one embodiment, the Fc fragment of the immunoglobulin molecule is a silent Fc fragment, in particular a IgG1 Fc silent fragment.

In one embodiment, the first and second-antigen binding moieties are derived from human, chimeric or humanized antibodies.

In some embodiments, said bispecific antibody is capable of promoting the activation of PD-1 expressing immune cells, notably T cells, preferentially Vγ9Vδ2 T cells, in the presence of PD-L1-expressing target cells, such as cancer cells or antigen-presenting cells.

In some embodiments, said first antigen-binding moiety is selected from:
anti-BTN3A antibodies which comprise (a) a variable heavy chain (VH) polypeptide comprising an amino acid sequence that is at least about 95% identical to the amino acid sequence of SEQ ID NO: 1, and (b) a variable light chain (VL) polypeptide comprising an amino acid sequence that is at least about 95% identical to SEQ ID NO: 2, or anti-BTN3A antibodies which comprise (a) a variable heavy chain (VH) polypeptide comprising an amino acid sequence that is at least about 95% identical to the amino acid sequence of SEQ ID NO: 48, and (b) a variable light chain (VL) polypeptide comprising an amino acid sequence that is at least about 95% identical to SEQ ID NO: 49;
anti-BTN3A antibodies which comprise HCDRs1-3 of SEQ ID NO:3-5 and LCDRs1-3 of SEQ ID NO:6-8, or which comprise HCDR1 of SEQ ID NO:50, HCDR2 of SEQ ID NO: 51 or 56 to 59, HCDR3 of SEQ ID NO:52 and LCDR1 of SEQ ID NO: 53, 60 or 61, LCDR2 of SEQ ID NO: 54 and LCDR3 of SEQ ID NO:55;
antibodies which compete for binding to BTN3A with an anti-BTN3A antibody which comprises (a) a variable heavy chain (VH) polypeptide comprising an amino acid sequence of SEQ ID NO: 1, and (b) a variable light chain (VL) polypeptide comprising an amino acid sequence of SEQ ID NO: 2, or antibodies which compete for binding to BTN3A with an anti-BTN3A antibody which comprises (a) a variable heavy chain (VH) polypeptide comprising an amino acid sequence of SEQ ID NO: 48, and (b) a variable light chain (VL) polypeptide comprising an amino acid sequence of SEQ ID NO: 49; and
anti-BTN3A antibodies which compete for binding with an antibody selected from mAb 20.1 as produced by the hybridoma deposited at the CNCM under deposit number I-4401 or mAb 7.2 as produced by the hybridoma deposited at the CNCM under deposit number I-4402, or an antigen-binding fragment of said anti-BTN3A antibodies.

In some embodiments, said second binding domain binds specifically to PD-1 and is selected from:
anti-PD-1 antibodies which comprise (a) a variable heavy chain (VH) polypeptide comprising an amino acid sequence that is at least about 95% identical, in particular 100% identical, to the amino acid sequence of SEQ ID NO: 9, and (b) a variable light chain (VL) polypeptide comprising an amino acid sequence that is at least about 95% identical, in particular 100% identical, to the amino acid sequence of SEQ ID NO: 10;
anti-PD-1 antibodies which comprise a variable heavy chain (VH) polypeptide and a variable light chain (VL) polypeptide comprising an amino acid sequence that is at least about 95% identical, in particular 100% identical, to the amino acid sequence of the VH and VL chains of an anti-PD-1 antibody selected from pembrolizumab, nivolumab, dostarlimab, retifanlimab, spartalizumab, MEDI-0680, cemiplimab, pidilizumab, pucotenlimab, zimberelimab, prolgolimab, penpulimab, toripalimab, tislelizumab, camrelizumab, AM-0001, STI-1110, balstilimab, sintilimab and SG001, preferably selected from pembrolizumab, nivolumab, cemiplimab, dostarlimab and retifanlimab;
anti-PD-1 antibodies which comprise HCDRs1-3 of SEQ ID NO: 11-13 and LCDRs1-3 of SEQ ID NO: 14-16;
anti-PD-1 antibodies which comprise HCDRs1-3 and LCDRs1-3 of an anti-PD-1 antibody selected from pembrolizumab, nivolumab, dostarlimab, retifanlimab, spartalizumab, MEDI-0680, cemiplimab, pidilizumab, pucotenlimab, zimberelimab, prolgolimab, penpulimab, toripalimab, tislelizumab, camrelizumab, AM-0001, STI-1110, balstilimab, sintilimab and SG001, preferably selected from pembrolizumab, nivolumab, cemiplimab, dostarlimab and retifanlimab;
anti-PD-1 antibodies which compete with an antibody selected from pembrolizumab, nivolumab, dostarlimab, retifanlimab, spartalizumab, MEDI-0680, cemiplimab, pidilizumab, pucotenlimab, zimberelimab, prolgolimab, penpulimab, toripalimab, tislelizumab, camrelizumab, AM-0001, STI-1110, balstilimab, sintilimab and SG001, preferably selected from pembrolizumab, nivolumab, cemiplimab, dostarlimab and retifanlimab for binding to PD-1;
or an antigen-binding fragment of said antibodies.

In some embodiments, said second binding domain binds specifically to PD-L1 and is selected from:
anti-PD-L1 antibodies which comprise (a) a variable heavy chain (VH) polypeptide comprising an amino acid sequence that is at least about 95% identical, in particular 100% identical to the amino acid sequence of SEQ ID NO: 17, and (b) a variable light chain (VL) polypeptide comprising an amino acid sequence that is at least about 95% identical, in particular 100% identical, to the amino acid sequence of SEQ ID NO: 18;
anti-PD-L1 antibodies which comprise a variable heavy chain (VH) polypeptide and a variable light chain (VL) polypeptide, respectively comprising an amino acid sequence that is at least about 95% identical, in particular 100% identical, to the amino acid sequence of the VH and VL chains of an anti-PD-L1 antibody selected from durvalumab, atezolizumab, avelumab, BMS-936559, LY3300054, pacmilimab, FAZ053, envafolimab, and MDX-1105;
anti-PD-L1 antibodies which comprise HCDRs1-3 and LCDRs1-3 of an anti-PD-L1 antibody selected from durvalumab, atezolizumab, avelumab, BMS-936559, LY3300054, pacmilimab, FAZ053, envafolimab, and MDX-1105;
anti-PD-L1 antibodies which comprise HCDRs1-3 of SEQ ID NO: 19-21 and LCDRs1-3 of SEQ ID NO:22-24;
anti-PD-L1 antibodies which compete with an anti-PD-L1 antibody selected from durvalumab, atezolizumab, avelumab, BMS-936559, LY3300054, pacmilimab, FAZ053, envafolimab, and MDX-1105 for binding to PD-L1;
or an antigen-binding fragment of said antibodies.

In some embodiments,
(a) said second binding moiety binds specifically to PD-1 and is selected from pembrolizumab, nivolumab, dostarlimab, retifanlimab, spartalizumab, MEDI-0680, cemiplimab, pidilizumab, pucotenlimab, zimberelimab, prolgolimab, penpulimab, toripalimab, tislelizumab, camrelizumab, AM-0001, STI-1110, balstilimab, sintilimab and SG001, preferably from pembrolizumab, nivolumab, cemiplimab, dostarlimab and retifanlimab, or an antigen-binding fragment thereof; or
(b) said second binding moiety binds specifically to PD-L1 and is selected from durvalumab, atezolizumab, avelumab, BMS-936559, LY3300054, pacmilimab, FAZ053, envafolimab, and MDX-1105, preferably from atezolizumab, durvalumab or avelumab, or an antigen-binding fragment thereof.

In some embodiments, said bispecific antibody comprises an IgG molecule, wherein said IgG molecule comprises:
CH1, CH2 and CH3 domains comprising the amino acid sequence set forth in SEQ ID NO:26, or
CH2 and CH3 domains comprising the amino acid sequence set forth in SEQ ID NO:27 or 28;
or an amino acid sequence with at least 95% sequence identity with said amino acid sequences.

In some embodiments, said bispecific antibody comprises an IgG molecule, wherein said IgG molecule comprises a CL domain comprising the amino acid sequence set forth in SEQ ID NO: 25 or an amino acid sequence with at least 95% sequence identity with said amino acid sequences.

The invention further relates to a polynucleotide encoding a light chain and/or a heavy chain of the bispecific antibody according to the invention. Suitable polynucleotides encoding the light and/or heavy chains can be designed by assembly of sequences encoding the domains of a bispecific antibody of the invention, as described herein. In some embodiments, the polynucleotide comprises a nucleotide sequence encoding an anti-BTN3A VH domain, wherein said sequence is selected from SEQ ID NO:46 and sequences with at least 80%, preferably 85%, still preferably 90%, more preferably 95% and most preferably 99% sequence identity with SEQ ID NO:46. In some embodiments, the polynucleotide comprises a nucleotide sequence encoding an anti-BTN3A VL domain, wherein said sequence is selected from SEQ ID NO: 47 or sequences with at least 80%, preferably 85%, still preferably 90%, more preferably 95% and most preferably 99% sequence identity with SEQ ID NO:47.

Further polynucleotide sequences useful for the invention are disclosed in the various references cited in the present disclosure in connection, e.g. with anti-PD-1 antibodies (e.g. WO2008/156712), anti-PD-L1 antibodies (e.g. WO2011066389) and anti-BTN3A antibodies (e.g. WO2020/025703, WO2012/080769, WO2012/080351, WO2020/136218, WO 2020/033923, WO 2020/033926, WO2023/161457 and Dai et al, 2024).

The invention further relates to a vector, particularly an expression vector, comprising the polynucleotide according to the invention.

The invention further relates to a prokaryotic or eukaryotic host cell comprising the polynucleotide according to the invention or the vector according to the invention.

The invention further relates to a method of producing the bispecific antibody according to the invention, comprising the steps of a) transforming a host cell with vectors comprising one or more polynucleotides encoding said bispecific antibody, b) culturing the host cell under conditions suitable for the expression of the bispecific antibody and c) recovering the bispecific antibody from the culture.

The invention also relates to a pharmaceutical composition comprising the bispecific antibody according to the invention and/or the polynucleotide according to the invention, and a pharmaceutically acceptable carrier.

The invention also relates to a bispecific antibody according to the invention and/or the polynucleotide according to the invention for use in therapy, in particular for use in the treatment and/or prevention of a cancer.

In one embodiment, said bispecific antibody is used in combination with one or more additional therapeutic agents.

### DETAILED DESCRIPTION OF THE INVENTION

### Definitions

In order that the present disclosure be more readily understood, certain terms are first defined. Additional definitions are set forth throughout the detailed description.

Various embodiments as described in the present detailed description can be combined according to the present invention unless clearly specified.

As used herein, the term “protein” refers to any organic compound made of amino acids arranged in one or more linear chains (also referred as “polypeptide chains”) and folded into a globular form. The amino acids in such polypeptide chain are joined together by the peptide bonds between the carboxyl and amino groups of adjacent amino acid residues. The term “protein” further includes, without limitation, peptides, single chain polypeptide or any complex proteins consisting primarily of two or more chains of amino acids. It further includes, without limitation, glycoproteins or other known post-translational modifications. It further includes known natural or artificial chemical modifications of natural proteins, such as without limitation, glycoengineering, pegylation, hesylation and the like, incorporation of non-natural amino acids, amino acid modification for chemical conjugation or any other molecule, etc. As used herein, the term “amino acid” refers to either natural and/or unnatural or synthetic amino acids, including both D or L optical isomers, and amino acid analogues and peptidomimetics.

The terms “polypeptide,” “peptide” and “protein” expressly include glycoproteins, as well as non-glycoproteins. In specific embodiments, the term “polypeptide” and “protein” refers to any polypeptide or protein that can be encoded by a gene and translated using cell expression system and DNA recombinant means, such as mammalian host cell expression system. It is to be noted that cell free expression systems may also be used to produce any of bispecific mAbs as herein disclosed. Typically, methods of cell-free expression of proteins or antibodies are already described (Stech et al. 2017).

The term “recombinant protein”, as used herein, includes proteins that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) fusion proteins isolated from a host cell transformed to express the corresponding protein, e.g., from a transfectoma, etc . . . .

The term “antibody” as used herein refers to immunoglobulin molecules and immunologically active portions of immunoglobulin molecules, i.e., molecules that contain an antigen binding site that immunospecifically binds to an antigen. As used herein the term “antibody” or “immunoglobulin” have the same meaning and are used equally in the present disclosure. The term “antibody” typically includes substantially intact antibody molecules, as well as chimeric antibodies, humanized antibodies, isolated human antibodies, single chain antibodies, bispecific antibodies, antibody heavy chains, antibody light chains, homodimers and heterodimers of antibody heavy and/or light chains.

In natural antibodies of rodents and primates, two heavy chains are linked to each other by disulfide bonds, and each heavy chain is linked to a light chain by a disulfide bond. There are two types of light chains, lambda (λ) and kappa (κ). There are five main heavy chain classes (or isotypes) which determine the functional activity of an antibody molecule: IgM, IgD, IgG, IgA and IgE. Each chain contains distinct sequence domains.

IgGs are the most abundant antibodies in the blood and are currently used as backbone for antibody therapeutics. An IgG antibody consists of heavy and light domains that connect to form chains. Light chains consist of two light domains, a variable domain (VL) and a constant domain (CL). Heavy chains consist of four heavy domains, a variable domain (VH) and three constant domains (CH1, CH2 and CH3, collectively referred to as CH). A light and heavy chain together form a pair, and two heavy-light chain pairs form an antibody. The region where the two pairs connect is called the hinge region. Endogenous IgGs have small variations in their hinge regions, resulting in IgG subtypes. The Fab fragment contains the variable fragments that form the binding sites. The Fv fragment is the N-terminal part of the Fab fragment of an immunoglobulin and consists of the variable portions of one light chain and one heavy chain. An antibody can be also divided into functional parts. The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding and binding to Fc receptors (FcR). The Fc region or antibody's tail is composed of two identical protein fragments derived from the second and third constant domains of the antibody's two heavy chains. The Fc region connected to the Fab region mediates the effector functions that lead to immune-mediated target-cell killing (Scott, Wolchok, & Old, 2012). The Fc region can also be recognized by a receptor called the neonatal receptor, which is involved in regulating the IgG serum levels and actively prolongs the biological half-life (Roopenian & Akilesh, 2007). This process is called neonatal recycling.

The specificity of the antibody resides in the structural complementarity between the antibody combining site and the antigenic determinant. Antibody combining sites are made up of residues that are primarily from the hypervariable or complementarity determining regions (CDRs). Occasionally, residues from non-hypervariable or framework regions (FR) can participate to the antibody binding site or influence the overall domain structure and hence the combining site. Complementarity Determining Regions or CDRs refer to amino acid sequences, which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDRs set from each of a heavy and a light chain V region. Framework Regions (FRs) refer to amino acid sequences interposed between CDRs. Accordingly, the variable regions of the light and heavy chains typically comprise 4 framework regions and 3 CDRs of the following sequence: FR1-CDR1-FR2-CDR2-FR3-CDR3-FR4.

The residues in antibody variable domains are conventionally numbered according to a system devised by Kabat et al. This system is set forth in Kabat et al., 1987, in Sequences of Proteins of Immunological Interest, US Department of Health and Human Services, NIH, USA (hereafter “Kabat et al.”). This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35 (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system.

The term “antigen-binding fragment” of an antibody (or simply “antibody fragment”), as used herein, refers to full length or one or more fragments of an antibody that retain the ability to specifically bind to an antigen. It has been shown that the antigen-binding function of an antibody can be performed by fragments of a full-length antibody. Examples of binding fragments encompassed within the term “antigen-binding fragment” of an antibody include a Fab fragment, a monovalent fragment consisting of the VL, VH, CL and CH1 domains; a F (ab′) 2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., 1989 Nature 341:544-546), which consists of a VH domain, or any fusion proteins comprising such antigen-binding fragments. Furthermore, although the two domains of the Fv fragment, VL and VH, are coded for by separate genes, they can be joined, using recombinant methods, by a synthetic linker that enables them to be made as a single chain protein in which the VL and VH regions pair to form monovalent molecules (known as single chain Fv (scFv); see e.g., Bird et al., 1988 Science 242:423-426; and Huston et al., 1988 Proc. Natl. Acad. Sci. 85:5879-5883). Such single chain antibodies are also intended to be encompassed within the term “antigen-binding fragment” of an antibody. These antibody fragments are obtained using conventional techniques known to those of skill in the art, and the fragments are screened for utility in the same manner as are intact antibodies. The term “antigen-binding fragment” (of an antibody) as used herein also include single domain antibodies such as VHH or nanobodies™

The term “single-domain antibody” (sdAb) or nanobody® (tradename of Ablynx) has its general meaning in the art and refers to an antibody fragment with a molecular weight of only 12-15 kDa consisting of the single heavy chain variable domain of antibodies of the type that can be found in Camelid mammals and which are naturally devoid of light chains. Thus, in some embodiments, such single-domain antibodies can be VHHs (variable heavy homodimers). For a general description of these (single) domain antibodies, reference is also made to the prior art cited above, as well as to EP 0 368 684, Ward et al. (Nature 1989 Oct. 12; 341 (6242): 544-6), Holt et al, Trends Biotechnol, 2003, 21 (1 I): 484-490; and WO 06/030220, WO 06/003388. The amino acid sequence and structure of a single-domain antibody can be considered to be comprised of four framework regions or “FRs” which are referred to in the art and herein as “Framework region 1” or “FR1”; as “Framework region 2” or “FR2”; as “Framework region 3” or “FR3”; and as “Framework region 4” or “FR4” respectively; which framework regions are interrupted by three complementary determining regions or “CDRs”, which are referred to in the art as “Complementarity Determining Region 1” or “CDR1”; as “Complementarity Determining Region 2” or “CDR2” and as “Complementarity Determining Region 3” or “CDR3”, respectively. Accordingly, the single-domain antibody can be defined as an amino acid sequence with the general structure: FR1-CDR1-FR2-CDR2-FR3-CDR3-FR4 in which FR1 to FR4 refer to framework regions 1 to 4 respectively, and in which CDR1 to CDR3 refer to the Complementarity Determining Regions 1 to 3.

In specific embodiments of the present disclosure an antigen binding fragment is a single domain antibody or a scFv.

As used herein, the term “IgG Fc region” is used to define the C-terminal region of an immunoglobulin heavy chain, including native sequence Fc region and variant Fc regions. The human IgG heavy chain Fc region is generally defined as comprising the amino acid residue from position C226 or from P230, to the carboxyl-terminus of the IgG antibody of human gamma heavy chain or its counterpart sequence in other types of antibody heavy chains (e.g., α, δ, ε and μ for human antibodies), or a naturally occurring allotype thereof. The numbering of residues in the Fc region is that of the Kabat numbering system.

The C-terminal lysine (residue K447) of the Fc region may be removed, for example, during production or purification of the antibody. Accordingly, a composition of antibodies of the invention may comprise antibody populations with all K447 residues removed, antibody populations with no K447 residues removed, and antibody populations having a mixture of antibodies with and without the K447 residue.

Unless otherwise specified, the commonly accepted Kabat amino acid numbering for immunoglobulins is used throughout this disclosure (see Kabat et al. (1991) Sequences of Protein of Immunological Interest, 5th ed., United States Public Health Service, National Institute of Health, Bethesda, MD).

As used herein, the term “binding” in the context of the binding of an antibody to a predetermined antigen or epitope, notably BTN3A, PD-1 or PD-L1, means typically a binding with an affinity corresponding to a K_{D} of about 10⁻⁷ M or less, such as about 10⁻⁸ M or less, such as about 10⁻⁹ M or less, about 10⁻¹⁰ M or less, or about 10⁻¹¹ M or even less when determined by for instance surface plasmon resonance (SPR) technology in a BIAcore 3000 or a Carterra LSA Platform instrument using typically a soluble form of the antigen as the analyte and the antibody as the ligand.

The term “K_{assoc}” or “Kₐ”, as used herein, is intended to refer to the association rate of a particular antibody-antigen interaction, whereas the term “K_{dis}” or “K_{d},” as used herein, is intended to refer to the dissociation rate of a particular antibody-antigen interaction.

The term “K_{D}”, as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art. Methods for determining the K_{D} of a protein or an antibody include the use of SPR, for example the use of a biosensor system such as a BIAcore® system, or of Luminex assay, Octet® (Abdiche et al. 2008) (see also for detailed information regarding affinity assessment Rich R L, Day Y S, Morton T A, Myszka D G. High-resolution and high-throughput protocols for measuring drug/human serum albumin interactions using BIAcore®. Anal Biochem. 2001 Sep. 15; 296(2):197-207), or Carterra LSA Platform. The Octet® platform is based on bio-layer interferometry (BLI) technology. The principle of BLI technology is based on the optical interference pattern of white light reflected from two surfaces—a layer of immobilized protein and an internal reference layer. The binding between a ligand immobilized on the biosensor tip surface and an analyte in solution produces an increase in optical thickness at the biosensor tip, which results in a shift in the interference pattern measured in nanometers. The wavelength shift (Δλ) is a direct measure of the change in optical thickness of the biological layer, when this shift is measured over a period of time and its magnitude plotted as a function of time, a classic association/dissociation curve is obtained. This interaction is measured in real-time, allowing to monitor binding specificity, association rate and dissociation rate, and concentration. Affinity measurements are typically performed at 25° C.

“Selective binding” typically means that the antibody binds more strongly to a target, such as an epitope, for which it is specific as compared to the binding to another target. The antibody binds more strongly to a first target as compared to a second target if its affinity for the first target is higher than its affinity for the second target. Typically, an antibody binds more strongly to a first target as compared to a second target if it binds to the first target with a dissociation constant (Kd), or an EC₅₀, that is lower than the dissociation constant, or the EC₅₀, for the second target. Most specifically the agent does not bind at all to the second target to a relevant extent.

Typically, an antibody binds to the specific antigen with an affinity corresponding to a K_{D} that is at least ten-fold lower, such as at least 100-fold lower, for instance at least 1,000-fold lower, such as at least 10,000-fold lower, for instance at least 100,000-fold lower than its K_{D} for binding to a non-specific antigen (e.g., BSA, casein), which is not identical or closely related to the predetermined antigen. When the K_{D} of the antibody is very low (that is, the antibody has a high affinity), then the K_{D} with which it binds the antigen is typically at least 10,000-fold lower than its K_{D} for a non-specific antigen. Binding specificity may also be assessed by determination of EC₅₀. The phrases “an antibody recognizing an antigen” and “an antibody having specificity for an antigen” are used interchangeably herein with the term “an antibody which binds specifically to an antigen”.

Selectivity of an antibody as herein disclosed may be tested using cross-reactivity assays to other closely related proteins compared with the intended target protein. When such cross-reactivity cannot be detected, while giving a strong signal of the intended target at the same time and at the same antibody dilution, the antibody is typically deemed selective. An antibody that “cross-reacts with an antigen” is intended to refer to an antibody that binds that antigen with a K_{D} of 10 nM or less, 1 nM or less, or 100 μM or less. An antibody that “does not cross-react with a particular antigen” is intended to refer to an antibody that binds to that antigen, with a K_{D} of 100 nM or greater, or a K_{D} of 1 mM or greater, or a K_{D} of 10 mM or greater, said affinity being measured for example using similar SPR measurements, as disclosed in the Examples. In certain embodiments, such antibodies that do not cross-react with the antigen exhibit essentially undetectable binding against these proteins in standard binding assays.

As used herein, the term “subject” or “patient” includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc.

### Structural Variants of an Antibody, or Fragments Thereof:

Suitable structural variants of an antibody according to the present disclosure encompasses, without limitation, the variants as described below.

“Chimeric antibodies” are molecules made up of domains from different species. The term “chimeric antibody” refers to a monoclonal antibody, which comprises a VH domain and a VL domain of an antibody derived from a non-human animal, and CH domains and a CL domain from a human antibody. As the non-human animal, any animal such as camelids, mouse, rat, hamster, rabbit or the like can be used.

“Humanized antibody” as used herein, refers broadly to include antibodies made by a non-human cell having variable and constant regions which have been altered to more closely resemble antibodies that would be made by a human cell, for example, by altering the non-human antibody amino acid sequence to incorporate amino acids found in human germline immunoglobulin sequences. The humanized antibodies may include amino acid residues not encoded by human germline immunoglobulin sequences (e.g., mutations introduced by random or site-specific mutagenesis in vitro or by somatic mutation in vivo), for example in the CDRs. The antibodies as used herein may include amino acid residues not encoded by human sequences (e.g., mutations introduced by random or site-specific mutagenesis in vitro or by somatic mutation in vivo). More specifically, the term “humanized antibody”, as used herein, may include antibodies in which CDR sequences derived from the germline of another mammalian species, such as a mouse, have been grafted onto human framework sequences. In specific embodiments, the term «humanized antibody» includes antibodies which have the 6 CDRs of a murine antibody, but humanized framework and human constant regions. In some embodiments, the term “humanized antibody” includes antibodies that comprise a silent variant of Fc IgG region.

Suitable variants typically exhibit at least about 80% of identity to the parent peptide. According to the present disclosure a first amino acid sequence having at least 80% of identity with a second amino acid sequence means that the first sequence has 80; 81; 82; 83; 84; 85; 86; 87; 88; 89; 90; 91; 92; 93; 94; 95; 96; 97; 98; 99; or 100% of identity with the second amino acid sequence.

Typically, variants of a reference antibody include:
antibodies or fragment thereof having at least 80%, notably at least, 85, 90, 95, 96, 97, 98, 99 or 100% identity with the VH and VL regions of the reference antibody or fragment thereof.
antibodies or fragment thereof having at least 80%, notably at least, 85, 90, 95, 96, 97, 98, 99 or 100% identity with light chain and/or heavy chain of the reference antibody or fragment thereof.

The percent identity between two amino acid sequences can be determined using the Needleman and Wunsch algorithm (NEEDLEMAN, and Wunsch). The percent identity between two nucleotide or amino acid sequences may also be determined using for example algorithms such as EMBOSS Needle (pairwise alignment; available at www.ebi.ac.uk). For example, EMBOSS Needle may be used with a BLOSUM62 matrix, a “gap open penalty” of 10, a “gap extend penalty” of 0.5, a false “end gap penalty”, an “end gap open penalty” of 10 and an “end gap extend penalty” of 0.5. In general, the “percent identity” is a function of the number of matching positions divided by the number of positions compared and multiplied by 100. For instance, if 6 out of 10 sequence positions are identical between the two compared sequences after alignment, then the identity is 60%. The % identity is typically determined over the whole length of the query sequence on which the analysis is performed. Two molecules having the same primary amino acid sequence or nucleic acid sequence are identical irrespective of any chemical and/or biological modification.

Other antibody variants as disclosed herein include those having amino acids that have been mutated by amino acid deletion, insertion or substitution, yet have at least 60, 70, 80, 90, 95, 96, 97, 98, 99 or 100% identity in the CDR regions as compared with the CDR regions of the reference mAb. Typically, as per the present disclosure, antibodies may have between 1, 2, 3 or 4 amino acid variations (including deletion, insertion or substitution) in one or more CDRs, as compared to the CDR sequences of the reference antibody. In some embodiments, the sequences of CDR variants may differ from the sequence of the CDRs of the parent antibody sequences through mostly conservative substitutions; for instance, at least 10, such as at least 9, 8, 7, 6, 5, 4, 3, 2 or 1 of the substitutions in the variant are conservative amino acid residue replacements. In the context of the present disclosure, conservative substitutions may be defined by substitutions within the classes of amino acids reflected as follows:
Aliphatic residues I, L, V, and M
Cycloalkenyl-associated residues F, H, W, and Y
Hydrophobic residues A, C, F, G, H, I, L, M, R, T, V, W, and Y
Negatively charged residues D and E
Polar residues C, D, E, H, K, N, Q, R, S, and T
Positively charged residues H, K, and R
Small residues A, C, D, G, N, P, S, T, and V
Very small residues A, G, and S
Residues involved in turn A, C, D, E, G, H, K, N, Q, R, S, P, and formation T
Flexible residues Q, T, K, S, G, P, D, E, and R

More conservative substitution groupings include valine-leucine-isoleucine, phenylalanine-tyrosine, lysine-arginine, alanine-valine, and asparagine-glutamine. Conservation in terms of hydropathic/hydrophilic properties and residue weight/size also is substantially retained in a variant CDR as compared to a CDR of the reference antibody. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). The retention of similar residues may also or alternatively be measured by a similarity score, as determined by use of a BLAST program (e.g., BLAST 2.2.8 available through the NCBI using standard settings BLOSUM62, Open Gap=11 and Extended Gap=1).

In some embodiments, the variants have the 6 CDR regions 100% identical to the 6 corresponding CDR regions of the reference mAb, and include mutant amino acid sequences wherein no more than 1, 2, 3, 4 or 5 amino acids have been mutated by amino acid deletion, insertion or substitution in the FR1, FR2, FR3 and FR4 regions when compared with the corresponding framework regions of the corresponding reference antibody.

Structural variant antibodies with mutant amino acid sequences can for example also be obtained by mutagenesis (e.g., site-directed or PCR-mediated mutagenesis) of the coding nucleic acid molecules, or by affinity maturation, followed by testing of the altered antibody for retained function(s) (e.g., specificity, affinity and other functional properties) as compared using appropriate functional assays (see below and the examples).

### Function and Structure of the Bispecific Antibodies

The term “bispecific” as used herein means that the polypeptide is capable of specifically binding at least two target entities. Using bispecific antibodies enables novel and unique mechanisms of actions, such as specific targeting of T cells to the tumor cells or pathogens, or engagement of immune cells to tumor cells (Chames & Baty, 2009; Fan, Wang, Hao, & Li, 2015).

The bispecific antibodies as disclosed herein comprise as a binding specificity at least one antibody, or an antibody fragment thereof. Binding of the bispecific molecules to their specific targets can be confirmed by, for example, enzyme-linked immunosorbent assay (ELISA), radioimmunoassay (REA), FACS analysis, bioassay (e.g., growth inhibition and apoptosis), or Western Blot assay, more specifically using flow cytometry assays or Octet™ platform (ForteBio), as notably illustrated in the examples herein. Each of these assays generally detects the presence of protein-antibody complexes of particular interest by employing a labelled reagent (e.g., an antibody) specific for the complex of interest.

Antibodies (i.e., monoclonal antibodies (mAb)) and antigen-binding fragments thereof, which can be employed in the bispecific antibody molecules disclosed herein are typically from murine, camelid, human, chimeric and humanized monoclonal antibodies.

The binding affinity of a bispecific antibody as per the present disclosure for its targets can typically be assessed through classical in cellulo binding assays, on cell lines genetically modified to express the targets of interest or genetically modified to extinct target expression (used as negative control).

In this context, in some embodiments, bispecific antibodies of the present disclosure exhibit one or more of the following features:
(i) bind to BTN3A with a K_{D} of 100 nM or less, preferably with a K_{D} of 10 nM or less, still preferably with a K_{D} of 1 nM or less, still more preferably with a K_{D} of 0.1 nM or less as measured by SPR;
(ii) cross-react to cynomolgus BTN3A with a K_{D} of 100 nM or less, preferably with a K_{D} of 10 nM or less, as measured by SPR;
(iii) bind to human PBMCs with an EC₅₀ of 100 μg/ml or below, preferably of 50 μg/ml or below, still preferably of 10 μg/ml or below, as measured in a flow cytometry assay;
(iv) induce the activation of γδ T cells, typically Vγ9Vδ2 T cells, in co-culture with BTN3A expressing cells, with an EC₅₀ below 5 μg/ml, preferably of 1 μg/ml or below, as measured in a degranulation assay and/or reporter assay, as described in Example 4;
(v) induce the killing of tumor cells (e.g. ovarian cancer cell line SKOV3) by γδ T cells, typically Vγ9Vδ2 T cells, with an EC₅₀ below 1 nM, in particular below 0.1 nM, more particularly below 0.01 nM, as determined in an in vitro killing assay (as described in Example 5);
(vi) induce an interferon-gamma production by T cells in a Mixed Lymphocyte Reaction (MLR) assay (as described in Example 7) at a level superior to the combination of monoclonal antibodies binding specifically, respectively, to the first antigen and second antigen, such as an anti-BTN3A mAb and an anti-PD-L1 mAb (e.g., ICT01 and Durvalumab).

In some embodiments, a bispecific antibody of the present disclosure binds to PD-1 or PD-L1 with a K_{D} of 100 nM or less, preferably with a K_{D} of 10 nM or less, still preferably with a K_{D} of 1 nM or less, still more preferably with a K_{D} of 0.1 nM or less as measured by SPR.

The bispecific antibodies as per the present disclosure induce T cell activation, preferentially γδ T cell activation, and notably Vγ9Vδ2 T cell activation. In particular, the bispecific antibodies as per the present disclosure prevent inhibition of T cell activation by the PD-1/PD-L1 signaling pathway.

Methods for determining a change in T cell activity are well-known and include, for example, killing assays, cytokine production measurement (e.g., IFN-γ or TNFα), T cell proliferation monitoring, T cell degranulation assessment, etc. Killing assays as well as degranulation assays are well-known in the field and are illustrated in the examples herein.

More specifically bispecific antibodies of the present invention may be capable of promoting the activation of immune cells, in particular PD-1-expressing T cells, notably PD-1-expressing Vγ9Vδ2 T cells in the presence of PD-L1-expressing target cells, such as cancer cells or antigen-presenting cells. The PD-1-expressing T cells, notably Vγ9Vδ2 T cells, and the PD-L1-expressing cells are for instance co-cultured.

Promoting activation of said T cells, notably Vγ9Vδ2 T cells, may include:
activating the expression of a gene having Vγ9Vδ2 TCR-dependent expression (detailed below);
activating the cytolytic function of activated γδ T cells, as measured typically by a degranulation assay (detailed below);
activating the killing properties of γδ T cells against a target cell, as assessed typically by killing assays (detailed below);
activating the production of cytokines (e.g., IFN-γ or TNFα) and/or cytolytic molecules (e.g., Granzyme, Perforin) by activated γδ T cells, and/or
increasing the proliferation of activated γδ T cells.

As used herein, by “activating the expression of a gene having Vγ9Vδ2 TCR-dependent expression”, it is meant that a detectable increase in the expression of a gene can be measured in Vγ9Vδ2 T cells, wherein the expression of this gene is dependent on the activation of the Vγ9Vδ2 TCR. The gene expression increase is observed in the presence of the tested antibody compared to the corresponding isotype control said human γδ T cells being co-cultured with a target cell line expressing BTN3A and PD-L1 (such as Raji) or phosphoantigens (pAg). The gene may be a reporter gene such as luciferase, under the control of a TCR-dependent promoter or transcription factor, e.g. NFAT. In some embodiments, a bispecific antibody activates the Vγ9Vδ2 TCR in coculture with target cells expressing BTN3A (such as cancer cells, e.g. Raji cell line), as measured in a Jurkat-Vγ9Vδ2 TCR MOP reporter assay with an EC₅₀ below 1 nM, in particular below 0.1 nM, more particularly below 0.01 nM, still more particularly below 0.001 nM, for instance between 0.001 nM and 20 nM, in particular between 0.001 nM and 5 nM or between 0.001 nM and 1 nM.

As used herein, by “activating the cytolytic functions of activated γδ T cells”, it is meant that a significant increase of the cytolytic functions of activated human γδ T cells, notably Vγ9Vδ2 T cells, is observed in the presence of the tested antibody compared to the corresponding isotype control, said human γδ T cells being co-cultured with a target cell line expressing BTN3A and PD-L1 (such as Daudi or SKOV3 cell lines) or phosphoantigens (pAg). Typically, cytolytic function enhancement of γδ T cells, notably Vγ9Vδ2 T cells, may be measured based on the expression level of CD107, a degranulation marker, at their cell surface. In some embodiments, a bispecific antibody of the invention increases the expression level of CD107 on γδ T cells, notably Vγ9Vδ2 T cells, in coculture with target cells expressing BTN3A (such as cancer cells, e.g. ovarian cancer cell line SKOV3) by at least 2-fold, notably at least 2.5-fold, at least 3-fold, at least 5-fold or at least 10-fold, in particular when assessed at a concentration of 0.01 nM or more, or 0.1 nM or more, or 1 nM or more.

Phorbol 12-myristate 13-acetate (PMA) with ionomycin treatment can typically be used as positive control for Vγ9Vδ2 T cell activation. In some embodiments, a bispecific antibody activates the cytolytic functions of γδ T cells against target cells expressing BTN3A and PD-L1 (such as cancer cells, notably such as Daudi or SKOV3 cell lines), as measured in a degranulation assay with an EC₅₀ below 50 nM, in particular below 20 nM, notably below 10 nM, more particularly below 5 nM, for example comprised between 0.05 and 20 nM, notably between 0.05 and 10 nM, between 0.05 and 5 nM or between 0.1 and 10 nM.

As used herein, by “activating the production of cytokines and/or cytolytic molecules”, it is meant that a significant increase of the production of cytokines and/or cytolytic molecules, notably at least IFNγ and/or TNFα by activated γδ T cells, notably Vγ9Vδ2 T cells, is observed in presence of the tested antibody compared to the corresponding isotype control, said γδ T cells being co-cultured with a target cell line expressing BTN3A and PD-L1 (Daudi or SKOV3 cell lines). Typically, the activation of the production of IFNγ or TNFα by activated Vγ9Vδ2 T cells may be measured in a cellular assay by intracellular labelling with antibodies against IFNγ or TNFα assessed by flow cytometry or by an ELISA-based dosage of IFNγ or TNFα secreted by Vγ9Vδ2 T cells in the coculture medium.

As used herein, by “activating the proliferation of activated γδ T cells, notably Vγ9Vδ2 T cells”, it is meant that a significant increase of γδ T cell proliferation, notably of Vγ9Vδ2 T cells such as peripheral blood Vγ9Vδ2 T cells, is observed in presence of the tested antibody when compared to the γδ T cell proliferation, notably Vγ9Vδ2 T cells, in presence of the corresponding isotype control. Typically, Vγ9Vδ2 T cell proliferation may be measured in a cellular assay by CFSE (Carboxyfluorescein succinimidyl ester) or Cell Trace violet staining or intracellular Ki67 staining of Vγ9Vδ2 T cells from peripheral blood mononuclear cells (PBMCs), and flow cytometry analysis, or by monitoring Vγ9Vδ2 T cell compartment expansion within PBMCs with or without stimulus.

As used herein, by “activating the killing properties of γδ T cells (notably Vγ9Vδ2 T cells) against a target cell”, it is meant that a significant increase in the death of target cells is observed in killing assays wherein γδ T cells (in particular Vγ9Vδ2 T cells) are co-cultured with target cells in the presence of a bispecific antibody of the present disclosure, when compared to the death of target cells observed with the corresponding isotype control. Target cell lines are typically used in such killing assays, such target cells expressing BTN3A and PD-L1, e.g. Daudi or SKOV3 cell lines. Typically, the death of target cells may be measured by monitoring target counts and/or expression of caspase 3/7 or Annexin V at the membrane surface of target cells. In some embodiments, a bispecific antibody activates the killing of target cells expressing BTN3A and PD-L1 (such as cancer cells, e.g., ovarian cancer cell line SKOV3), as measured in a killing assay, with an EC₅₀ below 1 nM, in particular below 0.1 nM, more particularly below 0.01 nM. The killing assay may be carried out using the live imaging Incucyte® system, for instance as described in Example 5.

In all assays described herein, expression of a target protein may be native or transduced (e.g. BTN3A-expressing Raji cells with a transduced expression of PD-L1).

Bispecific antibodies of the present invention may also reduce tumor growth in vivo and/or improve overall survival, as assessed in the field in multiple pre-clinical models, as compared to monospecific antibodies targeting one or the other specificities of said bispecific antibodies, used alone or in combination. In other words, bispecific antibodies of the present invention may show superior efficacy as compared to the respective monospecific antibodies used alone or in combination.

In particular, a bispecific antibody of the present invention binding specifically to BTN3A and PD-1 may show superior efficacy, preferably at least 2, 3, 4, 5 or 10 times higher efficacy, as compared to monoclonal antibodies binding specifically to BTN3A and PD-1, respectively, when used alone or in comparison, e.g, wherein said antibodies are ICT01 and Pembrolizumab.

In particular, a bispecific antibody of the present invention binding specifically to BTN3A and PD-L1 may show superior efficacy, preferably at least 2, 3, 4, 5 or 10 times higher efficacy, as compared to monoclonal antibodies binding specifically to BTN3A and PD-L1, respectively, when used alone or in comparison, e.g, wherein said antibodies are ICT01 and Durvalumab.

Comparative assays can be carried out by any means, e.g. by in vitro killing tests against tumor cell lines, such as the ovarian cancer cell line SKOV3, or in a Mixed Lymphocyte Reaction (MLR) assay to induce an interferon-gamma production by T cells. Comparative assays can also be carried out in pre-clinical animal studies or in clinical studies, according to the standards in the field.

### Format of the Bispecific Antibody

The bispecific antibody generally combines different moieties such as full antibodies or antibody fragments which comprise the binding sites for the target antigens.

In particular, the bispecific antibody comprises a first and second antigen-binding moiety, wherein the first and/or second antigen-binding moieties may be selected from an immunoglobulin molecule and an antigen-binding fragment selected from a Fab fragment, a Fab′ fragment, a F(ab′)2 fragment, a scFab fragment, a single domain antibody or a Fv fragment such as a scFv fragment. The immunoglobulin is preferably an IgG molecule.

Various bispecific antibody formats may be generated by combining these fragments. Different criteria can be applied to classify the resulting bispecific antibodies. A main discrimination is the presence or absence of an Fc region. Bispecific antibodies with no Fc will lack Fc-mediated effector functions, such as antibody-dependent cell-mediated cytotoxicity (ADCC), antibody-dependent cellular phagocytosis (ADCP), complement fixation, and FcRn-mediated recycling, which is responsible for the long half-life of most γ immunoglobulins. Bispecific antibodies that include a Fc region can be further divided into those that exhibit a structure resembling that of an IgG molecule and those that contain additional binding sites, i.e., those with an appended or modified Ig-like structure. The different bispecific antibodies will have either a symmetric or an asymmetric architecture. For example, the majority of bispecific IgG molecules are asymmetric, while IgG fusion proteins often are symmetric in their molecular composition. A further discriminating feature is the number of binding sites. In the simplest setting, e.g., utilized in IgG molecules, a bispecific antibody contains one binding site for each antigen (1+1), i.e., is bivalent. Adding an additional binding site to one of the chains of an IgG results in tetravalent molecules with a 2+2 stoichiometry. Other formats allow to generate 1+2 or 1+3 molecules, having one binding site for one antigen and 2 or 3 binding sites for the other antigen, respectively. This can be extended by further valencies, but also by implementing further specificities, e.g., to make tri- or tetra-specific molecules. Furthermore, the number of chains needed to produce the bispecific antibody can vary. Thus, bispecific IgGs typically require four different polypeptide chains to be expressed, but a smaller number of chains, i.e., 3, 2 or only a single polypeptide chain, can be used in some formats.

Different antibody formats can be used, including the non-limiting following formats:
bispecific antibody conjugates, such as IgG₂, F(ab′)₂ or CovX-Body;
hybrid bispecific IgGs, such as hybrid IgGs and κ/λ-body common HC;
“variable domain only” bispecific antibody molecules, such as tandem scFv (taFv), triplebody, diabody (Db), dsDb, Db (kih), DART, scDb, dsFv-dsFv′, tandAbs, tripleheads, tandem dAb/VHH, triple dAb/VHH or tetravalent dAb/VHH;
CH1/CL fusion proteins, such as scFv₂-CH1/CL or VHH2-CH1/CL;
Fab fusion proteins, such as Fab-scFv (bibody), Fab-scFV₂ (tribody), Fab-Fv, Fab-dsFv, Fab-VHH or orthogonal Fab-Fab;
Fc-modified IgGs, such as IgG(kih), IgG(kih) common LC, ZW1 IgG common LC, Biclonics common LC, CrossMab (IgG-kih), scFab-IgG-(kih), Fab-scFab-IgG-(kih), orthogonal Fab IgG(kih), DuetMab, CH3 charge pairs+CH1/CL charge pairs, hinge/CH3 charge pairs, Duobody, four-in-one-CrossMab(kih), LUZ-Y common LC, LUZ-Y scFab-IgG, LUZ-Y scFab-IgG or FcFc′;
appended & Fc-modified IgGs, such as IgG(kih)-Fv, IgG(HA-TF-Fv, IgG(kih)-scFab, scFab-Fc(kih)-scFv2, scFab-Fc(kih)-scFv, half DVD-Ig, DVI-Ig (four-in-one), CrossMab-Fab;
modified Fc and CH3 fusion proteins, such as scFv-Fc (kih), scFv-Fc (CH3 charge pairs), scFV-Fc (EW-RVT), scFv-Fc (HA-TF), scFv-Fc (SEEDbody), taGv-Fc(kih), scFv-Fc(kih)-Fv, Fab-Fc(kih)-scFv, Fab-scFv-Fc(kih), Fab-scFv-Fc(BEAT), Fab-scFv-Fc (SEEDbody), DART-Fc, ScFv-CH₃(kih) or TriFabs;
appended IgGs-HC fusions, such as IgG-HC-scFv, IgG-dAb, IgG-taFv, IgG-CrossFab, IgG-orthogonal Fab, IgG(CαCβ)Fab, scFv-HC-IgG, tandem Fab-IgG (orthogonal Fab), Fab-IgG(CαCβFab), Fab-IgG(CR3) or Fab-hinge-IgG(CR3);
appended IgGs-Lc fusions, such as IgG-scFv(LC), scFv(LC)-IgG or dAb-IgG;
appended IgGs-HC&LC fusions, such as DVD-Ig, TVF-Ig, CODV-Ig, scFv1-IgG or Zybody;
Fc fusions, such as Di-diabody, scDb-Fc, taFv-Fc, scFv-Fc-scFv, HCAb-VHH, Fab-scFv-Fc, scFv₄-Ig or scFv₂-Fcab;
CH3 fusions such as Di-diabody or scDb-C_{H}3;
F(ab′)2 fusions, such as F(ab′)2-scFV₂;
CH1/CL fusion proteins, such as scFv₂-CH1-hinge/CL;
modified variable light domain, such as κ-λ chimeric

Such antibody formats and methods for their production are known from the skilled person and described, e.g. in Brinkmann U, Kontermann R E. The making of bispecific antibodies. MAbs. 2017 February/March; 9(2):182-212.

Preferred formats for the antibodies of the invention comprise an immunoglobulin G linked with another antigen-binding moiety. In such formats, the IgG may be the moiety with specificity for BTN3A or the moiety with specificity for PD-1 or PD-L1.

In particular aspects, the IgG may be linked with an antigen-binding fragment selected from a Fab fragment, a Fab′ fragment, a F(ab′)2 fragment, a scFab fragment, a single domain antibody or a Fv fragment such as a scFv fragment.

A particularly preferred format is the IgG-HC fusions or IgG-LC fusions format, in which an IgG is linked with one or more antigen-binding fragments, preferably with one or more scFvs, one or more scFabs or with one or more single domain antibodies.

Typically, preferred bispecific molecules of the present invention are IgG-scFv antibodies. Such antibodies comprise an Fc Region, a Fab fragment providing binding to one antigen and a single chain fragment (scFv) providing binding to another antigen. The Fab fragment may bind specifically to PD-1 or PD-L1 and the scFv may bind specifically to BTN3A or, preferably, the Fab fragment may bind specifically to BTN3A and the scFv may bind specifically to PD-1 or PD-L1. Each scFv unit in such constructs is made up of one variable domain from each of the heavy (VH) and light (VL) antibody chains, joined with one another via a synthetic polypeptide linker, the latter often being genetically engineered so as to be minimally immunogenic while remaining maximally resistant to proteolysis. Respective scFv units are joined by a number of techniques including incorporation of a short (usually less than 20 amino acids) polypeptide spacer bridging the two scFv units, thereby creating a bispecific single chain antibody. The design of linkers suitable for this purpose is described in the present specification and in the prior art, for example in the granted patents EP 623 679 B1, U.S. Pat. No. 5,258,498, EP 573 551 B1 and U.S. Pat. No. 5,525,491.

As provided in Figures, the bispecific molecules of the present invention may have alternative orientations in which the scFv binding Domain links either the N-terminus of heavy chain or light chain or C-terminus of heavy chain or light chain (e.g. formats IgG-HC-scFv, scFv-HC-IgG, IgG-scFv(LC) and scFv(LC)-IgG).

In some embodiments, a scFv has the structure VH-VL, wherein the C-terminal end of the VH domain is linked to the N-terminal end of the VL domain. In some embodiment, a scFv has the structure VL-VH, wherein the C-terminal end of the VL domain is linked to the N-terminal end of the VH domain.

Therefore, in particular embodiments, the bispecific antibody comprises
(i) two heavy chains having the formula [scFv-linker-IgG(H)] and two light chains having the formula [IgG(L)];
(ii) two heavy chains having the formula [IgG(H)-linker-scFv] and two light chains having the formula [IgG(L)];
(iii) two heavy chains having the formula [IgG(H)] and two light chains having the formula [scFv-linker-IgG(L)];
(iv) two heavy chains having the formula [IgG(H)] and two light chains having the formula [IgG(L)-linker-scFv];
(v) one heavy chain having the formula [scFv-linker-IgG(H)], one heavy chain having the formula [IgG(H)] and two light chains having the formula [IgG(L)];
(vi) one heavy chain having the formula [IgG(H)-linker-scFv], one heavy chain having the formula [IgG(H)] and two light chains having the formula [IgG(L)];
(vii) two heavy chains having the formula [IgG(H)], one light chain having the formula [scFv-linker-IgG(L)] and one light chain having the formula [IgG(L)];
(viii) two heavy chains having the formula [IgG(H)], one light chain having the formula [IgG(L)-linker-scFv] and one light chain having the formula [IgG(L)];
(ix) one heavy chain having the formula [IgG(H)], one heavy chain having the formula [scFv-linker-IgG Fc] and one light chain having the formula [IgG(L)]; or
(x) one heavy chain having the formula [IgG(H)-linker-scFv], one heavy chain having the formula [IgG Fc-linker-scFv] and one light chain having the formula [IgG(L)],
wherein IgG(H) and IgG(L) are, respectively, the heavy chain and light chain of the IgG molecule that specifically binds to BTN3A or PD-1 or PD-L1, in particular BTN3A, wherein IgG Fc is an IgG Fc region, and wherein scFv is the scFv fragment that specifically binds to BTN3A or PD-1 or PD-L1, in particular PD-1 or PD-L1, wherein, where more than one scFv is present, said scFvs are identical or different and, where more than one linker is present, said linkers are identical or different.

In particular embodiments, the bispecific antibody comprises two heavy chains having the formula [IgG(H)-linker-scFv] and two light chains having the formula [IgG(L)] wherein IgG(H) and IgG(L) are, respectively, the heavy chain and light chain of the IgG molecule that specifically binds to BTN3A and wherein scFv is the scFv fragment that specifically binds to PD-1 or PD-L1. Alternatively, preferred bispecific molecules of the present invention are IgG-scFab antibodies. Such antibodies comprise a Fc region, a Fab fragment providing binding to one antigen and a single chain Fab fragment (scFab) providing binding to another antigen. The Fab fragment may bind specifically to PD-1 or PD-L1 and the scFab may bind specifically to BTN3A or, preferably, the Fab fragment may bind specifically to BTN3A and the scFab may bind specifically to PD-1 or PD-L1. Each scFab unit in such constructs is made up of one Fab domain from each of the heavy (VH-CH1) and light (VL-CL) antibody chains, joined with one another via a synthetic polypeptide linker, the latter often being genetically engineered so as to be minimally immunogenic while remaining maximally resistant to proteolysis. Respective scFab units are joined by a number of techniques including incorporation of a long (usually greater than 30 amino acids) polypeptide spacer bridging the two Fab units, thereby creating a bispecific single chain antibody.

In one embodiment, the antigen-binding fragment linked to the IgG molecule is an scFab fragment.

In some embodiments of the bispecific antibody:
(i) the scFab fragment is linked to the C-terminal end of a heavy chain of the IgG molecule;
(ii) the scFab fragment is linked to the N-terminal end of a heavy chain of the IgG molecule;
(iii) the scFab fragment is linked to the C-terminal end of a light chain of the IgG molecule; or
(iv) the scFab fragment is linked to the N-terminal end of a light chain of the IgG molecule.

In some embodiments, the bispecific antibody comprises:
(i) two heavy chains having the formula [scFab-linker-IgG(H)] and two light chains having the formula [IgG(L)];
(ii) two heavy chains having the formula [IgG(H)-linker-scFab] and two light chains having the formula [IgG(L)];
(iii) two heavy chains having the formula [IgG(H)] and two light chains having the formula [IgG(L)-linker-scFab]; or
(iv) two heavy chains having the formula [IgG(H)] and two light chains having the formula [scFab-linker-IgG(L)];
wherein IgG(H) and IgG(L) are, respectively, the heavy chain and light chain of the IgG molecule that specifically binds to BTN3A or PD-1 or PD-L1, and wherein scFab is the scFab fragment that specifically binds to BTN3A or PD-1 or PD-L1, wherein, where more than one scFab is present, said scFab are identical or different and, where more than one linker is present, said linkers are identical or different.

In some embodiments, the bispecific antibody of the invention comprises at least one immunoglobulin heavy chain selected from IgG(H), IgG(H)-linker1-VH-linker2-VL, IgG(H)-linker1-VL-linker2-VH, IgG-(H), VL-linker1-VH-linker2-IgG(H), VH-linker1-VL-linker2-IgG(H), VH-CH1-linker1-VL-CL-linker2-IgG(H), IgG(H)—VH-CH1-linker1-VL-CL-linker2—, CH2-CH3-linker1-VH-linker2-VL and CH2-CH3-linker1-VH-linker2-VL, wherein IgG(H) is, the heavy chain of an IgG molecule that specifically binds to BTN3A or PD-1 or PD-L1, in particular BTN3A, wherein VH and VL are, respectively, the heavy chain variable domain and the light chain variable domain of an antibody fragment that specifically binds to BTN3A or PD-1 or PD-L1, preferably PD-1 or PD-L1, wherein CH1, CH2, CH3 are, respectively the CH1, CH2 and CH3 constant domain of the heavy chain an IgG molecule, wherein CL is the constant domain of the light chain of an IgG molecule linker 1 and linker 2 are, independently of each other, a linker, preferably a polypeptide linker. Exemplary linkers are described in the present specification and may comprise or consist of any of the sequences set forth in SEQ ID NO: 29 to 42.

The bispecific antibody of the invention may comprise two of said immunoglobulin heavy chains, wherein said two immunoglobulin heavy chains are identical or different.

In some embodiments, the bispecific antibody of the invention comprises at least one immunoglobulin light chain selected from IgG(L), IgG(L)-linker1-VH-linker2-VL, IgG(L)-linker1-VL-linker2-VH, VH-linker1-VL-linker2-IgG(L), VL-linker1-VH-linker2-IgG(L) and VH-CH1-linker1-VL-CL-linker2-IgG(L), wherein IgG(L) is the light chain of an IgG molecule that specifically binds to BTN3A or PD-1 or PD-L1, preferably BTN3A, wherein VH and VL are, respectively, the heavy chain variable domain and the light chain variable domain of an antibody fragment, e.g. scFv, that specifically binds to BTN3A or PD-1 or PD-L1, preferably PD-1 or PD-L1, wherein CH1 is the constant domain of the heavy chain of an IgG molecule, wherein CL is the constant domain of the light chain of an IgG molecule, and wherein linker 1 and linker 2 are, independently of each other, a linker, preferably a polypeptide linker. Exemplary linkers are described in the present specification and may comprise or consist of any of the sequences set forth in SEQ ID NO: 29 to 42.

The bispecific antibody of the invention may have any of the structures described in the Examples of the present specification, particularly in Table 2 and FIG. 1.

In some embodiments, a bispecific antibody binding specifically to BTN3A and PD-L1 comprises a heavy chain comprising an amino acid sequence that is at least about 95% identical to the amino acid sequence of SEQ ID NO:64 and a light chain comprising an amino acid sequence that is at least about 95% identical to the amino acid sequence of SEQ ID NO: 65.

In some embodiments, a bispecific antibody binding specifically to BTN3A and PD-L1 competes for binding to BTN3A and/or PD-L1 with a reference bispecific antibody comprising a heavy chain comprising an amino acid sequence that is at least about 95%, in particular about 100% identical to SEQ ID NO:64 and a light chain comprising an amino acid sequence that is at least about 95%, in particular about 100% identical to SEQ ID NO:65. In particular, said reference bispecific antibody is bivalent.

In some embodiments, the bispecific antibody binding specifically to BTN3A and PD-1 comprises a heavy chain comprising an amino acid sequence that is at least about 95% identical to the amino acid sequence of SEQ ID NO:62 and a light chain comprising an amino acid sequence that is at least about 95% identical to the amino acid sequence of SEQ ID NO: 63.

In some embodiments, a bispecific antibody binding specifically to BTN3A and PD-1 competes for binding to BTN3A and/or PD-1 with a reference bispecific antibody comprising a heavy chain comprising an amino acid sequence that is at least about 95%, in particular about 100% identical to SEQ ID NO:62 and a light chain comprising an amino acid sequence that is at least about 95%, in particular about 100% identical to SEQ ID NO:63. In particular, said reference bispecific antibody is bivalent.

The bispecific antibody of the invention may comprise two of said immunoglobulin heavy chains, wherein said two immunoglobulin heavy chains are identical or different.

### IgG Domain

The IgG domain of the bispecific antibody preferably comprises an intact, or full length, IgG comprising two heavy chains and two light chains, with the light chain including two domains, a variable domain (VL) and a constant domain (CL) and the heavy chain including four domains, a variable domain (VH) and three constant domains (CH1, CH2 and CH3).

The IgG as herein disclosed may be of any isotype. The choice of isotype will be typically guided by the desired effector functions, such as ADCC silencing. Exemplary isotypes are IgG1, IgG2, IgG3, and IgG4. Either of the human light chain constant regions, kappa or lambda, may be used. If desired, the class of an antibody of the present disclosure may be switched by known methods. Typically, class switching techniques may be used to convert one IgG subclass to another, for instance from IgG1 to IgG2. Thus, the effector functions of the antibodies of the present disclosure may be changed by isotype switching to, e.g., an IgG1, IgG2, IgG3, IgG4, IgD, IgA, IgE, or IgM antibody for various therapeutic uses.

Methods of generating IgG antibodies and fragments thereof are well known in the art and comprise protein expression in prokaryotic and eukaryotic cells with subsequent isolation of the antibody and usually purification to a pharmaceutically acceptable purity. For example, antibodies may be generated via any one of several methods which employ induction of in vivo production of antibody molecules, screening of immunoglobulin libraries (Orlandi et al, 1989. Proc. Natl. Acad. Sci. U.S.A. 86:3833-3837; Winter et al., 1991, Nature 349:293-299, the disclosures of which are incorporated herein by reference) or generation of monoclonal antibody molecules by cell lines in culture. For the expression of the antibodies as aforementioned in a host cell, nucleic acids encoding the respective (modified) light and heavy chains are inserted into expression vectors by standard methods. Expression is performed in appropriate eukaryotic host cells like CHO cells or HEK293 cells and the antibody is recovered from the cells (culture supernatant). General methods for recombinant production of antibodies are well-known in the state of the art and described, for example, in the review articles of Makrides, S. C., Protein Expr. Purif. 17 (1999) 183-202, Geisse, S., et al, Protein Expr. Purif. 8 (1996) 271-282, Kaufman, R. J., Mol. Biotechnol. 16 (2000) 151-160, and Werner, R. G., Drug Res. 48 (1998) 870-880.

Methods for recombinant production include, but are not limited to, the hybridoma technique, the human B-cell hybridoma technique, and the Epstein-Barr virus (EBV)-hybridoma technique (Kohler et al., 1975. Nature 256:4950497; Kozbor et al., 1985. J. Immunol. Methods 81:31-42; Cote et al., 1983. Proc. Natl. Acad. Sci. USA 80:2026-2030; Cole et al., 1984. Mol. Cell. Biol. 62:109-120, the disclosures of which are incorporated herein by reference).

Suitable methods for the production of monoclonal antibodies are also disclosed in “Monoclonal Antibodies: A manual of techniques”, H Zola (CRC Press, 1988, the disclosures of which are incorporated herein by reference) and in “Monoclonal Hybridoma Antibodies: Techniques and Applications”, J G R Hurrell (CRC Press, 1982, the disclosures of which are incorporated herein by reference).

Typically, the bispecific antibodies as herein disclosed comprises murine, camelid, humanized, human or chimeric IgGs, notably recombinant murine, humanized or human IgGs.

A human chimeric IgG antibody of the present disclosure can be produced by obtaining nucleic acid sequences encoding VL and VH domains as previously described, constructing a human chimeric antibody expression vector by inserting them into an expression vector for animal cell having genes encoding human antibody CH1-CH2-CH3 and human antibody CL, and expressing the coding sequence by introducing the expression vector into an animal cell. The CH domains of a human chimeric antibody may be any region, which belongs to human immunoglobulin, but those of IgG class are suitable and any one of subclasses belonging to IgG class, such as IgG1, IgG2, IgG3 and IgG4, can also be used. The CL of a human chimeric antibody may be any region, which belongs to Ig, and those of kappa light chain or lambda light chain can be used. Methods for producing chimeric antibodies involve conventional recombinant DNA and gene transfection techniques which are well known in the art (See Morrison S L. et al. (1984) and patent documents U.S. Pat. Nos. 5,202,238; and 5,204,244).

Methods for humanizing antibodies based on conventional recombinant DNA and gene transfection techniques are well known in the art (See, e. g., Riechmann L. et al. 1988; Neuberger M S. et al. 1985). Antibodies can be humanized using a variety of techniques known in the art including, for example, CDR-grafting (EP 239,400; PCT publication WO91/09967; U.S. Pat. Nos. 5,225,539; 5,530,101; and 5,585,089), veneering or resurfacing (EP 592,106; EP 519,596; Padlan E A (1991); Studnicka G M et al. (1994); Roguska M A. et al. (1994)), and chain shuffling (U.S. Pat. No. 5,565,332). The general recombinant DNA technology for preparation of such antibodies is also known (see European Patent Application EP 125023 and International Patent Application WO 96/02576).

Human antibodies can also be identified using various techniques known in the art, including phage display libraries (see, for example, Hoogenboom & Winter, 1991, J. Mol. Biol. 227:381; Marks er a/., 1991, J. Mol. Biol. 222:581; Cole er a/., 1985, In: Monoclonal antibodies and Cancer Therapy, Alan R. Liss, pp. 77; Boerner er a/., 1991. J. Immunol. 147:86-95, the disclosures of which are incorporated herein by reference).

Preferably, the IgG antibody as herein disclosed is a full-length antibody. Typically said IgG comprises a human Fc region, or a variant of a said region, where the region is an IgG1, IgG2, IgG3 or IgG4 region, preferably an IgG1 or IgG4 region.

Engineering the Fc region of a therapeutic monoclonal antibody or Fc fusion protein allows the generation of molecules that are better suited to the pharmacology activity required of them (Strohl, 2009, Curr Opin Biotechnol 20 (6): 685-91, the disclosures of which are incorporated herein by reference).

In some embodiments, stabilized IgG4 antibody can be used. Examples of suitable stabilized IgG4 antibodies are antibodies wherein arginine at position 409 in a heavy chain constant region of human IgG4, which is indicated in the EU index as in Kabat et al. supra, is substituted with lysine, threonine, methionine, or leucine, typically lysine (described in WO2006033386) and/or wherein the hinge region comprises a Cys-Pro-Pro-Cys sequence. Other suitable stabilized IgG4 antibodies are disclosed in WO2008145142.

In some embodiments, the IgG of the present disclosure does not comprise a Fc portion that induces antibody dependent cellular cytotoxicity (ADCC) and/or is an “Fc silent” antibody.

As used herein, the term “silent” antibody refers to an antibody that exhibits no or low ADCC activity as measured in an in vitro ADCC activity assay, in which NK cells are co-incubated with target cells in the presence of the tested antibodies during several hours before measuring NK cell activation and target cell lysis. In one embodiment, the term “no or low ADCC activity” means that the silent antibody exhibits an ADCC activity that is below 50%, for example below 10% of the ADCC activity that is observed with the corresponding wild type (non-silent) antibody for example with a wild type human IgG1 antibody. Typically, no detectable ADCC activity is observed in an in vitro ADCC activity assay with a silent antibody in comparison to a control Fab antibody. Silenced effector functions can be obtained by mutation in the Fc constant portion of the antibodies and have been described in the art: Strohl 2009 (LALA & N297A); Baudino 2008, D265A (Baudino et al., J. Immunol. 181 (2008): 6664-69, Strohl, CO Biotechnology 20 (2009): 685-91). Examples of silent IgG1 antibodies comprise mutations reducing ADCC at positions 234, 235, 239, 265, 297, 329 and/or 331 in the IgG1 Fc amino acid sequence (EU numbering). Another silent IgG1 antibody comprises the N297A mutation, which results in aglycosylated or non-glycosylated antibodies. For example, IgG1 Fc silent fragment may include any one of the following mutations or combinations of mutations: D265A/P329A; L234F/L235Q/K322Q (FQQ); L234A/L235A (LALA); L234A/L235A/K322A (LALAKA); N297Q (aglycosyl); L234F/L235E/P331S (FES); L234A/G237A; L234A/L235E; L234A/L235A/G237A/P238S/H268A/A330S/P331S; L234A/L235A/P329G (LALAPG); and G236R/L328R.

In some embodiments, the antibody of the present disclosure does not comprise an Fc domain capable of substantially binding to a FcγRIIIA (CD16) polypeptide. In some embodiments, the antibody of the present disclosure lacks all or a portion of the Fc domain (e.g. lacks a CH2 and/or CH3 domain) or comprises a Fc domain of IgG2 or IgG4 isotype. In some embodiments, one or more amino acids selected from amino acid residues can be replaced with a different amino acid residue such that the antibody has altered C2q binding and/or reduced or abolished complement dependent cytotoxicity (CDC). This approach is described in further detail in U.S. Pat. No. 6,194,551.

One approach to improve the efficacy of a therapeutic antibody is to increase its serum persistence, thereby allowing higher circulating levels, less frequent administration and reduced doses. The half-life of an IgG depends on its pH-dependent binding to the neonatal receptor FcRn. FcRn, which is expressed at the surface of endothelial cells, binds the IgG in a pH-dependent manner and protects it from degradation. Some antibodies that selectively bind the FcRn at pH 6.0, but not pH 7.4, exhibit a higher half-life in a variety of animal models. Thus, in some embodiments, the IgG may comprise in its Fc region several mutations located at the interface between the CH2 and CH3 domains, such as T250Q/M428L (Hinton et al., 2004, J Biol Chem. 279 (8): 6213-6, the disclosures of which are incorporated herein by reference) and M252Y/S254T/T256E+H433K/N434F (Vaccaro et al., 2005, Nat. Biotechnol. 23 (10): 1283-8, the disclosures of which are incorporated herein by reference), that have been shown to increase the binding affinity to FcRn and the half-life of IgG1 in vivo.

A preferred CL domain of an IgG in a bispecific antibody of the invention is a human IgG CL Kappa Domain. The amino acid sequence of an exemplary human CL Kappa Domain is:

(SEQ ID NO: 25)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

A preferred IgG1 sequence for the CH1, CH2 and CH3 Domains of the Fc Region-containing antibodies of the present invention having reduced or abolished effector function will comprise the substitutions L234F, L235E and P331S:

(SEQ ID NO: 26)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPEFEGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPASIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGX

wherein, X is a lysine (K) or is absent.

For certain antibodies whose Fc Region-containing first and second polypeptide chains are not identical, it is desirable to reduce or prevent homodimerization from occurring between the CH2-CH3 Domains of two first polypeptide chains or between the CH2-CH3 Domains of two third polypeptide chains. The CH2 and/or CH3 Domains of such polypeptide chains need not to be identical in sequence, and advantageously are modified to foster complexing between the two polypeptide chains. For example, an amino acid substitution (preferably a substitution with an amino acid comprising a bulky side group forming a “knob”, e.g., tryptophan) can be introduced into the CH2 or CH3 Domain such that steric interference will prevent interaction with a similarly mutated domain and will obligate the mutated domain to pair with a domain into which a complementary, or accommodating mutation has been engineered, i.e., “the hole” {e.g., a substitution with glycine). Such sets of mutations can be engineered into any pair of polypeptides comprising CH2-CH3 Domains that forms a Fc Region to foster heterodimerization. Methods of protein engineering to favor heterodimerization over homodimerization are well known in the art, in particular with respect to the engineering of immunoglobulin-like molecules, and are encompassed herein (see e.g., Ridgway et al. (1996), Protein Engr. 9:617-621, Atwell et al. (1997), J. Mol. Biol. 270:26-35, and Xie et al. (2005), J. Immunol. Methods 296:95-101; each of which is hereby incorporated herein by reference in its entirety).

A preferred knob is created by modifying an IgG Fc Region to contain the modification T366W. A preferred hole may also be created by modifying an IgG Fc Region to contain the modification T366S, L368A and Y407V.

A preferred IgG1 amino acid sequence for the CH2 and CH3 Domains of the first polypeptide chain of a Fc Region-containing molecule of the present invention will have the “knob-bearing” sequence:

(SEQ ID NO: 27)
APEFEGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
SIEKTISKAKGQPREPQVYTLPPCRDELTKNQVSLWCLVKGFYPSDIAVE
WESNGQPENNYKTTPPVLDSDGSFFLYSKLTVDKSRWQQGNVFSCSVMHE
ALHNHYTQKSLSLSPGX

wherein X is a lysine (K) or is absent.

A preferred IgG1 amino acid sequence for the CH2 and CH3 Domains of the second polypeptide chain of a Fc Region-containing molecule of the present invention will have the “hole-bearing” sequence:

(SEQ ID NO: 28)
APEFEGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
SIEKTISKAKGQPREPQVCTLPPSRDELTKNQVSLSCAVKGFYPSDIAVE
WESNGQPENNYKTTPPVLDSDGSFFLVSKLTVDKSRWQQGNVFSCSVMHE
ALHNHYTQKSLSLSPGX

wherein X is a lysine (K) or is absent.

As will be noted, the CH2-CH3 Domains of SEQ ID NO:27, and SEQ ID NO:28 include substitutions at positions 234, 235 and 331 with phenylalanine, glutamic acid and serine respectively, and thus form a Fc Region exhibiting decreased (or substantially no) binding to FcγRIA (CD64), FcγRIIA (CD32A), FcγRIIB (CD32B), FcγRIIIA (CD16a) or FcγRIIIB (CD16b) (relative to the binding exhibited by the wild-type Fc Region).

According to some embodiments of the invention, the first heavy chain comprises an S354C or Y349C mutation and the second heavy chain comprises a Y349C or S354C mutation.

### ScFv Domain

A bispecific antibody according to the present disclosure may comprise one scFv or more, joined to the C-terminal end of the heavy chain and/or of the light chain of the IgG domain.

In some embodiments, wherein the bispecific antibody comprises at least two scFvs, said scFvs are joined either to the C-terminal end of the light chains or to the C-terminal end of the heavy chains (as illustrated in the Figures).

In some embodiments, bispecific antibody according to the present disclosure may comprise one scFv or more, joined to the N-terminal end of the heavy chain and/or of the light chain of the IgG domain.

In some embodiments, wherein the bispecific antibody comprises two scFvs, said scFvs are joined either to the N-terminal end of the light chain or to the N-terminal end of the heavy chains (as illustrated in the Figures).

In some embodiments, the bispecific antibody may comprise four scFvs, and two may therefore be joined to the C terminal end of the light chain, while two may further be joined to the heavy chains.

In preferred embodiments, said one or more scFv(s), is linked to the C-terminal end of the heavy chain. In preferred embodiments, wherein the bispecific antibody comprises at least two scFvs, said scFvs are linked to the C-terminal end of the heavy chain.

ScFv fragments can be connected through the C terminal end or N terminal end of the heavy or light chain independently through their VH or VL domain. In specific embodiments, the scFv fragments are connected to the C terminal end of the light chain or the heavy chain of the IgG domain, via their VL domain. In specific embodiments, the scFv fragments are connected to the C terminal end of the light chain or the heavy chain of the IgG domain, via their VH domain. In specific embodiments, the scFv fragments are connected to the N terminal end of the light chain or the heavy chain of the IgG domain, via their VL domain. In specific embodiments, the scFv fragments are connected to the N terminal end of the light chain or the heavy chain of the IgG domain, via their VH domain.

Preferably, scFvs are connected to the IgG structure of the bispecific antibody as herein disclosed through a polypeptide linker. For example, a polypeptide linker can be a short peptide linker between about 5 to 40 amino acids, notably of 5 to 25 or about 10 to about 25 amino acids. The linker is usually rich in glycine for flexibility, as well as serine or threonine for solubility, but linkers comprising amino acids randomly selected from the group consisting of valine, leucine, isoleucine, serine, threonine, lysine, arginine, histidine, aspartate, glutamate, asparagine, glutamine, glycine, and proline may also be suitable. A well-suited linker according to the present disclosure contains glycine and serine residues and is for example of the format (GGGGS) p, with p is an integer comprised between 1 and 8, notably between 1 and 4, advantageously 2; 3 or 4. In some embodiments the linker L is (GGGGS) 4. In some embodiments, the linker is selected from the group consisting of the amino acid sequences SGGGGSGGGGS (SEQ ID NO: 29), SGGGGSGGGGSAP (SEQ ID NO: 30), NFSQP (SEQ ID NO: 31), KRTVA (SEQ ID NO: 32), GGGSGGGG (SEQ ID NO: 33), GGGGGGGGS (SEQ ID NO: 34), GGGGGGGGSGGGGS (SEQ ID NO: 35), THTCPPCPEPKSSDK (SEQ ID NO: 36), GGGS (SEQ ID NO: 37), EAAKEAAKGGGGS (SEQ ID NO: 38), EAAKEAAK (SEQ ID NO: 39), GGGGS (SEQ ID NO: 40), (SG)m where m=1 to 7, GGSSGSGSGSTGTSSSGTGTSAGTTGTSASTSGSGSGGGGGSGGGGSAGG (SEQ ID NO: 41) or GGGGSGGGGSGGGGSGGGGS (SEQ ID NO: 42). In some embodiments, the linker comprises an amino acid sequence selected from the sequences defined in SEQ ID NOs: 29 to 42.

It is to be noted that, where more than one scFv is present, the scFvs may be identical or different. They also may have the same or different specificities. It is however preferred that, where the bispecific antibody comprises 2 scFvs, said scFvs have the same specificity and have typically the same amino acid sequence. In embodiments, wherein the bispecific antibody comprises more than 2 and notably 4 scFv fragments, 2 of them may have a different specificity.

The VH and VL regions of the scFvs as per the present invention may be joined by a polypeptide linker sequence that can be for example: (GGGGS) 4.

ScFvs are typically murine, human or humanized scFvs, and/or are obtained from chimeric, human or humanized antibodies. Antibody fragments can be obtained using methods well known in the art (see, for example, Harlow & Lane, 1988, “Antibodies: A Laboratory Manual, Cold Spring Harbor Laboratory, New York, the disclosures of which are incorporated herein by reference). For example, antibody fragments can be prepared by proteolytic hydrolysis of the antibody or by expression in E. coli or mammalian cells (e.g. Chinese hamster ovary cell culture or other protein expression systems) of DNA encoding the fragment. Alternatively, antibody fragments can be obtained by pepsin or papain digestion of whole antibodies by conventional methods. Typically, also, the scFvs of the present disclosure can be produced by obtaining cDNA encoding the VH and VL domains, constructing DNA encoding scFv, inserting the DNA into an expression vector for prokaryote, or an expression vector for eukaryote, and then introducing the expression vector into a prokaryote or eukaryote (as appropriate) to express the scFv.

To generate a humanized scFv fragment, the well-known technology called CDR grafting may be used, which involves selecting the complementary determining regions (CDRs) from a donor scFv fragment, and grafting them onto a human scFv fragment framework of known three dimensional structure (see, e. g., WO98/45322; WO 87/02671; U.S. Pat. Nos. 5,859,205; 5,585,089; 4,816,567; EP0173494).

Also, in bispecific antibodies according to the present disclosure, scFv polypeptides may be glycosylated on one or more amino acid residues.

### ScFab Domain

A bispecific antibody according to the present disclosure may comprise one scFab or more, joined to the C-terminal end of the heavy chain and/or of the light chain of the IgG domain.

In preferred embodiments, wherein the bispecific antibody comprises two scFabs, said scFabs are joined to the C-terminal end of the light chains and/or to the C-terminal end of the heavy chains.

In some embodiments, the bispecific antibody may comprise four scFabs, and two may therefore be joined to the C terminal end of the light chain, while two may further be joined to the heavy chains.

scFab fragments can be connected through the C terminal end of the heavy or light chain independently through their VH or VL domain. In specific embodiments, the scFab fragments are connected to the C terminal end of the light chain or the heavy chain of the IgG domain, via their VL domain.

Preferably, scFabs are connected to the IgG structure of the bispecific antibody as herein disclosed through a polypeptide linker. For example, a polypeptide linker can be a short peptide linker between about 15 to 65 amino acids, notably of 25 to 65 or about 30 to about 60 amino acids. The linker is usually rich in glycine for flexibility, as well as serine or threonine for solubility, but linkers comprising randomly selected amino acids selected from the group consisting of valine, leucine, isoleucine, serine, threonine, lysine, arginine, histidine, aspartate, glutamate, asparagine, glutamine, glycine, and proline may also be suitable. In some embodiments the linker L is (GGGGS) 4. In some embodiments, the linker is selected from the group consisting of the amino acid sequences SGGGGSGGGGS (SEQ ID NO: 29), SGGGGSGGGGSAP (SEQ ID NO: 30), NFSQP (SEQ ID NO: 31), KRTVA (SEQ ID NO: 32), GGGSGGGG (SEQ ID NO: 33), GGGGSGGGGS, (SEQ ID NO: 34), GGGGSGGGGSGGGGS (SEQ ID NO: 35), THTCPPCPEPKSSDK (SEQ ID NO: 36), GGGS (SEQ ID NO: 37), EAAKEAAKGGGGS (SEQ ID NO: 38), EAAKEAAK (SEQ ID NO: 39), GGGGS (SEQ 40), ID NO: (SG)m where m=1 to 7, GGSSGSGSGSTGTSSSGTGTSAGTTGTSASTSGSGSGGGGGSGGGGSAGG (SEQ ID NO: 41) or GGGGSGGGGSGGGGSGGGGS (SEQ ID NO: 42). In some embodiments, the linker comprises an amino acid sequence selected from the sequences defined in SEQ ID NOs: 29 to 42.

It is to be noted that, where more than one scFab is present, the scFabs may be identical or different. They also may have the same or different specificities. It is however preferred that, where the bispecific antibody comprises 2 scFabs, said scFabs have the same specificity and have typically the same amino acid sequence. In embodiments, wherein the bispecific antibody comprises more than 2 and notably 4 scFab fragments, 2 of them may have a different specificity.

The VH-CH1 and VL-CL regions of the scFabs as per the present invention may be joined by a polypeptide linker sequence that can be for example:

(SEQ ID NO: 41)
GGSSGSGSGSTGTSSSGTGTSAGTTGTSASTSGSGSGGGGGSGGGGSAG
G.

ScFabs are typically murine, human or humanized scFabs, and/or are obtained from chimeric, human or humanized antibodies. Antibody fragments can be obtained using methods well known in the art (see, for example, Harlow & Lane, 1988, “Antibodies: A Laboratory Manual, Cold Spring Harbor Laboratory, New York, the disclosures of which are incorporated herein by reference). For example, antibody fragments can be prepared by proteolytic hydrolysis of the antibody or by expression in E. coli or mammalian cells (e.g. Chinese hamster ovary cell culture or other protein expression systems) of DNA encoding the fragment. Alternatively, antibody fragments can be obtained by pepsin or papain digestion of whole antibodies by conventional methods. Typically, also, the scFabs of the present disclosure can be produced by obtaining cDNA encoding the VH-CH1 and VL-CL domains, constructing DNA encoding scFab, inserting the DNA into an expression vector for prokaryote, or an expression vector for eukaryote, and then introducing the expression vector into a prokaryote or eukaryote (as appropriate) to express the scFab.

To generate a humanized scFab fragment, the well-known technology called CDR grafting may be used, which involves selecting the complementary determining regions (CDRs) from a donor scFab fragment and grafting them onto a human scFab fragment framework of known three dimensional structure (see, e. g., WO98/45322; WO 87/02671; U.S. Pat. Nos. 5,859,205; 5,585,089; 4,816,567; EP0173494).

Also, in bispecific antibodies according to the present disclosure, scFab polypeptides may be glycosylated on one or more amino acid residues.

### Bispecific Antibody Engineering

The bispecific antibodies of the present disclosure are produced by any techniques known in the art, such as, without limitation, any chemical, biological, genetic or enzymatic technique, either alone or in combination. Typically, knowing the amino acid sequence of the desired sequence, a person skilled in the art can readily produce said antibodies by standard techniques for production of polypeptides.

For instance, they can be synthesized using well-known solid phase methods, typically using a commercially available peptide synthesis apparatus (such as that made by Applied Biosystems, Foster City, California) and following the manufacturer's instructions. Alternatively, antibodies of the present disclosure can be synthesized by recombinant DNA techniques well-known in the art. For example, antibodies can be obtained as DNA expression products after incorporation of DNA sequences encoding the antibodies into expression vectors and introduction of such vectors into suitable eukaryotic or prokaryotic hosts that will express the desired antibodies, from which they can be later isolated using well-known techniques.

The bispecific antibodies of the present disclosure may comprise one or more amino acids which have been modified or derivatised. Chemical derivatives of one or more amino acids may be achieved by reaction with a functional side group. Such derivatised molecules include, for example, those molecules in which free amino groups have been derivatised to form amine hydrochlorides, p-toluene sulphonyl groups, carboxybenzoxy groups, i-butyloxycarbonyl groups, chloroacetyl groups or formyl groups. Free carboxyl groups may be derivatised to form salts, methyl and ethyl esters or other types of esters and hydrazides. Free hydroxyl groups may be derivatised to form O-acyl or O-alkyl derivatives. Also included as chemical derivatives are those peptides which contain naturally occurring amino acid derivatives of the twenty standard amino acids. For example: 4-hydroxyproline may be substituted for proline; 5-hydroxylysine may be substituted for lysine; 3-methylhistidine may be substituted for histidine; homoserine may be substituted for serine and ornithine for lysine. Derivatives also include peptides containing one or more additions or deletions as long as the requisite activity is maintained. Other included modifications are amidation, amino terminal acylation (e.g. acetylation or thioglycolic acid amidation), terminal carboxylamidation (e.g. with ammonia or methylamine), and the like terminal modifications.

Alternatively, or in addition, one or more amino acid may be glycosylated, such as N-linked glycosylation (in which glycan moieties are attached to a nitrogen of asparagine or arginine side chains) and/or O-linked glycosylation (in which glycan moieties are attached to the hydroxyl oxygen of serine, threonine, tyrosine, hydroxylysine or hydroxyproline). Methods for the production of glycosylated antibodies are well known in the art (for example, see Jefferis, 2009, Nature Reviews Drug Discovery 8:226-234, the disclosures of which are incorporated herein by reference).

Attaching highly flexible, hydrophilic molecules, such as PEG, may also increase the hydrodynamic volume of the bispecific antibody, thus improving their serum half-lives. While, the number and size of attached PEG chains can lead to partial inactivation or decreased binding affinity of the antibodies, conjugating a single PEG chain using a site-directed approach may be a good strategy. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (Cl-ClO) alkoxy- or aryloxy-polyethylene glycol or polyethylene glycol-maleimide. In some embodiments, the antibody to be pegylated is an aglycosylated antibody. Methods for pegylating proteins are known in the art and can be applied to the antibodies of the present disclosure. See for example, EP 0154 316 by Nishimura et al. and EP 0 401 384 by Ishikawa et al.

Engineered antibodies of the present disclosure further include those in which modifications have been made to framework residues within VH and/or VL, e.g. to improve the properties of the antibody. Typically, such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis. Such “backmutated” antibodies are also intended to be encompassed by the present disclosure. Another type of framework modification involves mutating one or more residues within the framework region, or even within one or more CDR regions, to remove T cell-epitopes to thereby reduce the potential immunogenicity of the antibody. This approach is also referred to as “deimmunization” and is described in further detail in U.S. Patent Publication No. 20030153043 by Carr et al.

Another modification of the antibodies that is herein contemplated is a conjugate or a protein fusion of at least the antigen-binding region of the antibody of the present disclosure to serum protein, such as human serum albumin or a fragment thereof to increase half-life of the resulting molecule.

### Anti-BTN3A Moiety

The bispecific antibodies of the invention comprise at least one moiety which binds specifically to a BTN3A receptor. In particular, said moiety has BTN3A agonist activity. Said moiety is referred to herein as the first antigen-binding moiety and can also be referred to as an anti-BTN3A moiety.

As used herein, the term “BTN3A” has its general meaning in the art. In specific embodiments, it refers to human BTN3A polypeptides including either BTN3A1 of SEQ ID NO:43, BTN3A2 of SEQ ID NO:44 or BTN3A3 of SEQ ID NO:45.

The term “BTN3A agonist” has its general meaning in the art and refers to a compound that selectively activates the BTN3A receptor. The term “BTN3A agonist” refers to any compound that can directly or indirectly stimulate the signal transduction cascade related to the BTN3A receptor. As used herein, the term “selectively activates” refers to a compound that preferentially binds to and activates BTN3A with a greater affinity and potency, respectively, than its interaction with the other sub-types or isoforms of the butyrophilin (BTN) family (BTN2). Compounds that prefer BTN3A, but that may also activate other butyrophilin sub-types, as partial or full agonists, and thus that may have multiple BTN3A activities, are contemplated.

The anti-BTN3A moiety of the invention is preferably derived from an anti-BTN3A agonist antibody. In particular, it may comprise or consist of a full anti-BTN3A immunoglobulin, or in an antigen-binding fragment binding specifically to BTN3A. Said fragment is for instance selected from a Fab fragment, a Fab′ fragment, a F(ab′)2 fragment, a single domain antibody and a Fv fragment such as a scFv fragment.

A suitable anti-BTN3A agonist antibody from which the moiety can be derived preferably exhibits at least one of the following properties:
(i) binds to BTN3A with a K_{D} of 10 nM or less, preferably with a K_{D} of 5 nM or less, still preferably 1 nM or less, as measured by SPR, for example as described in the Examples below;
(ii) cross-reacts to cynomolgus BTN3A with a K_{D} of 100 nM or less, preferably with a K_{D} of 50 nM or less, still preferably with a K_{D} of 10 nM or less, as measured by SPR, for example as described in the Examples below;
(iii) binds to human PBMCs with an EC₅₀ of 100 g/mL or below, preferably with a EC₅₀ of 50 μg/mL, still preferably of 10 μg/mL or below, as measured in a flow cytometry assay as described in the Examples below;
(iv) induces the activation of γδ T cells, typically Vγ9Vδ2 T cells, in co-culture with BTN3A expressing cells, with an EC₅₀ of 5 μg/mL or below, preferably of 2.5 μg/mL or below, still preferably of 1 μg/mL or below, as measured in a degranulation assay as described in the Examples below.

As detailed above, an anti-BTN3A agonist antibody is advantageously characterized in that it has at least one of the following properties (see WO2012/080351 and WO2020/025703 incorporated by reference for description and examples related to anti-BTN3A antibodies suitable for the present invention):
an activation of the cytolytic functions of activated γδ T cells, as measured typically by a degranulation assay (assessment of the expression of CD107 as a degranulation marker),
activation of the killing properties of γδ T cells against a target cell, as assessed typically by killing assays,
activation of the production of cytolytic molecules, as typically assessed by cytokine production measurement (e.g., IFN-γ or TNFα) by activated γδ T cells, and/or
activation of the proliferation of activated γδ T cells, as typically assessed by CFSE or Cell Trace violet staining or intracellular Ki67 staining of Vγ9Vδ2 T cells.

Examples of suitable anti-BTN3A antibodies are described in International patent application WO2020/025703, herein incorporated by reference for description and examples related to anti-BTN3A antibodies suitable for the present invention. Preferred anti-BTN3A antibodies include antibodies which comprise the VH and/or VL domain of monoclonal antibodies 7.2 and 20.1 described in WO2020/025703, or the light and heavy chains of monoclonal antibodies 7.2 and 20.1. More preferably, the anti-BTN3A antibodies comprise 1, 2 or all 3 of HCDRs1 to 3 and/or 1, 2 or all 3 of LCDRs 1 to 3 of said monoclonal antibodies 7.2 and 20.1. Said monoclonal antibodies 7.2 and 20.1 may be in a murine, humanized or chimeric form, in particular a humanized form.

Suitable anti-BTN3A antibodies according to the present disclosure also encompass structural variants of the reference antibodies, e.g. the 7.2 and 20.1 mAbs. Said structural variants according to the present disclosure exhibit functional properties that are substantially equal or superior to the corresponding functional properties of a reference molecule (e.g.: the 7.2 or 20.1 mAb). By substantially equal it is herein intended that said functional variants retain at least about 50%, 60%, 70%, 80%, 90%, 95% or 100% of the corresponding functional property of the reference molecule.

Typically, a structural variant of the reference antibody or fragment thereof, may comprise a structural variant of a VL, VH, or CDR of the reference antibody but still retains at least a substantial proportion (at least about 50%, 60%, 70%, 80%, 90%, 95% or 100%) of the affinity (typically assessed by K_{D} as measured by surface plasmon resonance (SPR), typically at 25° C., and/or the selectivity of the reference antibody or fragment thereof (e.g.: 7.2 or 20.1 mAbs). In some cases, such variant may be associated with greater affinity, selectivity and/or specificity than the parental mAb from which it derives.

Preferably, the anti-BTN3A antibody is selected from anti-BTN3A antibodies which have (a) a variable heavy chain (VH) polypeptide comprising an amino acid sequence as defined in SEQ ID NO: 1 and/or (b) a variable light chain (VL) polypeptide comprising an amino acid sequence as defined in SEQ ID NO: 2. In some embodiments, anti-BTN3A antibody comprises VH and/or VL regions having at least 80%, notably at least, 85, 90 95, 96, 97, 98, 99 or 100 percent identity with the VH and/or VL as defined in SEQ ID NO: 1 and 2 respectively.

In another aspect, said anti-BTN3A antibody comprises HCDRs1-3 of SEQ ID NO:3-5 and LCDRs1-3 of SEQ ID NO:6-8.

Other suitable anti-BTN3A antibodies as disclosed herein include those having amino acids that have been mutated by amino acid deletion, insertion or substitution, yet have at least 60, 70, 80, 90, 95, 96, 97, 98, 99 or 100 percent identity in the CDR regions with the reference CDR regions of SEQ ID NO:3-5 and SEQ ID NO:6-8. Typically, as per the present disclosure, said antibodies may have between 1, 2, 3 or 4 amino acid variations as compared to the reference CDR sequences. Variants including modifications in the CDRs as compared to the reference antibody have also been described below. It is to be noted that when variants include modifications in the CDRs, conservative substitutions described as above may be preferred.

In other embodiments, the anti-BTN3A antibody is selected from anti-BTN3A antibodies which have (a) a variable heavy chain (VH) polypeptide comprising an amino acid sequence as defined in SEQ ID NO: 48 and/or (b) a variable light chain (VL) polypeptide comprising an amino acid sequence as defined in SEQ ID NO: 49. In some embodiments, anti-BTN3A antibody comprises VH and/or VL regions having at least 80%, notably at least, 85, 90 95, 96, 97, 98, 99 or 100 percent identity with the VH and/or VL as defined in SEQ ID NO: 48 and 49 respectively.

In another aspect, said anti-BTN3A antibody comprises HCDRs1-3 of SEQ ID NO:50-52 and LCDRs1-3 of SEQ ID NO:53-55.

In another aspect, said anti-BTN3A antibody comprises HCDR1 of SEQ ID NO:50, HCDR2 of SEQ ID NO:51 or 56 to 59, HCDR3 of SEQ ID NO:52 and LCDR1 of SEQ ID NO: 53, 60 or 61, LCDR2 of SEQ ID NO: 54 and LCDR3 of SEQ ID NO:55.

Other suitable anti-BTN3A antibodies as disclosed herein include those having amino acids that have been mutated by amino acid deletion, insertion or substitution, yet have at least 60, 70, 80, 90, 95, 96, 97, 98, 99 or 100 percent identity in the CDR regions with the reference CDR regions of SEQ ID NO:50-52 and SEQ ID NO:53-55. Typically, as per the present disclosure, said antibodies may have between 1, 2, 3 or 4 amino acid variations as compared to the reference CDR sequences. Variants including modifications in the CDRs as compared to the reference antibody have also been described below. It is to be noted that when variants include modifications in the CDRs, conservative substitutions described as above may be preferred.

In still another aspect, the anti-BTN3A antibody is selected from anti-BTNA antibodies which compete for binding to BTN3A with an anti-BTN3A antibody which comprises (a) a variable heavy chain (VH) polypeptide comprising an amino acid sequence that is at least about 95%, in particular about 100% identical to SEQ ID NO: 1, and (b) a variable light chain (VL) polypeptide comprising an amino acid sequence that is at least about 95%, in particular about 100% identical to SEQ ID NO: 2, or antibodies which compete for binding to BTN3A with an anti-BTN3A antibody which comprises (a) a variable heavy chain (VH) polypeptide comprising an amino acid sequence of SEQ ID NO: 48, and (b) a variable light chain (VL) polypeptide comprising an amino acid sequence of SEQ ID NO: 49; and

In still another aspect, the anti-BTN3A antibody is selected from anti-BTN3A antibodies which compete for binding with an antibody selected from mAb 20.1 as produced by the hybridoma deposited on 24 Nov. 2010 at the Collection Nationale de Cultures de Microorganismes (CNCM), Institut Pasteur, 25 rue du Docteur Roux, 75724 Paris Cedex 15, under deposit number I-4401, and mAb 7.2 as produced by the hybridoma deposited 24 Nov. 2010 at the CNCM under deposit number I-4402.

Hybridoma has been deposited pursuant to, and in satisfaction of, the requirements of the Budapest Treaty on the International Recognition of the Deposit of Microorganisms for the Purposes of Patent Procedure (the “Budapest Treaty”) with the National Collection of Industrial, Food and Marine Bacteria (NCIMB), Ferguson Building, Craibstone Estate, Bucksburn, Aberdeen, AB21 9YA Scotland, on 17 Jan. 2023, under accession number NCIMB 44105.

A deposit of the PB21SG7-1294 seeds is maintained by Vilmorin & Cie, 4 quai de la Mégisserie, 75001 Paris, France.

In still another aspect, the anti-BTN3A antibody is as described in any of the International Patent Applications WO2012/080769; WO2012/080351, WO2020/136218, WO 2020/033923, WO 2020/033926, WO2023/161457 and in Dai et al, 2024, the content of which is herein entirely incorporated by reference.

In some embodiments, the anti-BTN3A moiety comprises an amino acid sequence that is at least about 95% identical to the amino acid sequence of SEQ ID NO:66, 67 or 72.

The first antigen-binding moiety may consist of or comprise a full anti-BTN3A antibody as defined above, or in any antigen-binding fragment thereof.

### Anti PD-1 or Anti-PD-L1 Moiety

The bispecific antibodies of the invention comprise at least one moiety which binds specifically to a molecule of the PD-1/PD-L1 axis, more particularly to PD-1 or PD-L1. Said moiety, referred to herein as a second antigen-binding moiety, can also be referred to as an anti-PD-1 moiety if it binds specifically to PD-1 or anti-PD-L1 moiety if it binds specifically to PD-L1.

The term “PD-1” has its general meaning in the art and refers to the programmed death-1 receptor also known of cluster of differentiation 279 (CD279). The term “PD-1” also refers to a type I transmembrane protein, belonging to the CD28-B7 signaling family of receptors that includes CD28, cytotoxic T-lymphocyte-associated antigen 4 (CTLA-4), inducible costimulator (ICOS), and B- and T-lymphocyte attenuator (BTLA) (Greenwald R J et al., 2005, Riley J L et aL., 2005).

The term “PD-L1” has its general meaning in the art and refers to the programmed death-ligand 1 also known as cluster of differentiation 274 (CD274) or B7 homolog 1 (B7-H1). The term “PD-L1” also refers to a type 1 transmembrane protein which, through its binding to the inhibitory checkpoint molecule PD-1 transmits an inhibitory signal based on interaction with phosphatases (SHP-1 or SHP-2) via Immunoreceptor Tyrosine-Based Switch Motif (ITSM).

The term “anti-PD-1 antibody” has its general meaning in the art and refers to an antibody with binding affinity and antagonist activity to PD-1, i.e., it inhibits the signal transduction cascade related to PD-1 and inhibits PD-1 ligand binding (PD-L1; PD-L2). Such anti-PD-1 antibody preferentially inactivates PD-1 with a greater affinity and potency, respectively, than its interaction with the other sub-types or isoforms of the CD28-B7 signaling family of receptors (CD28; CTLA-4; ICOS; BTLA). Tests and assays for determining whether a compound is a PD-1 antagonist are well known by the skilled person in the art such as described in Greenwald et al., 2005; Riley et al., 2005.

The term “anti-PD-L1 antibody” has its general meaning in the art and refers to an antibody with binding affinity to PD-L1 and with antagonist activity to PD-1, i.e. it inhibits the signal transduction cascade related to PD-1 by disrupting the interaction of PD-L1 with PD-1. Such anti-PD-L1 antibody preferentially does not inhibit the interaction of PD-L2 with PD-1. Tests and assays for determining whether a compound is a PD-1 antagonist are well known by the skilled person in the art.

Anti-PD-1 and anti-PD-L1 antibodies inhibit the binding of PD-1 and PD-L1, thereby blocking the inhibitory axis PD-1/PD-L1. Anti-PD-1 antibodies and anti-PD-L1 antibodies of the invention may also be referred to as PD-1 inhibitors and PD-L1 inhibitors, respectively.

An antibody which blocks or inhibits the PD-1/PD-L1 axis or PD-1/PD-L2 axis has its common meaning in the art and refers to an antibody which disrupts the binding between PD-1 and PD-L1 or PD-L2 and inhibits the signal transduction cascade related to PD-1. Such antibodies include anti-PD-1 antibodies and anti-PD-L1 antibodies. Inhibition of PD-1/PD-L1 axis can be assessed, for instance, by a PD-1/PD-L1 blockade assay, as described herein, e.g. by assessing the inhibition of TCR signaling in PD-L1 expressing antigen-presenting cells.

A PD-1/PD-L1 blockade assay may rely on a reporter assay based on the co-culture of PD-1 expressing effector cells which express PD-1, TCR and a reporter gene, with PD-L1 expressing antigen-presenting cells to monitor effector cell activation in presence of the different bispecific constructs. The reporter cells can be Jurkat cells, in particular Jurkat cells expressing the αβ TCR and PD-1. For instance, the reporter cells can be Jurkat-NFAT-Luci-PD-1 cells (Jurkat cells expressing the γβ TCR and PD-1 receptor with a luciferase reporter gene). The PD-L1 expressing antigen-presenting cells may be Raji cells such as Raji-PD-L1 cells. Co-culture of the two cell lines permits binding of PD-L1 to PD-1 and inhibition of TCR signaling in effector cells. Since the reporter system typically consists of an NFAT-response element (RE) upstream of the luciferase gene, the absence of TCR signaling prevents luciferase expression. In the presence of a PD-1/PD-L1 inhibitor, a bioluminescent signal is generated which can be detected and quantified. Serial dilutions of the inhibitor allow for the evaluation of potency, while assessing stability can be done by maintaining the agent at different temperatures and/or treating for different lengths of time.

Exemplary methods for assessing the properties of an anti-PD-1 antibody or an anti-PD-1 moiety are disclosed in Assessment report: Keytruda. Procedure no. EMEA/H/C/003820/0000. 21 May 2015, EMA/444458/2015 Rev 1, Committee for Medicinal Products for Human Use (CHMP), Available from: https://www.ema.europa.eu/en/documents/assessment-report/keytruda-epar-public-assessment-report_en.pdf.

Exemplary methods for assessing the properties of an anti-PD-L1 antibody or an anti-PD-L1 moiety, are disclosed in Assessment report: Imfinzi. Procedure No. EMEA/H/C/004771/0000. 26 Jul. 2018. EMA/CHMP/548232/2018, Committee for Medicinal Products for Human Use (CHMP), Available from https://www.ema.europa.eu/en/documents/assessment-report/imfinzi-epar-public-assessment-report_en.pdf.

Anti-human-PD-1 antibodies (or VH and/or VL domains derived therefrom) suitable for use in the invention can be generated using methods well known in the art. Alternatively, art recognized anti-PD-1 antibodies can be used.

Preferred anti-PD-1 antibodies useful in the generation of the bispecific antibodies of the invention include pembrolizumab (Merck; also known as KEYTRUDA®, lambrolizumab, and MK-3475; see WO2008/156712), nivolumab (also known as OPDIVO®, 5C4, BMS-936558, MDX-1106, and ONO-4538; Drugbank accession DB09035; See WO 2006/121168), dostarlimab (CAS Reg. No.: 2022215-59-2; Drugbank accession DB15627; see WO 2014/179664; also known as TSR-042 or ANB011 (Tesaro Biopharmaceutical; see WO2014/179664)), retifanlimab (CAS Reg. No.: 2079108-44-2; Drugbank accession DB15766, also known as MGA012, Macrogenics, see WO 2017/19846), EH12.2H7 (Dana Farber, abcam ab 223562), spartalizumab (also known as PDR001, Novartis; see WO 2015/112900), MEDI-0680 (AstraZeneca; also known as AMP-514; see WO 2012/145493), cemiplimab (Regeneron; also known as REGN-2810; Drugbank accession DB14707; see WO 2015/112800), pidilizumab (CT-011; see U.S. Pat. No. 7,332,582 and US2009/0123413), pucotenlimab (also known as HX008, Cas Reg. No. 2403647-03-8; Drugbank accession DB17552), zimberelimab (Drugbank accession DB17505; CAS Reg. No. 2259860-24-5; also known as GLS-010 or WBP3055 (Wuxi/Harbin Gloria Pharmaceuticals; see Si-Yang Liu et al., J. Hematol. Oncol. 70:136 (2017)), prolgolimab (CAS Reg. No. 2093956-19-3; Drugbank accession DB16740), penpulimab (also known as AK105, CAS Reg. No. 2350298-92-7; Drugbank accession DB16747), toripalimab (TAIZHOU JUNSHI PHARMA; also known as JS001, see Si-Yang Liu et al., J. Hematol. Oncol. 70:136 (2017)), tislelizumab (Beigene; also known as BGB-A317, see WO 2015/35606 and US 2015/0079109), camrelizumab (Jiangsu Hengrui Medicine; also known as SHR-1210 or INCSHR1210; see WO 2015/085847; Si-Yang Liu et al., J. Hematol. Oncol. 70:136 (2017)), AM-0001 (Armo; see WO 2017/123557), STI-1110 (Sorrento Therapeutics; see WO 2014/194302), balstilimab (Agenus; also known as AGEN2034, see WO 2017/040790), and sintilimab (Innovent; also known as IBI308, see WO 2017/024465, WO 2017/025016, WO 2017/132825, and WO 2017/133540) and SG001, which references are herein incorporated by reference.

The nucleic acid sequences and amino acid sequences of said antibodies are hereby incorporated by reference in their entirety, notably any of their VH, VL, CDRL1, CDRL2, CDRL3, CDRH1, CDRH2 and CDRH3 sequences.

In particular, the anti-PD-1 moiety in the present invention is derived from said anti-PD-1 antibodies, e.g. it comprises any of said anti-PD-1 antibodies or an antigen-binding fragment of any of said anti-PD-1 antibodies.

In particular, the anti-PD-1 moiety in the present invention may comprise the VH and/or VL domains of said antibodies, or comprises VH and/or VL with an amino acid sequence with at least 95% identity with the VH and/or VL domains of said anti-PD-1 antibodies. More preferably, the anti-PD-1 antibodies possess 1, 2 or all 3 of the CDRLs of the VL domain and/or 1, 2 or all 3 of the CDRHs of the VH domain of said anti-PD-1 antibodies.

In some embodiments, the anti-PD-1 moiety comprises an amino acid sequence that is at least about 95% identical to the amino acid sequence of SEQ ID NO:70, 71 or 73.

Other anti-PD-1 monoclonal antibodies have been described in, for example, U.S. Pat. Nos. 6,808,710, 7,488,802, 8,168,757 and 8,354,509, US Publication No. 2016/0272708, and PCT Publication Nos. WO 2009/014708, WO 03/099196, WO 2009/114335 and WO 2011/161699, WO 2006/121168, WO 2008/156712, WO 2015/112900, WO 2012/145493, WO 2015/112800, WO 2014/206107, WO 2015/35606, WO 2015/085847, WO 2014/179664, WO 2017/020291, WO 2017/020858, WO 2016/197367, WO 2017/024515, WO 2017/025051, WO 2017/123557, WO2016/106159, WO 2014/194302, WO 2017/040790, WO 2017/133540, WO 2017/132827, WO 2017/024465, WO 2017/025016, WO 2017/106061, WO 2017/19846, WO 2017/024465, WO 2017/025016, WO 2017/132825, and WO 2017/133540, each of which are herein incorporated by reference. Antibodies or antigen binding fragments thereof that compete with any of these antibodies or inhibitors for binding to PD-1 also can be used.

More preferably the anti-PD-1 antibodies comprise 1, 2 or all 3 of the CDRLs of the VL domain and/or 1, 2 or all 3 of the CDRHs of the VH domain of pembrolizumab.

Preferred anti-PD-L1 antibodies useful in the generation of the bispecific antibodies of the invention comprise durvalumab (CAS Reg. No.: 1428935-60-7, also known as MEDI 4736 and marketed as Imfinzi® by Astrazeneca), atezolizumab (CAS Reg. No.: 1380723-44-3), avelumab (CAS Reg. No.: 1537032-82-8), BMS-936559, LY3300054, pacmilimab (Proclaim-CX-072), FAZ053, envafolimab (also known as KN035), or MDX-1105.

The nucleic acid and amino acid sequences of said antibodies are hereby incorporated by reference, notably their full sequences as well as any of their VH, VL, CDRL1, CDRL2, CDRL3, CDRH1, CDRH2 and CDRH3 sequences.

In particular, the anti-PD-L1 moiety in the present invention is derived from said anti-PD-L1 antibodies, e.g. it comprises any of said anti-PD-L1 antibodies or an antigen-binding fragment of any of said anti-PD-L1 antibodies.

In particular, the anti-PD-L1 moiety in the present invention comprise the VH and/or VL domains of said anti-PD-L1 antibodies, or comprises VH and/or VL with an amino acid sequence with at least 95% identity with the VH and/or VL domains of said anti-PD-L1 antibodies. More preferably, the anti-PD-L1 antibodies possess 1, 2 or all 3 of the CDRLs of the VL Region and/or 1, 2 or all 3 of the CDRHs of the VH Domain of durvalumab, atezolizumab, avelumab, BMS-936559, LY3300054, pacmilimab, FAZ053, envafolimab, or MDX-1105.

In some embodiments, the anti-PD-L1 moiety comprises an amino acid sequence that is at least about 95% identical to the amino acid sequence of SEQ ID NO:68, 69 or 74.

More preferably the anti-PD-L1 antibodies comprise 1, 2 or all 3 of the CDRLS of the VL domain and/or 1, 2 or all 3 of the CDRHs of the VH Domain of durvalumab.

Other art recognized anti-PD-L1 antibodies which can be used include those described in, for example, U.S. Pat. Nos. 7,635,757 and 8,217,149, U.S. Publication No. 2009/0317368, and PCT Publication Nos. WO 2011/066389 and WO 2012/145493, the teachings of which also are hereby incorporated by reference. Antibodies or antigen binding fragments thereof that compete with any of these antibodies or inhibitors for binding to PD-L1 also can be used.

Suitable anti-PD-1 or PD-L1 antibodies according to the present disclosure also encompass structural variants of the reference antibodies, e.g. the anti-PD-1 and anti-PD-L1 antibodies listed above. Said structural variants may comprise sequence variations according to the present disclosure and may exhibit functional properties that are substantially equal or superior to the corresponding functional properties of a reference molecule. By substantially equal it is herein intended that said functional variants retain at least about 50%, 60%, 70%, 80%, 90%, 95% or 100% of the corresponding functional property of the reference molecule.

Typically, a structural variant of the reference antibody or fragment thereof, may comprise a structural variant of a VL, VH or CDR of the reference antibody but still retains at least a substantial proportion (at least about 50%, 60%, 70%, 80%, 90%, 95% or 100%) of the affinity typically assessed by K_{D} as measured by SPR, typically at 25° C., and/or the selectivity of the reference antibody or fragment thereof. In some cases, such variant may be associated with greater affinity, selectivity and/or specificity than the parent Ab from which it derives.

### Nucleic Acids, Vectors and Host Cells

The present disclosure also provides isolated nucleic acid molecules encoding a bispecific polypeptide as herein described, or a component polypeptide chain thereof. For example, the nucleic acid molecules may comprise any nucleotide sequence encoding the amino acid sequences provided in Table 1.

Thus, a polynucleotide of the invention may encode any polypeptide as described herein, i.e. any polypeptide chain being part of the bispecific antibody of the invention. This includes the light chains and heavy chains of the bispecific antibody, in particular of the IgG structure and/or the scFvs as previously described. The terms “nucleic acid molecule” and “polynucleotide” are used interchangeably herein and refer to a polymeric form of nucleotides of any length, either deoxyribonucleotides or ribonucleotides, or analogs thereof. By “nucleic acid molecule” we include DNA (e.g. genomic DNA or complementary DNA) and mRNA molecules, which may be single- or double-stranded. By “isolated” we mean that the nucleic acid molecule is not located or otherwise provided within a cell. Non-limiting examples of polynucleotides include a gene, a gene fragment, messenger RNA (mRNA), cDNA, recombinant polynucleotides, plasmids, vectors, isolated DNA of any sequence, isolated RNA of any sequence, nucleic acid probes, and primers.

A polynucleotide of the invention may be provided in isolated or substantially isolated form. By substantially isolated, it is meant that there may be substantial, but not total, isolation of the polypeptide from any surrounding medium. The polynucleotides may be mixed with carriers or diluents which will not interfere with their intended use and still be regarded as substantially isolated.

A nucleic acid sequence which “encodes” a selected polypeptide is a nucleic acid molecule which is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. For the purposes of the invention, such nucleic acid sequences can include, but are not limited to, cDNA from viral, prokaryotic or eukaryotic mRNA, genomic sequences from viral or prokaryotic DNA or RNA, and even synthetic DNA sequences. A transcription termination sequence may be located 3′ to the coding sequence.

Representative polynucleotides which encode examples of a heavy chain or light chain amino acid sequence of an antibody may comprise or consist of any nucleotide sequences encoding the amino acid sequences disclosed herein, for example the sequences set out in Table 1.

The present disclosure notably provides nucleic acids encoding the light chain and/or the heavy chain of the bispecific antibody. Typically, the nucleic acids may encode one, two, three or four polypeptide chains selected from the following:
(i) two heavy chains having the formula [scFv-linker-IgG(H)] and two light chains having the formula [IgG(L)];
(ii) two heavy chains having the formula [IgG(H)-linker-scFv] and two light chains having the formula [IgG(L)];
(iii) two heavy chains having the formula [IgG(H)] and two light chains having the formula [scFv-linker-IgG(L)];
(iv) two heavy chains having the formula [IgG(H)] and two light chains having the formula [IgG(L)-linker-scFv];
(v) one heavy chain having the formula [scFv-linker-IgG(H)], one heavy chain having the formula [IgG(H)] and two light chains having the formula [IgG(L)];
(vi) one heavy chain having the formula [IgG(H)-linker-scFv], one heavy chain having the formula [IgG(H)] and two light chains having the formula [IgG(L)];
(vii) two heavy chains having the formula [IgG(H)], one light chain having the formula [scFv-linker-IgG(L)] and one light chain having the formula [IgG(L)]; and
(viii) two heavy chains having the formula [IgG(H)], one light chain having the formula [IgG(L)-linker-scFv] and one light chain having the formula [IgG(L)];
(ix) one heavy chain having the formula [IgG(H)], one heavy chain having the formula [scFv-linker-IgG Fc] and one light chain having the formula [IgG(L)];
(x) one heavy chain having the formula [IgG(H)-linker-scFv], one heavy chain having the formula [IgG Fc-linker-scFv] and one light chain having the formula [IgG(L)];
(xi) two heavy chains having the formula [scFab-linker-IgG(H)] and two light chains having the formula [IgG(L)];
(xii) two heavy chains having the formula [IgG(H)-linker-scFab] and two light chains having the formula [IgG(L)];
(xiii) two heavy chains having the formula [IgG(H)] and two light chains having the formula [IgG(L)-linker-scFab]; and
(xiv) two heavy chains having the formula [IgG(H)] and two light chains having the formula [scFab-linker-IgG(L)],
wherein:
IgG(H) and IgG(L) are, respectively, the heavy chain and light chain of the IgG molecule that specifically binds to BTN3A or PD-1 or PD-L1, wherein IgG Fc is an IgG Fc region, and wherein scFv or scFab is the fragment that specifically binds to BTN3A or PD-1 or PD-L1, wherein, where more than one scFv or scFab is present, said scFvs or scFabs are identical or different and, where more than one linker is present, said linkers are identical or different.

A suitable polynucleotide sequence may alternatively be a variant of one of these specific polynucleotide sequences. For example, a variant may be a substitution, deletion or addition variant of any of the above nucleic acid sequences. A variant polynucleotide may comprise 1, 2, 3, 4, 5, up to 10, up to 20, up to 30, up to 40, up to 50, up to 75 or more nucleic acid substitutions and/or deletions from the sequences given in the sequence listing.

Suitable variants may be at least 70% homologous to a polynucleotide of any one of nucleic acid sequences disclosed herein, preferably at least 80 or 90% and more preferably at least 95%, 97% or 99% homologous thereto. Preferably homology and identity at these levels is present at least with respect to the coding regions of the polynucleotides. Methods of measuring homology are well known in the art, and it will be understood by those of skill in the art that in the present context, homology is calculated on the basis of nucleic acid identity. Such homology may exist over a region of at least 15, preferably at least 30, for instance at least 40, 60, 100, 200 or more contiguous nucleotides. Such homology may exist over the entire length of the unmodified polynucleotide sequence.

Methods of measuring polynucleotide homology or identity are known in the art. For example, the UWGCG Package provides the BESTFIT program which can be used to calculate homology (e.g. used on its default settings) (Devereux et al, 1984, Nucleic Acids Research 12:387-395; the disclosures of which are incorporated herein by reference).

The PILEUP and BLAST algorithms can also be used to calculate homology or line up sequences (typically on their default settings), for example as described in Altschul, 1993, J Mol Evol 36:290-300; Altschul ef a/, 1990, J Mol Biol 215:403-10, the disclosures of which are incorporated herein by reference).

Software for performing BLAST analysis is publicly available through the National Centre for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff & Henikoff, 1992, Proc. Natl. Acad. Sci. USA 89:10915-10919; the disclosures of which are incorporated herein by reference) alignments (B) of 50, expectation (E) of 10, M=5, N=4, and a comparison of both strands.

The BLAST algorithm performs a statistical analysis of the similarity between two sequences; see e.g. Karlin & Altschul, 1993, Proc. Natl. Acad. Sci. USA 90:5873-5787; the disclosures of which are incorporated herein by reference. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The homologue may differ from a sequence in the relevant polynucleotide by less than 3, 5, 10, 15, 20 or more mutations (each of which may be a substitution, deletion or insertion). These mutations may be measured over a region of at least 30, for instance at least 40, 60 or 100 or more contiguous nucleotides of the homologue.

In one embodiment, a variant sequence may vary from the specific sequences given in the sequence listing by virtue of the redundancy in the genetic code. A variant polynucleotide of the invention may therefore encode the same polypeptide sequence as another polynucleotide of the invention but may have a different nucleic acid sequence due to the use of different codons to encode the same amino acids.

Nucleic acid molecule can be codon-optimised for expression of the antibody polypeptide in a particular host cell, e.g. for expression in human cells (Angov, 2011, Biotechnol. J. 6(6):650-659).

A polypeptide as per the present disclosure can be produced from or delivered in the form of a polynucleotide which encodes, and is capable of, expressing it.

Polynucleotides as herein disclosed can be synthesized according to methods well known in the art, as described by way of example in Green & Sambrook (2012, Molecular Cloning—a laboratory manual, 4^{th} edition; Cold Spring Harbor Press; the disclosures of which are incorporated herein by reference).

The nucleic acid molecules of the present invention may be provided in the form of an expression cassette which includes control sequences operably linked to the inserted sequence, thus allowing for expression of the polypeptide of the invention in vivo. These expression cassettes are typically provided within vectors (e.g., plasmids or recombinant viral vectors). Such an expression cassette may be administered directly to a host subject. Alternatively, a vector comprising a polynucleotide of the invention may be administered to a host subject.

Preferably the polynucleotide is prepared and/or administered using any suitable vector, such as a plasmid, cosmid, episome, artificial chromosome, phage or a viral vector. As used herein, the terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. A suitable vector can thus be any vector which can carry enough genetic information and allowing expression of a polypeptide of the invention.

The present disclosure therefore further provides expression vectors that comprise such polynucleotide sequences. Such expression vectors are routinely constructed in the field of molecular biology and may for example involve the use of plasmid DNA and appropriate initiators, promoters, enhancers, terminator and the like such as for example polyadenylation signals which may be necessary and which are positioned in the correct orientation to allow for expression of a peptide of the invention. Examples of promoters and enhancers used in the expression vector for animal cell include early promoter and enhancer of SV40, LTR promoter and enhancer of Moloney mouse leukemia virus, promoter and enhancer of immunoglobulin H chain and the like. Any expression vector for animal cell can be used, so long as a gene encoding the human antibody C region can be inserted and expressed. Examples of suitable vectors include pAGE107, pAGE103, pHSG274, pKCR, pSG1 beta d2-4 and the like. Other examples of plasmids include replicating plasmids comprising an origin of replication, or integrative plasmids, such as for instance pUC, pcDNA, pBR, and the like. Other examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478. Other suitable vectors would be apparent to persons skilled in the art (see Green & Sambrook, supra).

The present disclosure also provides host cells that have been modified to express a polypeptide of the invention. Such cells include higher eukaryotic cell lines, such as mammalian cells or insect cells, lower eukaryotic cells, such as yeast, or prokaryotic cells such as bacterial cells. Particular examples of cells which may be modified by insertion of vectors or expression cassettes encoding for a polypeptide of the invention, include without limitation mammalian HEK293T, CHO, HeLa, NSO and COS cells. Preferably the selected cell line is not only stable, but also allows for mature glycosylation and cell surface expression of a polypeptide.

Such cell lines of the invention can be cultured using routine technique to produce a polypeptide as herein disclosed, or may be used therapeutically or prophylactically, to deliver bispecific antibodies of the present invention to a subject. Alternatively, polynucleotides, expression cassettes or vectors of the invention may be administered ex vivo to a cell obtained from a subject and the cell then reinjected to the subject.

Therefore, the present disclosure also provides:
a vector (such as an expression vector) comprising one or more nucleic acid molecules as described above; and
a host cell (such as a mammalian cell, e.g. human cell, or Chinese hamster ovary cell, e.g. CHOK1 SV cells) comprising one or more nucleic acid molecules, or a vector, as above described.

The present disclosure also relates to a method of producing a recombinant host cell expressing an antibody as herein disclosed, said method comprising the steps of: (i) introducing in vitro or ex vivo a recombinant nucleic acid or a vector as described above into a competent host cell, (ii) culturing in vitro or ex vivo the recombinant host cell obtained and (iii), optionally, selecting the cells which express and/or secrete said antibody. Such recombinant host cells can be used for the production of antibodies of the present disclosure.

The present disclosure also encompasses a method of producing a bispecific antibody as herein disclosed comprising (i) culturing a population of host cells as above described under conditions in which said polypeptide is expressed, and (ii) isolating the polypeptide therefrom. Antibodies of the present disclosure can be suitably separated from the culture medium by conventional immunoglobulin purification procedures such as, for example, protein A-Sepharose, hydroxylapatite chromatography, gel electrophoresis, dialysis, or affinity chromatography.

### Compositions

The present disclosure further encompasses compositions comprising molecules of the invention, such as the bispecific antibodies, nucleic acid molecules, vectors and cells described herein, and at least one pharmaceutically acceptable buffers, carriers and/or excipients. In a preferred embodiment, the composition comprises one or more bispecific antibody as herein disclosed.

Additional compounds may also be included in the pharmaceutical compositions, including chelating agents such as EDTA, citrate, EGTA or glutathione.

The present disclosure further encompasses a pharmaceutical composition comprising a first antigen-binding moiety that specifically binds to BTN3A and at least one second antigen-binding moiety that specifically binds to an antigen selected from PD-L1. The two antigen-binding moieties may be present as a bispecific antibody as described herein, or may be present as two independent antigen-binding moieties, e.g. one anti-BTN3A antibody and one anti-PD-L1 antibody, or antigen-binding fragment thereof.

In some embodiments, the anti-BTN3A antibody is as described in the present invention, preferably the 20.1 or 7.2 antibody, in particular ICT01 as described in the Examples. In some embodiments, the anti-PD-L1 antibody is as described in the present invention, preferably Durvalumab.

The pharmaceutical compositions can be prepared in a manner known in the art that is sufficiently storage stable and suitable for administration to humans and animals. For example, the pharmaceutical compositions may be lyophilised, e.g. through freeze drying, spray drying, spray cooling or through use of particle formation from supercritical particle formation.

By “pharmaceutically acceptable” we mean a non-toxic material that does not decrease the effectiveness of the binding activities of the bispecific antibody as herein disclosed. Pharmaceutically acceptable buffers, carriers or excipients are well-known in the art (see Remington's Pharmaceutical Sciences, 18th edition, A. R Gennaro, Ed., Mack Publishing Company (1990) and handbook of Pharmaceutical Excipients, 3rd edition, A. Kibbe, Ed., Pharmaceutical Press (2000).

The antibody polypeptides of the invention may be formulated into any type of pharmaceutical composition known in the art to be suitable for the delivery thereof.

The pharmaceutical compositions as herein disclosed may be administered via any suitable route known to those skilled in the art. Thus, possible routes of administration include parenteral (intravenous, subcutaneous and intramuscular), topical, ocular, nasal, pulmonar, buccal, oral, parenteral, vaginal and rectal. Administration from implants may also be envisioned.

In one preferred embodiment, the pharmaceutical compositions are administered parenterally, for example, intravenously, intracerebroventricularly, intraarticularly, intra-arterially, intraperitoneally, intrathecally, intraventricularly, intrasternally, intracranially, intramuscularly or subcutaneously, or they may be administered by infusion techniques. They are conveniently used in the form of a sterile aqueous solution which may contain other substances, for example, enough salts or glucose to make the solution isotonic with blood.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets.

The pharmaceutical compositions as herein disclosed are particularly suitable for parenteral, e.g. intravenous, administration.

Alternatively, the pharmaceutical compositions may be administered intranasally or by inhalation (for example, in the form of an aerosol spray presentation from a pressurised container, pump, spray or nebulizer with the use of a suitable propellant).

The pharmaceutical compositions are administered to subjects in need thereof in a pharmaceutically effective dose. A ‘therapeutically effective amount’, or ‘effective amount’, or ‘therapeutically effective’, as used herein, refers to that amount which provides a therapeutic effect for a given condition and administration regimen. This is a predetermined quantity of active material calculated to produce a desired therapeutic effect in association with the required additive and diluent, i.e. a carrier or administration vehicle. Further, it is intended to mean an amount sufficient to reduce and most preferably prevent, a clinically significant deficit in the activity, function and response of the host. Alternatively, a therapeutically effective amount is sufficient to cause an improvement in a clinically significant condition in a host. The amount of a compound may vary depending on its specific activity. Suitable dosage amounts may contain a predetermined quantity of active composition calculated to produce the desired therapeutic effect in association with the required diluent. In the methods and use for manufacture of compositions of the invention, a therapeutically effective amount of the active component is provided. A therapeutically effective amount can be determined by the ordinary skilled medical based on patient characteristics, such as age, weight, sex, condition, complications, other diseases, etc. The administration of the pharmaceutically effective dose can be carried out both by single administration in the form of an individual dose unit or else several smaller dose units and also by multiple administrations of subdivided doses at specific intervals. Alternatively, the does may be provided as a continuous infusion over a prolonged period.

Particularly preferred compositions are formulated for systemic administration.

The composition may preferably be formulated for sustained release over a period of time.

The bispecific antibody can be formulated at various concentrations, depending on the efficacy/toxicity of the polypeptide being used. For example, the formulation may comprise the active antibody polypeptide at a concentration of between 0.1 μM and 1 mM, more preferably between 1 μM and 500 μM, between 500 μM and 1 mM, between 300 μM and 700 μM, between 1 μM and 100 μM, between 100 μM and 200 μM, between 200 μM and 300 μM, between 300 μM and 400 μM, between 400 μM and 500 μM, between 500 μM and 600 μM, between 600 μM and 700 μM, between 800 μM and 900 μM or between 900 μM and 1 mM. Typically, the formulation comprises the active antibody polypeptide at a concentration of between 300 μM and 700 μM.

Typically, the therapeutic dose of the antibody polypeptide (with or without a therapeutic moiety) in a human patient will be in the range of 100 μg to 700 mg per administration (based on a body weight of 70 kg). For example, the maximum therapeutic dose may be in the range of 0.1 to 10 mg/kg per administration, e.g. between 0.1 and 5 mg/kg or between 1 and 5 mg/kg or between 0.1 and 2 mg/kg. It will be appreciated that such a dose may be administered at different intervals, as determined by the oncologist/physician; for example, a dose may be administered daily, twice-weekly, weekly, bi-weekly or monthly.

A composition as herein disclosed may be administered alone or in combination with other therapeutic agents used in the treatment of cancers such as antimetabolites, alkylating agents, anthracyclines and other cytotoxic antibiotics, vinca alkyloids, etoposide, platinum compounds, taxanes, topoisomerase inhibitors, other cytostatic drugs, antiproliferative immunosuppressants, corticosteroids, sex hormones and hormone antagonists, and other therapeutic antibodies (such as antibodies against a tumour-associated antigen or an immune checkpoint modulator). Examples of therapeutic agents that can be used in combination therapy are described in greater detail below in the section on uses of the antibodies of the disclosure.

### Uses and Methods of the Invention

The bispecific antibodies of the present disclosure have in vitro and in vivo diagnostic and therapeutic utilities. They may be used in therapy or prophylaxis. In therapeutic applications, polypeptides or compositions are administered to a subject already suffering from a disorder or condition, in an amount sufficient to cure, alleviate or partially arrest the condition or one or more of its symptoms. Such therapeutic treatment may result in a decrease in severity of disease symptoms, or an increase in frequency or duration of symptom-free periods. In some embodiments, bispecific antibodies of the present disclosure may be used in the prevention of relapse, notably in the prevention of cancer relapse. An amount adequate to accomplish this is defined as “therapeutically effective amount”. In prophylactic applications, polypeptides or compositions are administered to a subject not yet exhibiting symptoms of a disorder or condition, in an amount sufficient to prevent or delay the development of symptoms. Such an amount is defined as a “prophylactically effective amount”. The subject may have been identified as being at risk of developing the disease or condition by any suitable means. Thus, for example, these molecules can be administered to cells in culture, e.g. in vitro, ex vivo or in vivo, or in a subject, e.g., in vivo, to treat, prevent or diagnose a variety of disorders.

The bispecific antibodies of the disclosure may activate preferentially the cytolytic functions, cytokine production and/or proliferation of T cells, preferably Vγ9Vδ2 T cells, and thereby may be used in the treatment of cancer, to overcome the immunosuppressive mechanisms observed in cancer patients.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, squamous cell carcinoma of the lung, the skin or the vagina, renal cell carcinoma, prostate cancer and/or testicular tumors, non-small cell carcinoma of the lung, small cell carcinoma of the lung, endometrial carcinoma, ovarian carcinoma, endocervical adenocarcinoma, pancreatic cancer, bladder cancer, cancer of the small intestine and cancer of the esophagus and more generally any cancer that can be treated by in vivo stimulation of the activation and/or proliferation of T cells, notably γδ T cells in a subject suffering from said cancer.

Examples of cancers include, but are not limited to, hematological malignancies such as B-cell lymphoid neoplasm, T-cell lymphoid neoplasm, non-Hodgkin lymphoma (NHL), B-NHL, T-NHL, chronic lymphocytic leukemia (CLL), small lymphocytic lymphoma (SLL), mantle cell lymphoma (MCL), NK-cell lymphoid neoplasm and myeloid cell lineage neoplasm including acute myeloid leukemia.

Examples of non-hematological cancers include, but are not limited to, colon cancer, breast cancer, lung cancer, brain cancer, prostate cancer, head and neck cancer, pancreatic cancer, bladder cancer, colorectal cancer, bone cancer, cervical cancer, liver cancer, oral cancer, esophageal cancer, thyroid cancer, kidney cancer, stomach cancer, mesothelioma, ovarian cancer, testicular cancer and skin cancer.

Accordingly, the disclosure relates to a method for treating one of the disorders disclosed above, in a subject in need thereof, said method comprising a therapeutically efficient amount of a bispecific antibody as disclosed above.

The bispecific antibodies for use as disclosed above may be administered as the sole active ingredient or in conjunction with, e.g. as an adjuvant to or in combination to, other drugs e.g., cytokines, anti-viral, anti-inflammatory agents or cytotoxic, anti-proliferative, chemotherapy or anti-tumor agents, cell therapy product (e.g. γδ T cell composition) e.g., for the treatment or prevention of diseases mentioned above.

For example, the antibodies for use as disclosed above may be used in combination with cell therapy, in particular T cell therapy, notably γδ T cell therapy, chemotherapy, antineoplastic agents, or immunotherapeutic agents.

As used herein, the term “cell therapy” refers to a therapy comprising the in vivo administration of at least a therapeutically efficient amount of a cell composition to a subject in need thereof. The cells administered to the patient may be allogeneic or autologous. The term “γδ T cell therapy” refers to a cell therapy wherein the cell composition includes, as the active principle, γδ T cells, in particular Vγ9Vδ2 T cells.

A cell therapy product refers to the cell composition which is administered to said patient for therapeutic purposes. Said cell therapy product includes a therapeutically efficient dose of cells and optionally, additional excipients, adjuvants or other pharmaceutically acceptable carriers.

Suitable antineoplastic agents may include without limitation, alkylating agents (such as cyclophosphamide, mechloretamine, chlorambucil, melphalan, nitrosureas, temozolomide), anthracyclines (such as daunorubicin, doxorubicin, epirubicin, idarubicin, mitoxantrone, valrubicin), taxanes (such as Paclitaxel, Docetaxel), epothilones, inhibitors of Topoisomerase I (such as Irinotecan or Topotecan), inhibitors of Topoisomerase II (such as Etoposide, teniposide, or Tafluposide), nucleotide analogs and precursor analogs (such as azacitidine, azathioprine, capecitabine, cytarabine, fluorouracil, gemcitabine, hydroxyurea, mercaptopurine, methotrexate, or Tioguanine), peptide antibiotics (such as carboplatin, cisplatin and oxaliplatin), retinoids (such as tretinoin, alitretinoin, bexarotene), vinca alkaloids and derivatives (such as vinblastine, vincristine, vindesine, vinorelbine), targeted therapies such as kinase inhibitors (such as Ibrutinib, Idelalisib, Erlotinib, Gefitinib, Imatinib, Vemurafenib, Vismodegib), proteasome inhibitors (such as bortezomib, carfilzomib), histone deacetylase inhibitors (such as Vorinostat or Romidepsin).

Examples of immunotherapeutic agents include without limitation phosphoantigens (e.g. zoledronic acid or other bisphosphonates), anti-CTLA-4 antibodies and cytokines (such as interleukin 2 (IL-2) (Choudhry H et al, 2018, Biomed Res Int. 2018 May 6), interleukin 15 (IL-15) (Patidar M et al., Cytokine Growth Factor Rev. 2016 October; 31:49-59), interleukin 21 (IL-21) (Caccamo N. et al., PLOS One. 2012; 7 (7): e41940), or interleukin 33 (IL-33) (Duault C et al., J Immunol. 2016 Jan. 1; 196 (1): 493-502)) or their recombinant forms and their derivatives, or any cytokine capable of inducing lymphocyte activity (e.g. proliferation or cytokine production or metabolic changes). The term derivative is used for any cytokine modifications that can rely on PEGylation (e.g. conjugation to polyethylene glycol (PEG) chains), mutation such as amino acid deletion, substitution or insertion, or association with potentiating agents (for example IL15/IL 15Ra complexes fused to an IgG1 Fc, in which IL-15 is additionally mutated (asn72asp) that further increase biological activity making this complex an IL-2 and IL-15Rβγ superagonist (Rhode P R et al, Cancer Immunol Res. 2016; 4(1):49-60) (Barroso-Sousa R et al, Curr Oncol Rep. 2018 Nov. 15; 21(1):1).

The term “IL-2” has its general meaning and refers to the human interleukin-2. IL-2 is part of the body's immune response. IL-2 mainly regulates lymphocyte activity by binding to IL-2 receptor.

The term “IL-15” has its general meaning and refers to the human interleukin-15. Like IL-2, IL-15 binds to and signals through a complex composed of IL-2/IL-15 receptor beta chain and the Common gamma chain (gamma-C, CD132). IL-15 regulates the activation and proliferation of T and natural killer (NK) cells.

The term “IL-21” has its general meaning and refers to the human interleukin-21. IL-21 has been ascribed to pleiotropic properties including, but not limited to, enhancing NK cell and CD8+ T cell cytotoxicity, modulating plasma cell differentiation and inhibiting Treg cells.

The term “IL-33” has its general meaning and refers to the human interleukin-33. IL-33, considered as an alarmin released upon tissue stress or damage, is a member of the IL-1 family and binds the ST2 receptor. IL-33 is known as an effective stimulator of T_{H}1 immune cells, natural killer (NK) cells, INKT cells, and CD8 T lymphocytes.

In accordance with the foregoing, the present disclosure provides in a yet further aspect:

A method as defined above comprising co-administration, e.g., concomitantly or in sequence, of a therapeutically effective amount of a bispecific antibody of the disclosure, and at least one second drug substance, said second drug substance being an anti-proliferative agent or immunotherapeutic agents, or cytokines or a cell therapy product (such as γδ T cells), e.g. as indicated above.

Also within the scope of the present disclosure, are kits consisting of the compositions (e.g., humanized antibodies, conjugated antibodies and multispecific molecules) disclosed herein and instructions for use. The kit can further contain a least one additional reagent, or one or more additional antibodies or proteins. Kits typically include a label indicating the intended use of the contents of the kit. The term label includes any writing, or recorded material supplied on or with the kit, or which otherwise accompanies the kit. The kit may further comprise tools for diagnosing whether a patient belongs to a group that will respond to a treatment with a bispecific antibody as defined above.

In some embodiments, the bispecific antibodies of the present disclosure are capable of, and can be used in methods comprising, promoting immune activation (e.g., against tumors). In various embodiments, the present heterodimeric proteins are capable of, and can be used in methods comprising, suppressing immune inhibition (e.g., that allows tumors to survive). In various embodiments, the present heterodimeric protein provides improved immune activation and/or improved suppression of immune inhibition.

In some embodiments, the bispecific antibodies of the present disclosure are capable of, or can be used in methods comprising, modulating the amplitude of an immune response, e.g., modulating the level of effector output. In some embodiments, e.g., when used for the treatment of cancer, bispecific antibodies of the present disclosure alter the extent of immune stimulation as compared to immune inhibition to increase the amplitude of a T cell, in particular γδ T cell response, including, without limitation, stimulating increased levels of cytokine production, proliferation or target killing potential.

Another therapeutic strategy is based on the use of a humanized antibody as disclosed herein as agents, which selectively expand and/or activate T cells, in particular Vγ9Vδ2 T cells isolated from a sample of a human subject.

The disclosure thus relates to a method for treating a subject in need thereof, comprising:
(a) isolating blood cells comprising γδ T cells, notably Vγ9Vδ2 T cells, for example PBMCs from a blood sample of a subject,
(b) expanding in vitro said γδ T cells in the presence of a bispecific antibody as herein disclosed and, optionally, other tumor or accessory cells,
(c) collecting the expanded γδ T cells,
(d) optionally, formulating the expanded γδ T cells and administering a therapeutically efficient amount of said γδ T cells to the subject.

In some embodiments, the present bispecific antibodies are capable of, or find use in methods involving, causing an increase of T cells, notably γδ T cells into a tumor or the tumor microenvironment. In some embodiments, the present heterodimeric proteins are capable of, or find use in methods involving, inhibiting and/or causing a decrease in recruitment of immunosuppressive cells (e.g., myeloid-derived suppressor cells (MDSCs), regulatory T cells (Tregs), tumor associated neutrophils (TANs), M2 macrophages, and tumor associated macrophages (TAMs)) to the tumor and/or tumor microenvironment (TME). In some embodiments, the present therapies may alter the ratio of M1 versus M2 macrophages in the tumor site and/or TME to favor M1 macrophages.

In some embodiments, the herein disclosed bispecific antibodies modulate the function of γδ T cells. The disclosure further relates to the use of a bispecific antibody as herein disclosed as agents, which selectively expand Chimeric Antigen Receptor (CAR) Vγ9Vδ2 T cells. CAR γδ T cells and their use in adoptive T cell cancer immunotherapy are described for example in Mirzaei et al (Cancer Lett 2016, 380(2):413-423).

The present disclosure therefore also relates to the in vivo use of a bispecific antibody as herein disclosed as a potentiating agent of tumor cells in a T cell therapy, in particular γδ T cell therapy in a subject in need thereof, typically suffering from cancer.

As used herein, the term γδ T cell therapy refers to a therapy, which comprises the administration to a subject in need thereof of at least an efficient amount of γδ T cells. Such γδ T cells may be allogeneic or autologous. In specific embodiments, the γδ T cells can be genetically engineered by deletion or knockout or insertion or knock-in of specific genes. In specific embodiments, said γδ T cells include γδ T cells expressing chimeric antigen receptor. The γδ T cells may have been expanded and/or purified ex vivo. Alternatively, the γδ T cells may also be comprised in a cell composition comprising other blood cells, and for example other cells of the immune system. For references regarding γδ T cell therapy, please see Pauza C D. et al, Front Immunol. 2018 Jun. 8; 9:1305. doi: 10.3389, Saudemont A. et al, Front Immunol. 2018 Feb. 5; 9:153. doi: 10.3389.

Without being bound by any particular theory, a proposed mode of action of a bispecific antibody of the present disclosure is that its binding to BTN3A expressed at the surface of a tumor cell triggers a conformational change that allows its signaling to its counter-receptor on Vγ9Vδ2 T cells.

The present disclosure also relates to a method of treatment of cancer comprising administering to a subject in need thereof an efficient amount of a bispecific antibody or a composition comprising thereof, as previously disclosed.

The disclosure thus relates to a method of treatment of a subject suffering from cancer, e.g. hematological malignancies, in particular, leukemias such as acute myeloid leukemia, and having tumor cells, for example blood tumor cells, said method comprising:
administering in said subject an efficient amount of a bispecific antibody as disclosed herein, and,
administering an efficient amount of T cells, in particular γδ T cell composition in said subject,
wherein said efficient amount of bispecific antibody has the capacity to potentiate antitumor cytolysis mediated by said T cells, in particular γδ T cell composition against said tumor cells.

The present disclosure also pertains to a method for treating a subject in need thereof, said method comprising the combined (simultaneous or sequential) administration of CAR T cells, for example CAR γδ T cells, and a bispecific antibody as disclosed herein. The present disclosure also encompasses a method of contemporaneous activation and targeting of T cells, particularly γδ T cells to tumor cells comprising administering to a subject in need thereof an effective amount of a pharmaceutical composition as herein disclosed to a subject in need thereof.

The present disclosure also encompasses
a method of modulating a patient's immune response, comprising administering an effective amount of a pharmaceutical composition of the present disclosure to a subject in need thereof.
an ex vivo method for stimulating proliferation of T cells, in particular γδ T cells, comprising contacting an effective amount of a pharmaceutical composition as herein disclosed, notably a composition comprising a bispecific antibody of the present invention with a cell derived from a subject in need thereof thereby causing an ex vivo proliferation of T cells;
an in vivo method of stimulating proliferation of T cells, in particular γδ T cells, comprising administering an effective amount of a pharmaceutical composition according to the present disclosure to a subject in need thereof thereby causing an in vivo proliferation of T cells.

The present disclosure also encompasses a first antigen-binding moiety that specifically binds to BTN3A and a second antigen-binding moiety that specifically binds to an antigen selected from PD-L1 for use as a combination treatment of a patient in need thereof, preferably for treating cancer. The two antigen-binding moieties may be present as a bispecific antibody as described herein, or may be present as two independent antigen-binding moieties, e.g. one anti-BTN3A antibody and one anti-PD-L1 antibody, or antigen-binding fragment thereof.

In some embodiments, the anti-BTN3A antibody is as described in the present invention, preferably the 20.1 or 7.2 antibody, in particular ICT01 as described in the Examples. In some embodiments, the anti-PD-L1 antibody is as described in the present invention, preferably Durvalumab.

The invention will be further illustrated by the following figures and examples. However, these examples and figures should not be interpreted in any way as limiting the scope of the present disclosure.

TABLE 1
Brief description of useful amino acid and nucleotide sequences for practicing the
invention:

| SEQ | | | |
|---|---|---|---|
| ID | | Description of the | Sequence (underlined sequences are CDR |
| NO: | Type | sequence | sequences, Kabat numbering) |
| 1 | aa | Heavy chain variable | QVQLVQSGAEVKKPGASVKLSCKASGYIFTRYY |
| | | region VH of anti- | MYWVKQRPGQGLEWIGEINPNNGGTKFNEKFKN |
| | | BTN3A mAb | RATLTVDKSISTAYMELSRLRSDDTAVYYCSRED |
| | | | DYDGTPFAMDYWGQGTLVTVSS |
| 2 | aa | Light chain variable | DIQMTQSPSSLSASVGDRVTITCHASQNINVWLS |
| | | region VL of anti- | WYQQKPGKAPKLLIYKASNLHTGVPSRFTGSGS |
| | | BTN3A mAb | GTDFTFTISSLQPEDIATYYCQQGQTYPYTFGQG |
| | | | TKLEIK |
| 3 | aa | HCDR1 of anti-BTN3A | RYYMY |
| | | mAb | |
| 4 | aa | HCDR2 of anti-BTN3A | EINPNNGGTKFNEKFKN |
| | | mAb | |
| 5 | aa | HCDR3 of anti-BTN3A | EDDYDGTPFAMDY |
| | | mAb | |
| 6 | aa | LCDR1 of anti-BTN3A | HASQNINVWLS |
| | | mAb | |
| 7 | aa | LCDR2 of anti-BTN3A | KASNLHT |
| | | mAb | |
| 8 | aa | LCDR3 of anti-BTN3A | QQGQTYPYT |
| | | mAb | |
| 9 | aa | Heavy chain variable | QVQLVQSGVEVKKPGASVKVSCKASGYTFTNYY |
| | | region VH of anti-PD-1 | MYWVRQAPGQGLEWMGGINPSNGGTNFNEKFK |
| | | mAb | NRVTLTTDSSTTTAYMELKSLQFDDTAVYYCARR |
| | | | DYRFDMGFDYWGQGTTVTVSS |
| 10 | aa | Light chain variable | EIVLTQSPATLSLSPGERATLSCRASKGVSTSGY |
| | | region VL of anti-PD-1 | SYLHWYQQKPGQAPRLLIYLASYLESGVPARFSG |
| | | mAb | SGSGTDFTLTISSLEPEDFAVYYCQHSRDLPLTF |
| | | | GGGTKVEIK |
| 11 | aa | HCDR1 of anti-PD-1 | NYYMY |
| | | mAb | |
| 12 | aa | HCDR2 of anti-PD-1 | GINPSNGGTNFNEKFKN |
| | | mAb | |
| 13 | aa | HCDR3 of anti-PD-1 | RDYRFDMGFDY |
| | | mAb | |
| 14 | aa | LCDR1 or anti-PD-1 | RASKGVSTSGYSYLH |
| | | mAb | |
| 15 | aa | LCDR2 or anti-PD-1 | LASYLES |
| | | mAb | |
| 16 | aa | LCDR3 or anti-PD-1 | QHSRDLPLT |
| | | mAb | |
| 17 | aa | Heavy chain variable | EVQLVESGGGLVQPGGSLRLSCAASGFTFSRYW |
| | | region VH of anti-PD-L1 | MSWVRQAPGKGLEWVANIKQDGSEKYYVDSVK |
| | | mAb | GRFTISRDNAKNSLYLQMNSLRAEDTAVYYCARE |
| | | | GGWFGELAFDYWGQGTLVTVSS |
| 18 | aa | Light chain variable | EIVLTQSPGTLSLSPGERATLSCRASQRVSSSYL |
| | | region VL of anti-PD-L1 | AWYQQKPGQAPRLLIYDASSRATGIPDRFSGSG |
| | | mAb | SGTDFTLTISRLEPEDFAVYYCQQYGSLPWTFGQ |
| | | | GTKVEIK |
| 19 | aa | HCDR1 of anti-PD-L1 | RYWMS |
| | | mAb | |
| 20 | aa | HCDR2 of anti-PD-L1 | NIKQDGSEKYYVDSVKG |
| | | mAb | |
| 21 | aa | HCDR3 of anti-PD-L1 | EGGWFGELAFDY |
| | | mAb | |
| 22 | aa | LCDR1 of anti-PD-L1 | RASQRVSSSYLA |
| | | mAb | |
| 23 | aa | LCDR2 of anti-PD-L1 | DASSRAT |
| | | mAb | |
| 24 | aa | LCDR3 or anti-PD-L1 | QQYGSLPWT |
| | | mAb | |
| 25 | aa | CL domain | RTVAAPSVFIFPPSDEQLKSGTASVVCLLNNFYP |
| | | | REAKVQWKVDNALQSGNSQESVTEQDSKDSTY |
| | | | SLSSTLTLSKADYEKHKVYACEVTHQGLSSPVTK |
| | | | SFNRGEC |
| 26 | aa | CH1, CH2 and CH3 | ASTKGPSVFPLAPSSKSTSGGTAALGCLVKDYFP |
| | | domains | EPVTVSWNSGALTSGVHTFPAVLQSSGLYSLSSV |
| | | | VTVPSSSLGTQTYICNVNHKPSNTKVDKRVEPKS |
| | | | CDKTHTCPPCPAPEFEGGPSVFLFPPKPKDTLMI |
| | | | SRTPEVTCVVVDVSHEDPEVKFNWYVDGVEVHN |
| | | | AKTKPREEQYNSTYRVVSVLTVLHQDWLNGKEY |
| | | | KCKVSNKALPASIEKTISKAKGQPREPQVYTLPPS |
| | | | REEMTKNQVSLTCLVKGFYPSDIAVEWESNGQP |
| | | | ENNYKTTPPVLDSDGSFFLYSKLTVDKSRWQQG |
| | | | NVFSCSVMHEALHNHYTQKSLSLSPGX |
| | | | wherein X is a lysine (K) or is absent. |
| 27 | aa | Knob-bearing | APEFEGGPSVFLFPPKPKDTLMISRTPEVTCVVV |
| | | sequence for CH2 and | DVSHEDPEVKFNWYVDGVEVHNAKTKPREEQY |
| | | CH3 domains | NSTYRVVSVLTVLHQDWLNGKEYKCKVSNKALP |
| | | | ASIEKTISKAKGQPREPQVYTLPPCRDELTKNQV |
| | | | SLWCLVKGFYPSDIAVEWESNGQPENNYKTTPP |
| | | | VLDSDGSFFLYSKLTVDKSRWQQGNVFSCSVMH |
| | | | EALHNHYTQKSLSLSPGX |
| | | | wherein X is a lysine (K) or is absent. |
| 28 | aa | Hole-bearing sequence | APEFEGGPSVFLFPPKPKDTLMISRTPEVTCVVV |
| | | for CH2 and CH3 | DVSHEDPEVKFNWYVDGVEVHNAKTKPREEQY |
| | | domains | NSTYRVVSVLTVLHQDWLNGKEYKCKVSNKALP |
| | | (alternative) | ASIEKTISKAKGQPREPQVCTLPPSRDELTKNQV |
| | | | SLSCAVKGFYPSDIAVEWESNGQPENNYKTTPP |
| | | | VLDSDGSFFLVSKLTVDKSRWQQGNVFSCSVMH |
| | | | EALHNHYTQKSLSLSPGX |
| | | | wherein X is a lysine (K) or is absent. |
| 29 | aa | Linker 1 | SGGGGSGGGGS |
| 30 | aa | Linker 2 | SGGGGSGGGGSAP |
| 31 | aa | Linker 3 | NFSQP |
| 32 | aa | Linker 4 | KRTVA |
| 33 | aa | Linker 5 | GGGSGGGG |
| 34 | aa | Linker 6 | GGGGSGGGGS |
| 35 | aa | Linker 7 | GGGGSGGGGSGGGGS |
| 36 | aa | Linker 8 | THTCPPCPEPKSSDK |
| 37 | aa | Linker 9 | GGGS |
| 38 | aa | Linker 10 | EAAKEAAKGGGGS |
| 39 | aa | Linker 11 | EAAKEAAK |
| 40 | aa | Linker 12 | GGGGS |
| 41 | aa | Linker 13 | GGSSGSGSGSTGTSSSGTGTSAGTTGTSASTSG |
| | | | SGSGGGGGSGGGGSAGG |
| 42 | aa | Linker 14 | GGGGSGGGGSGGGGSGGGGS |
| 43 | aa | Human BTN3A1 | MKMASFLAFLLLNFRVCLLLLQLLMPHSAQFSVL |
| | | | GPSGPILAMVGEDADLPCHLFPTMSAETMELKW |
| | | | VSSSLRQVVNVYADGKEVEDRQSAPYRGRTSIL |
| | | | RDGITAGKAALRIHNVTASDSGKYLCYFQDGDFY |
| | | | EKALVELKVAALGSDLHVDVKGYKDGGIHLECRS |
| | | | TGWYPQPQIQWSNNKGENIPTVEAPVVADGVGL |
| | | | YAVAASVIMRGSSGEGVSCTIRSSLLGLEKTASISI |
| | | | ADPFFRSAQRWIAALAGTLPVLLLLLGGAGYFLW |
| | | | QQQEEKKTQFRKKKREQELREMAWSTMKQEQS |
| | | | TRVKLLEELRWRSIQYASRGERHSAYNEWKKAL |
| | | | FKPADVILDPKTANPILLVSEDQRSVQRAKEPQDL |
| | | | PDNPERFNWHYCVLGCESFISGRHYWEVEVGD |
| | | | RKEWHIGVCSKNVQRKGWVKMTPENGFWTMGL |
| | | | TDGNKYRTLTEPRTNLKLPKPPKKVGVFLDYETG |
| | | | DISFYNAVDGSHIHTFLDVSFSEALYPVFRILTLEP |
| | | | TALTICPA |
| 44 | aa | Human BTN3A2 | MKMASSLAFLLLNFHVSLLLVQLLTPCSAQFSVLG |
| | | | PSGPILAMVGEDADLPCHLFPTMSAETMELKWV |
| | | | SSSLRQVVNVYADGKEVEDRQSAPYRGRTSILR |
| | | | DGITAGKAALRIHNVTASDSGKYLCYFQDGDFYE |
| | | | KALVELKVAALGSNLHVEVKGYEDGGIHLECRST |
| | | | GWYPQPQIQWSNAKGENIPAVEAPVVADGVGLY |
| | | | EVAASVIMRGGSGEGVSCIIRNSLLGLEKTASISIA |
| | | | DPFFRSAQPWIAALAGTLPILLLLLAGASYFLWRQ |
| | | | QKEITALSSEIESEQEMKEMGYAATEREISLRESL |
| | | | QEELKRKKIQYLTRGEESSSDTNKSA |
| 45 | aa | Human BTN3A3 | MKMASSLAFLLLNFHVSLFLVQLLTPCSAQFSVL |
| | | | GPSGPILAMVGEDADLPCHLFPTMSAETMELRW |
| | | | VSSSLRQVVNVYADGKEVEDRQSAPYRGRTSIL |
| | | | RDGITAGKAALRIHNVTASDSGKYLCYFQDGDFY |
| | | | EKALVELKVAALGSDLHIEVKGYEDGGIHLECRST |
| | | | GWYPQPQIKWSDTKGENIPAVEAPVVADGVGLY |
| | | | AVAASVIMRGSSGGGVSCIIRNSLLGLEKTASISIA |
| | | | DPFFRSAQPWIAALAGTLPISLLLLAGASYFLWRQ |
| | | | QKEKIALSRETEREREMKEMGYAATEQEISLREK |
| | | | LQEELKWRKIQYMARGEKSLAYHEWKMALFKPA |
| | | | DVILDPDTANAILLVSEDQRSVQRAEEPRDLPDN |
| | | | PERFEWRYCVLGCENFTSGRHYWEVEVGDRKE |
| | | | WHIGVCSKNVERKKGWVKMTPENGYWTMGLTD |
| | | | GNKYRALTEPRTNLKLPEPPRKVGIFLDYETGEIS |
| | | | FYNATDGSHIYTFPHASFSEPLYPVFRILTLEPTAL |
| | | | TICPIPKEVESSPDPDLVPDHSLETPLTPGLANES |
| | | | GEPQAEVTSLLLPAHPGAEVSPSATTNQNHKLQA |
| | | | RTEALY |
| 46 | DNA | Heavy chain variable | CAGGTCCAACTGGTGCAGTCTGGGGCTGAAGT |
| | | region VH of anti- | GAAGAAGCCTGGGGCTTCAGTGAAGTTGTCCT |
| | | BTN3A mAb | GCAAGGCTTCTGGCTACATCTTCACCAGATACT |
| | | | ATATGTATTGGGTGAAGCAGAGGCCTGGACAA |
| | | | GGCCTTGAGTGGATTGGAGAGATTAATCCTAA |
| | | | CAATGGTGGTACTAAGTTCAATGAGAAGTTCAA |
| | | | GAACAGGGCCACACTGACTGTAGACAAATCCA |
| | | | TCAGCACAGCATACATGGAGCTCAGCAGGCTG |
| | | | AGATCTGACGACACGGCGGTCTATTATTGTTCA |
| | | | AGAGAGGATGATTACGACGGGACCCCCTTTGC |
| | | | TATGGACTACTGGGGTCAAGGAACCCTGGTCA |
| | | | CCGTCTCCTCAGCCTCCACCAAGGGCCCATCG |
| | | | GTCTTCCCCCTGGCACCCTCCTCCAAGAGCAC |
| | | | CTCTGGGGGCACAGCGGCCCTGGGCTGCCTG |
| | | | GTCAAGGACTACTTCCCCGAACCGGTGACGGT |
| | | | GTCGTGGAACTCAGGCGCCCTGACCAGCGGC |
| | | | GTGCACACCTTCCCGGCTGTCCTACAGTCCTC |
| | | | AGGACTCTACTCCCTCAGCAGCGTGGTGACCG |
| | | | TGCCCTCCAGCAGCTTGGGCACCCAGACCTAC |
| | | | ATCTGCAACGTGAATCACAAGCCCAGCAACAC |
| | | | CAAGGTGGACAAGAGAGTTGAGCCCAAATCTT |
| | | | GTGACAAAACTCACACATGCCCACCGTGCCCA |
| | | | GCACCTGAATTCGAGGGGGGACCGTCAGTCTT |
| | | | CCTCTTCCCCCCAAAACCCAAGGACACCCTCA |
| | | | TGATCTCCCGGACCCCTGAGGTCACATGCGTG |
| | | | GTGGTGGACGTGAGCCACGAAGACCCTGAGG |
| | | | TCAAGTTCAACTGGTACGTGGACGGCGTGGAG |
| | | | GTGCATAATGCCAAGACAAAGCCGCGGGAGGA |
| | | | GCAGTACAACAGCACGTACCGTGTGGTCAGCG |
| | | | TCCTCACCGTCCTGCACCAGGACTGGCTGAAT |
| | | | GGCAAGGAGTACAAGTGCAAGGTCTCCAACAA |
| | | | AGCCCTCCCAGCCTCCATCGAGAAAACCATCT |
| | | | CCAAAGCCAAAGGGCAGCCCCGAGAACCACA |
| | | | GGTGTACACCCTGCCCCCATCCCGGGAAGAGA |
| | | | TGACCAAGAACCAGGTCAGCCTGACCTGCCTG |
| | | | GTCAAAGGCTTCTATCCCAGCGACATCGCCGT |
| | | | GGAGTGGGAGAGCAATGGGCAGCCGGAGAAC |
| | | | AACTACAAGACCACGCCTCCCGTGCTGGACTC |
| | | | CGACGGCTCCTTCTTCCTCTATAGCAAGCTCAC |
| | | | CGTGGACAAGAGCAGGTGGCAGCAGGGGAAC |
| | | | GTCTTCTCATGCTCCGTGATGCATGAGGCTCT |
| | | | GCACAACCACTACACGCAGAAGAGCCTCTCCC |
| | | | TGTCTCCGGGTTGA |
| 47 | DNA | Light chain variable | GACATCCAGATGACCCAGTCTCCATCCAGTCT |
| | | region VL of anti- | GTCTGCATCCGTAGGAGACAGAGTCACCATCA |
| | | BTN3A mAb | CTTGCCATGCCAGTCAGAACATTAATGTTTGGT |
| | | | TATCTTGGTACCAGCAGAAACCAGGAAAAGCC |
| | | | CCTAAACTCTTGATCTATAAGGCTTCCAACTTG |
| | | | CACACAGGCGTCCCATCAAGATTTACTGGCAG |
| | | | TGGATCTGGAACAGATTTCACATTCACCATCAG |
| | | | CAGCCTGCAGCCTGAAGACATTGCCACTTACT |
| | | | ACTGTCAACAGGGTCAAACTTATCCATACACGT |
| | | | TCGGACAGGGGACCAAGCTGGAGATCAAACGA |
| | | | ACTGTGGCTGCACCATCTGTCTTCATCTTCCCG |
| | | | CCATCTGATGAGCAGTTGAAATCTGGAACTGC |
| | | | CTCTGTTGTGTGCCTGCTGAATAACTTCTATCC |
| | | | CAGAGAGGCCAAAGTACAGTGGAAGGTGGATA |
| | | | ACGCCCTCCAATCGGGTAACTCCCAGGAGAGT |
| | | | GTCACAGAGCAGGACAGCAAGGACAGCACCTA |
| | | | CAGCCTCAGCAGCACCCTGACGCTGAGCAAAG |
| | | | CAGACTACGAGAAACACAAAGTCTACGCCTGC |
| | | | GAAGTCACCCATCAGGGCCTGAGCTCGCCCGT |
| | | | CACAAAGAGCTTCAACAGGGGAGAGTGTTAG |
| 48 | aa | Heavy chain variable | QVQLVQSGAEVKKPGASVKVSCKASGYTFTRYY |
| | | region VH of anti- | LYWVKQRPGQGLEWIGEINPNNGGTKFNEKFKS |
| | | BTN3A mAb 20.1 | RATMTVDKSTRTTYMELSSLRSEDTAVYYCSRE |
| | | (humanized) | DDYDGTPDAMDYWGQGTLVTVSS |
| 49 | aa | Light chain variable | DIQMTQSPSSLSASVGDRVTITCHASQNINLWLS |
| | | region VL of anti- | WYQQKPGKAPKLLIYRASNLHTGVPSRFSGSGS |
| | | BTN3A mAb 20.1 | ATDFTFTISSLQPEDIATYYCQQGHSYPYTFGGG |
| | | (humanized) | TKVDIK |
| 50 | aa | HCDR1 of anti-BTN3A | RYYLY |
| | | mAb 20.1 | |
| 51 | aa | HCDR2 of anti-BTN3A | EINPNNGGTKFNEKFKS |
| | | mAb 20.1 | |
| 52 | aa | HCDR3 of anti-BTN3A | EDDYDGTPDAMDY |
| | | mAb 20.1 | |
| 53 | aa | LCDR1 of anti-BTN3A | HASQNINLWLS |
| | | mAb 20.1 | |
| 54 | aa | LCDR2 of anti-BTN3A | RASNLHT |
| | | mAb 20.1 | |
| 55 | aa | LCDR3 of anti-BTN3A | QQGHSYPYT |
| | | mAb 20.1 | |
| 56 | aa | Variant HCDR2 anti- | EINPSNGGTNFNEKFKS |
| | | BTN3A mAb 20.1 | |
| 57 | aa | Variant HCDR2 anti- | EINPSNGGTNFAQKFQG |
| | | BTN3A mAb 20.1 | |
| 58 | aa | Variant HCDR2 anti- | EINPSNGGTNFNEKLKS |
| | | BTN3A mAb 20.1 | |
| 59 | aa | Variant HCDR2 anti- | EINPSNGGTNFNESLKS |
| | | BTN3A mAb 20.1 | |
| 60 | aa | Variant LCDR1 anti- | HASQNINVWLS |
| | | BTN3A mAb 20.1 | |
| 61 | aa | Variant LCDR1 anti- | RASQGISVWLS |
| | | BTN3A mAb 20.1 | |
| 62 | aa | heavy chain of bsAb001 | QVQLVQSGAEVKKPGASVKLSCKASGYIFTRYY |
| | | | MYWVKQRPGQGLEWIGEINPNNGGTKFNEKFKN |
| | | | RATLTVDKSISTAYMELSRLRSDDTAVYYCSRED |
| | | | DYDGTPFAMDYWGQGTLVTVSSASTKGPSVFPL |
| | | | APSSKSTSGGTAALGCLVKDYFPEPVTVSWNSG |
| | | | ALTSGVHTFPAVLQSSGLYSLSSVVTVPSSSLGT |
| | | | QTYICNVNHKPSNTKVDKRVEPKSCDKTHTCPPC |
| | | | PAPEFEGGPSVFLFPPKPKDTLMISRTPEVTCVV |
| | | | VDVSHEDPEVKFNWYVDGVEVHNAKTKPREEQY |
| | | | NSTYRVVSVLTVLHQDWLNGKEYKCKVSNKALP |
| | | | ASIEKTISKAKGQPREPQVYTLPPSREEMTKNQV |
| | | | SLTCLVKGFYPSDIAVEWESNGQPENNYKTTPPV |
| | | | LDSDGSFFLYSKLTVDKSRWQQGNVFSCSVMHE |
| | | | ALHNHYTQKSLSLSPGGGGGSGGGGSGGGGSG |
| | | | GGGSQVQLVQSGVEVKKPGASVKVSCKASGYTF |
| | | | TNYYMYWVRQAPGQGLEWMGGINPSNGGTNFN |
| | | | EKFKNRVTLTTDSSTTTAYMELKSLQFDDTAVYY |
| | | | CARRDYRFDMGFDYWGQGTTVTVSSGGGGSG |
| | | | GGGSGGGGSGGGGSEIVLTQSPATLSLSPGERA |
| | | | TLSCRASKGVSTSGYSYLHWYQQKPGQAPRLLI |
| | | | YLASYLESGVPARFSGSGSGTDFTLTISSLEPEDF |
| | | | AVYYCQHSRDLPLTFGGGTKVEIK |
| 63 | aa | light chain of bsAb001 | DIQMTQSPSSLSASVGDRVTITCHASQNINVWLS |
| | | | WYQQKPGKAPKLLIYKASNLHTGVPSRFTGSGS |
| | | | GTDFTFTISSLQPEDIATYYCQQGQTYPYTFGQG |
| | | | TKLEIKRTVAAPSVFIFPPSDEQLKSGTASVVCLL |
| | | | NNFYPREAKVQWKVDNALQSGNSQESVTEQDS |
| | | | KDSTYSLSSTLTLSKADYEKHKVYACEVTHQGLS |
| | | | SPVTKSFNRGEC |
| 64 | aa | heavy chain of bsAb003 | QVQLVQSGAEVKKPGASVKLSCKASGYIFTRYY |
| | | | MYWVKQRPGQGLEWIGEINPNNGGTKFNEKFKN |
| | | | RATLTVDKSISTAYMELSRLRSDDTAVYYCSRED |
| | | | DYDGTPFAMDYWGQGTLVTVSSASTKGPSVFPL |
| | | | APSSKSTSGGTAALGCLVKDYFPEPVTVSWNSG |
| | | | ALTSGVHTFPAVLQSSGLYSLSSVVTVPSSSLGT |
| | | | QTYICNVNHKPSNTKVDKRVEPKSCDKTHTCPPC |
| | | | PAPEFEGGPSVFLFPPKPKDTLMISRTPEVTCVV |
| | | | VDVSHEDPEVKFNWYVDGVEVHNAKTKPREEQY |
| | | | NSTYRVVSVLTVLHQDWLNGKEYKCKVSNKALP |
| | | | ASIEKTISKAKGQPREPQVYTLPPSREEMTKNQV |
| | | | SLTCLVKGFYPSDIAVEWESNGQPENNYKTTPPV |
| | | | LDSDGSFFLYSKLTVDKSRWQQGNVFSCSVMHE |
| | | | ALHNHYTQKSLSLSPGGGGGSGGGGSGGGGSG |
| | | | GGGSEVQLVESGGGLVQPGGSLRLSCAASGFTF |
| | | | SRYWMSWVRQAPGKGLEWVANIKQDGSEKYYV |
| | | | DSVKGRFTISRDNAKNSLYLQMNSLRAEDTAVYY |
| | | | CAREGGWFGELAFDYWGQGTLVTVSSGGGGSG |
| | | | GGGSGGGGSGGGGSEIVLTQSPGTLSLSPGERA |
| | | | TLSCRASQRVSSSYLAWYQQKPGQAPRLLIYDA |
| | | | SSRATGIPDRFSGSGSGTDFTLTISRLEPEDFAVY |
| | | | YCQQYGSLPWTFGQGTKVEIK |
| 65 | aa | light chain of bsAb003 | DIQMTQSPSSLSASVGDRVTITCHASQNINVWLS |
| | | | WYQQKPGKAPKLLIYKASNLHTGVPSRFTGSGS |
| | | | GTDFTFTISSLQPEDIATYYCQQGQTYPYTFGQG |
| | | | TKLEIKRTVAAPSVFIFPPSDEQLKSGTASVVCLL |
| | | | NNFYPREAKVQWKVDNALQSGNSQESVTEQDS |
| | | | KDSTYSLSSTLTLSKADYEKHKVYACEVTHQGLS |
| | | | SPVTKSFNRGEC |
| 66 | aa | scFv VH-VL anti- | QVQLVQSGAEVKKPGASVKLSCKASGYIFTRYY |
| | | BTN3A | MYWVKQRPGQGLEWIGEINPNNGGTKFNEKFKN |
| | | | RATLTVDKSISTAYMELSRLRSDDTAVYYCSRED |
| | | | DYDGTPFAMDYWGQGTLVTVSSGGGGSGGGG |
| | | | SGGGGSGGGGSDIQMTQSPSSLSASVGDRVTIT |
| | | | CHASQNINVWLSWYQQKPGKAPKLLIYKASNLHT |
| | | | GVPSRFTGSGSGTDFTFTISSLQPEDIATYYCQQ |
| | | | GQTYPYTFGQGTKLEIK |
| 67 | aa | scFv VL-VH anti- | DIQMTQSPSSLSASVGDRVTITCHASQNINVWLS |
| | | BTN3A | WYQQKPGKAPKLLIYKASNLHTGVPSRFTGSGS |
| | | | GTDFTFTISSLQPEDIATYYCQQGQTYPYTFGQG |
| | | | TKLEIKGGGGSGGGGSGGGGSGGGGSQVQLVQ |
| | | | SGAEVKKPGASVKLSCKASGYIFTRYYMYWVKQ |
| | | | RPGQGLEWIGEINPNNGGTKFNEKFKNRATLTVD |
| | | | KSISTAYMELSRLRSDDTAVYYCSREDDYDGTPF |
| | | | AMDYWGQGTLVTVSS |
| 68 | aa | scFv VH-VL anti-PD-L1 | EVQLVESGGGLVQPGGSLRLSCAASGFTFSRYW |
| | | | MSWVRQAPGKGLEWVANIKQDGSEKYYVDSVK |
| | | | GRFTISRDNAKNSLYLQMNSLRAEDTAVYYCARE |
| | | | GGWFGELAFDYWGQGTLVTVSSGGGGSGGGG |
| | | | SGGGGSGGGGSEIVLTQSPGTLSLSPGERATLS |
| | | | CRASQRVSSSYLAWYQQKPGQAPRLLIYDASSR |
| | | | ATGIPDRFSGSGSGTDFTLTISRLEPEDFAVYYCQ |
| | | | QYGSLPWTFGQGTKVEIK |
| 69 | aa | scFv VL-VH anti-PD-L1 | EIVLTQSPGTLSLSPGERATLSCRASQRVSSSYL |
| | | | AWYQQKPGQAPRLLIYDASSRATGIPDRFSGSG |
| | | | SGTDFTLTISRLEPEDFAVYYCQQYGSLPWTFGQ |
| | | | GTKVEIKGGGGSGGGGSGGGGSGGGGSEVQLV |
| | | | ESGGGLVQPGGSLRLSCAASGFTFSRYWMSWV |
| | | | RQAPGKGLEWVANIKQDGSEKYYVDSVKGRFTI |
| | | | SRDNAKNSLYLQMNSLRAEDTAVYYCAREGGWF |
| | | | GELAFDYWGQGTLVTVSS |
| 70 | aa | scFv VH-VL anti-PD-1 | QVQLVQSGVEVKKPGASVKVSCKASGYTFTNYY |
| | | | MYWVRQAPGQGLEWMGGINPSNGGTNFNEKFK |
| | | | NRVTLTTDSSTTTAYMELKSLQFDDTAVYYCARR |
| | | | DYRFDMGFDYWGQGTTVTVSSGGGGSGGGGS |
| | | | GGGGSGGGGSEIVLTQSPATLSLSPGERATLSC |
| | | | RASKGVSTSGYSYLHWYQQKPGQAPRLLIYLAS |
| | | | YLESGVPARFSGSGSGTDFTLTISSLEPEDFAVY |
| | | | YCQHSRDLPLTFGGGTKVEIK |
| 71 | aa | scFv VL-VH anti-PD-1 | EIVLTQSPATLSLSPGERATLSCRASKGVSTSGY |
| | | | SYLHWYQQKPGQAPRLLIYLASYLESGVPARFSG |
| | | | SGSGTDFTLTISSLEPEDFAVYYCQHSRDLPLTF |
| | | | GGGTKVEIKGGGGSGGGGSGGGGSGGGGSQV |
| | | | QLVQSGVEVKKPGASVKVSCKASGYTFTNYYMY |
| | | | WVRQAPGQGLEWMGGINPSNGGTNFNEKFKNR |
| | | | VTLTTDSSTTTAYMELKSLQFDDTAVYYCARRDY |
| | | | RFDMGFDYWGQGTTVTVSS |
| 72 | aa | scFab anti-BTN3A | QVQLVQSGAEVKKPGASVKLSCKASGYIFTRYY |
| | | | MYWVKQRPGQGLEWIGEINPNNGGTKFNEKFKN |
| | | | RATLTVDKSISTAYMELSRLRSDDTAVYYCSRED |
| | | | DYDGTPFAMDYWGQGTLVTVSSASTKGPSVFPL |
| | | | APSSKSTSGGTAALGCLVKDYFPEPVTVSWNSG |
| | | | ALTSGVHTFPAVLQSSGLYSLSSVVTVPSSSLGT |
| | | | QTYICNVNHKPSNTKVDKKVEPKSCDKTHTGGS |
| | | | SGSGSGSTGTSSSGTGTSAGTTGTSASTSGSGS |
| | | | GGGGGSGGGGSAGGDIQMTQSPSSLSASVGDR |
| | | | VTITCHASQNINVWLSWYQQKPGKAPKLLIYKAS |
| | | | NLHTGVPSRFTGSGSGTDFTFTISSLQPEDIATYY |
| | | | CQQGQTYPYTFGQGTKLEIKRTVAAPSVFIFPPS |
| | | | DEQLKSGTASVVCLLNNFYPREAKVQWKVDNAL |
| | | | QSGNSQESVTEQDSKDSTYSLSSTLTLSKADYEK |
| | | | HKVYACEVTHQGLSSPVTKSFNRGEC |
| 73 | aa | scFab anti-PD-1 | QVQLVQSGVEVKKPGASVKVSCKASGYTFTNYY |
| | | | MYWVRQAPGQGLEWMGGINPSNGGTNFNEKFK |
| | | | NRVTLTTDSSTTTAYMELKSLQFDDTAVYYCARR |
| | | | DYRFDMGFDYWGQGTTVTVSSASTKGPSVFPLA |
| | | | PSSKSTSGGTAALGCLVKDYFPEPVTVSWNSGA |
| | | | LTSGVHTFPAVLQSSGLYSLSSVVTVPSSSLGTQ |
| | | | TYICNVNHKPSNTKVDKKVEPKSCDKTHTGGSS |
| | | | GSGSGSTGTSSSGTGTSAGTTGTSASTSGSGSG |
| | | | GGGGSGGGGSAGGEIVLTQSPATLSLSPGERAT |
| | | | LSCRASKGVSTSGYSYLHWYQQKPGQAPRLLIYL |
| | | | ASYLESGVPARFSGSGSGTDFTLTISSLEPEDFA |
| | | | VYYCQHSRDLPLTFGGGTKVEIKRTVAAPSVFIFP |
| | | | PSDEQLKSGTASVVCLLNNFYPREAKVQWKVDN |
| | | | ALQSGNSQESVTEQDSKDSTYSLSSTLTLSKADY |
| | | | EKHKVYACEVTHQGLSSPVTKSFNRGEC |
| 74 | aa | scFab anti-PD-L1 | EVQLVESGGGLVQPGGSLRLSCAASGFTFSRYW |
| | | | MSWVRQAPGKGLEWVANIKQDGSEKYYVDSVK |
| | | | GRFTISRDNAKNSLYLQMNSLRAEDTAVYYCARE |
| | | | GGWFGELAFDYWGQGTLVTVSSASTKGPSVFPL |
| | | | APSSKSTSGGTAALGCLVKDYFPEPVTVSWNSG |
| | | | ALTSGVHTFPAVLQSSGLYSLSSVVTVPSSSLGT |
| | | | QTYICNVNHKPSNTKVDKKVEPKSCDKTHTGGS |
| | | | SGSGSGSTGTSSSGTGTSAGTTGTSASTSGSGS |
| | | | GGGGGSGGGGSAGGEIVLTQSPGTLSLSPGERA |
| | | | TLSCRASQRVSSSYLAWYQQKPGQAPRLLIYDA |
| | | | SSRATGIPDRFSGSGSGTDFTLTISRLEPEDFAVY |
| | | | YCQQYGSLPWTFGQGTKVEIKRTVAAPSVFIFPP |
| | | | SDEQLKSGTASVVCLLNNFYPREAKVQWKVDNA |
| | | | LQSGNSQESVTEQDSKDSTYSLSSTLTLSKADYE |
| | | | KHKVYACEVTHQGLSSPVTKSFNRGEC |

### LEGENDS OF THE FIGURES

FIG. 1: Schematic representation of bispecific antibodies according to the format IgG-scFv.

FIG. 2: Binding of bsAbs and parental mAbs to membrane-displayed receptors assessed by flow cytometry. A. Representative flow cytometry profiles for binding of ICT01 versus its corresponding isotype and anti-BTN3AxPD-1 bsAbs (for example: bsAb001) on HEK-BTN3A1 at 10 and 100 nM. B. Representative flow cytometry profiles for binding of Pembrolizumab (Pembro) versus its corresponding isotype and anti-BTN3AxPD-1 bsAbs (for example: bsAb001) on Jurkat-PD-1 cells. C. Representative flow cytometry profiles for binding of Durvalumab versus its corresponding isotype and anti-BTN3AxPD-L1 bsAbs (for example: bsAb003) on CHO-PD-L1 cells.

FIG. 3: Impact of bsAbs and parental mAbs on Vγ9Vδ2 TCR and γβ TCR activation in cell-based reporter assays. A. Representative flow cytometry profiles for Vγ9Vδ2 TCR expression on Jurkat-Vγ9Vδ2 TCR MOP cells and BTN3A expression on Raji cells. B. Activation of Jurkat-Vγ9Vδ2 TCR MOP cells as determined by luminescence after co-culture of Jurkat-Vγ9Vδ2 TCR MOP with Raji cells in presence of ICT01 versus its corresponding isotype control or anti-BTN3AxPD-1 bsAbs (for example bsAb001 and bsAb002) or anti-BTN3AxPD-L1 bsAbs (for example bsAb003 and bsAb004), used at 0.5 and 5 nM. Data from two replicates are represented. C. Representative flow cytometry profiles for surface PD-1 and PD-L1 expression on Jurkat-PD-1 and on Raji-PD-L1 cells respectively. D. Activation of Jurkat-PD-1 cells assessed by luminescence following co-culture with SED-preincubated Raji-PD-L1 cells in the presence of Pembrolizumab or Durvalumab versus their corresponding isotype controls or anti-BTN3AxPD-1 bsAbs (for example bsAb001) or anti-BTN3AxPD-L1 bsAbs (for example bsAb003) used at 10 and 100 nM. Data from two replicates are represented.

FIG. 4: Dose-dependent effect of bsAbs on Vγ9Vδ2 TCR activation of Jurkat-Vγ9Vδ2 TCR MOP cells. A. Dose-dependent effect of ICT01 vs bsAbs, for example bsAb001 (anti-BTN3AxPD-1) and bsAb003 (anti-BTN3AxPD-L1) being in the same format, on Vγ9Vδ2 TCR activation in a Jurkat-Vγ9Vδ2 TCR MOP reporter assay. Data from two replicates are represented. Curve fitting is obtained using log (agonist) vs. response variable slope (four parameters). B. Dose-dependent effect of the 23 selected bsAbs (10 anti-BTN3AxPD-1 and 13 anti-BTN3AxPD-L1) in comparison with ICT01, on Vγ9Vδ2 TCR activation in a Jurkat-Vγ9Vδ2 TCR MOP reporter assay. The mean fold-changes in luminescence values relative to the non-treated condition (Medium) are represented in the heatmap.

FIG. 5: Dose-dependent effect of bsAbs on Vγ9Vδ2 T cell degranulation against ovarian cancer cells (SKOV3 cell line). A. Representative flow cytometry profiles for BTN3A expression on SKOV3 cells. B. Dose-dependent effect of ICT01 vs bsAbs, for example bsAb001 (anti-BTN3AxPD-1) and bsAb003 (anti-BTN3AxPD-L1) being in the same format, on Vγ9Vδ2 T cell degranulation in co-culture with SKOV3 cells. Data from 3 donors are represented as mean±standard error of the mean (SEM). C. Dose-dependent effect of the 23 selected bsAbs (10 anti-BTN3AxPD-1 and 13 anti-BTN3AxPD-L1) on Vγ9Vδ2 T cell degranulation (n=3). The mean fold-changes in the % CD107ab+ on Vγ9Vδ2 T cells relative to the non-treated condition (Medium) are represented in the heatmap. D. The activities of two reference bsAbs (bsAb001 and bsAb003) on Vγ9Vδ2 T cell degranulation compared to ICT01 at 0.1 nM across five individual donors, represented as mean±SEM. Paired t-tests were used to compare paired groups. * p<0.05) and *** p<0.001.

FIG. 6: Dose-dependent effect of bsAbs on expanded Vγ9Vδ2 T cell-mediated killing of ovarian cancer cells (SKOV3). A. Representative flow cytometry profiles for PD-1, PD-L1 and BTN3A expression on expanded Vγ9Vδ2 T cells and SKOV3 target cells. B. Dose-dependent effect of ICT01 vs bsAb003 (anti-BTN3AxPD-L1) on Vγ9Vδ2 T cell-mediated killing of SKOV3 target cells over 7 days. AUC values from 3 donors are represented as mean±SEM and curve fitting is obtained using log (agonist) vs. response_variable slope (four parameters). C. Effect of 13 bsAbs on Vγ9Vδ2 T cell-mediated killing of SKOV3 target cells over 7 days (n=3). The mean fold-changes of AUC values relative to the non-treated condition (Medium) are represented in the heatmap. D. Activity of 4 reference anti-BTN3AxPD-L1 bsAbs on Vγ9Vδ2 T cell-mediated killing of SKOV3 target cells compared to ICT01 at 0.01 nM across six individual donors monitored over 6 days, represented as mean±SEM. Paired t-tests were used to compare paired groups. **** p<0.0001.

FIG. 7: Impact of bsAb design on the anti-BTN3AxPD-L1 bsAbs-mediated Vγ9Vδ2 T cell functions. A. Illustrative pictures for four anti-BTN3AxPD-L1 bsAbs, in which the scFv of anti-PD-L1 is fused to either the N-terminus of the heavy chain (bsAb019 and bsAb023) or the light chain (bsAb063 and bsAb064) of anti-BTN3A (ICT01). B. Dose-dependent effect of four anti-BTN3AxPD-L1 bsAbs (bsAb019, bsAb023, bsAb063 and bsAb064) on Jurkat Vγ9Vδ2 TCR MOP activation in coculture with Raji cells. Data from two replicates are represented. C. Dose-dependent effect of bsAb019, bsAb023, bsAb063 and bsAb064 on Vγ9Vδ2 T cell degranulation against SKOV3 cells. Means±SEM of 3 donors are represented. D. Dose-dependent effect of bsAb019, bsAb023, bsAb063 and bsAb064 on Vγ9Vδ2 T cell-mediated killing of SKOV3 cells. The mean fold-changes of AUC values relative to the non-treated condition (Medium) of 3 donors are represented in the heatmap.

FIG. 8: Impact of the bsAbs on mediating IFN-γ secretion in a MLR assay compared to the combination of ICT01 and Durvalumab. A. Dose-dependent effect of ICT01 or Durvalumab as single agents or in combination, on IFN-γ production by T cells in a co-culture with allogeneic moDC. Data from 11 donors are represented as mean±SEM. Two-way ANOVA and Holm-Sidak's multiple comparisons test were used for comparing two independent variables. *** p<0.001 and **** p<0.0001. B. Dose-dependent effect of the reference anti-BTN3AxPD-L1 bsAb (bsAb003) versus ICT01+Durvalumab combination on IFN-γ production by T cells in a co-culture with allogeneic moDC (n=11). Wilcoxon tests were used for comparing two paired groups. * p<0.05 and ** p<0.01.

### EXAMPLES

### Material & Methods

1. bsAbs Generation

Human VH and VK sequences were synthesized in vitro and amplified by PCR using HiFi Polymerase. PCR products were cloned in human gamma1 heavy chain and kappa light chain expression vectors, and plasmids were transformed into competent bacteria. Vector sequencing was performed to validate the bsAbs cloning, before large scale (midi) preparation of plasmid for further transfection. Vectors encoding matched light and heavy chains for bsAbs were transiently transfected in CHO cells. Affinity purification of antibodies was performed with HiTrap MabSelect PrismA. Binding buffer was PBS, 850 mM NaCl. Elution was performed with the following buffer: 0.1 M Na-citrate pH 3.3. Samples were neutralized right after elution with 1 M Tris-HCl, pH 9 (10% v/v). Purity, as defined by the fraction of monomeric bsAbs, was determined by HPLC-SEC. SDS-PAGE of purified products was performed to verify the fragmentation and/or aggregation status of the final material. Endotoxin level was determined using a Endosafe® nexgen-PTS™ system (Charles River Endosafe).

Control antibodies Durvalumab and Pembrolizumab were generated, produced and purified similarly to the bsAbs, but using HEK cells. Binding buffer was Glycine 0.5 M, NaCl 1 M, pH 9, and elution was performed with Glycine 0.1 M pH 3.

ICT01 was produced in CHO cells using similar procedures.

### 2. Binding to Recombinant Proteins

Affinity measurements with respect to BTN3A, PD-1 and PD-L1 were performed by surface plasmon resonance (SPR), in a Carterra LSA Platform instrument with an anti-Fc functionalized HC30M SPR chip using a soluble form of the antigen as the ligand and the antibody as the analyte.

SPR measures the interactions between two or more biomolecules. In capture kinetics analysis, an antibody is immobilized onto a gold-plated prism. The immobilized antibody is referred to as the ligand. An injection of antigen, referred to as the analyte, is carried out, often as a concentration series. The captured analytes are removed from the sensor chip surface using a regenerating agent, for example a low pH or high ionic strength solution. If investigating binding to several analytes, the antibodies are recaptured from solution before each analyte titration series. The instrument records the changes in refractive indexes (due to the accumulation or depletion of mass) in time and plots the results as sensorgrams.

The collected data was analysed using Carterra Kinetics software version 1.8.1.3828.

### 3. In Cellulo Binding

The ability of bsAbs to bind membrane receptors (BTN3A1, PD-1 and/or PD-L1) was determined by flow cytometry using stably transduced cells. The cell lines used in the binding assay were generated by lentiviral transduction as described in the table below:

| | | | | Stable cell |
|---|---|---|---|---|
| Target | Cell line | Description | Culture medium | selection |
| BTN3A | HEK- | HEK 293T | DMEM GlutaMAX ™ | 1 μg/mL |
| | BTN3A1 | (ATCC®) - | Medium containing | Puromycin |
| | | BTN2A/BTN3A KO | 10% fetal bovine serum | |
| | | cells transduced | (FBS) and 1 mM | |
| | | with BTN3A1 | Sodium Pyruvate | |
| | | expressing vector | (NaPyr) | |
| PD-1 | Jurkat-PD-1 | Jurkat cells | RPMI GlutaMAX ™ | 1 μg/mL |
| | | (ATCC®) | Medium containing | Puromycin |
| | | transduced with | 10% FBS and 1 mM | |
| | | PD-1 expressing | NaPyr | |
| | | vector | | |
| PD-L1 | CHO-PD-L1 | CHO-TCRactivator | Ham's F12 Nut | 2 μg/mL |
| | | cells (BPS | GlutaMAX ™ Medium | Puromycin + |
| | | bioscience) | containing 10% FBS | 500 μg/mL |
| | | transduced with | and 1 mM NaPyr | Hygromycin B |
| | | PD-L1 expressing | | |
| | | vector | | |

For binding assays, confluent cells were harvested, washed with complete medium, then resuspended in FACS buffer (PBS, 1% FBS, 2 mM ethylenediaminetetraacetic acid (EDTA)) and transferred into a 96 well U bottom plate at a density of 10⁵ cells/well. Cells were stained for 30 minutes at 4° C. with 10 and 100 nM of bsAbs or positive controls (ICT01 or Pembrolizumab or Durvalumab) vs corresponding isotype controls prepared in FACS buffer. After extensive wash with FACS buffer, 50 μL of mix containing a viability marker (live/dead near IR, Thermofisher Scientific) and a PE-conjugated secondary antibody (Jackson ImmunoResearch Laboratories) diluted 1/200 in FACS buffer were added for 20 minutes at 4° C. Cells were washed and resuspended in diluted fixation buffer (BD Biosciences) before acquisition on a Cytoflex LX instrument (Beckman Coulter).

Flow cytometry data were analysed using FlowJo® Software. Median fluorescence intensity (MFI) values were reported.

### 4. PD-1, PD-L1 and BTN3A Expression on Expanded Vγ9Vδ2 T Cells and Tumor Cell Lines

Assessment of PD-1, PD-L1 and BTN3A surface expression on tumor cell lines including Jurkat-MOP, Jurkat-PD-1, Raji and SKOV3, was performed by flow cytometry. Briefly, confluent cells were harvested and washed with FACS buffer then incubated 10 minutes at room temperature with FcR blocking Reagent (Miltenyi Biotec) before staining with conjugated mAbs (anti-PD-1-BV421, anti-PD-L1 PC7, anti-BTN3A AF546) and a viability marker (LIVE/DEAD™ near IR) for 20 minutes at 4° C. Each sample was also stained with a second mix of mAbs containing the respective isotype controls for anti-PD-1, anti-PD-L1 and anti-BTN3A mAbs to allow proper gating. After incubation, stained cells were washed twice in FACS buffer before acquisition on a Cytoflex LX instrument (Beckman Coulter).

In vitro expanded Vγ9Vδ2 T cells were also analysed for PD-1, PD-L1 and BTN3A expression by flow cytometry using conjugated mAbs (anti-CD3 PE CF594, anti-Vd2TCR FITC, anti-PD-1-BV421, anti-PD-L1 PC7, anti-BTN3A AF546) and a viability marker (LIVE/DEAD™ near IR) using the same staining procedure as described above. A second mix of mAbs containing the respective isotype controls for anti-PD-1, anti-PD-L1 and anti-BTN3A mAbs was used in parallel. Flow cytometry data were analysed using FlowJo® Software. Median fluorescence intensity (MFI) values were reported.

### 5. Cell-Based Reporter Assays to Evaluate BTN3A Agonist Activity on Vγ9Vδ2 TCR Activation and PD-1/PD-L1 Blockade Activity on γβ TCR Activation.

Jurkat (human acute T cell leukemia, ATCC®) cells were stably transduced to express the Vγ9Vδ2 TCR (clone MOP) and a NFAT reporter (Jurkat-Vγ9V82 TCR MOP), or PD-1 and a NFAT reporter (Jurkat-PD-1). Raji cells (human Burkitt's lymphoma, ATCC®) were engineered to express PD-L1 (Raji-PD-L1) using lentiviral transduction. Selection of stable cells was performed by supplementing the culture medium with 1 μg/mL puromycin as outlined in the table below:

| | | Stable cell |
|---|---|---|
| Cell line | Culture medium | selection |
| Jurkat-Vy9V02 | RPMI GlutaMAX ™ Medium | 1 μg/mL |
| TCR MOP cells | containing 10% FBS and | Puromycin |
| | 1 mM NaPyr | |
| Jurkat PD-1 | RPMI GlutaMAX ™ Medium | 1 μg/mL |
| cells | containing 10% FBS and | Puromycin |
| | 1 mM NaPyr | |
| Raji-PD-L1 | RPMI GlutaMAX ™ Medium | 1 μg/mL |
| cells | containing 10% FBS and | Puromycin |
| | 1 mM NaPyr | |
| Raji cells | RPMI GlutaMAX ™ Medium | Non applicable |
| | containing 10% FBS and | |
| | 1 mM NaPyr | |

To assess the agonist activity of the anti-BTN3A moiety of the constructs, Raji cells were first incubated for 10 minutes at room temperature in PBS/10% FCR blocking solution (Miltenyi). Then, 75×10³ Raji cells and 25×10³ Jurkat-Vγ9Vδ2 TCR MOP cells (ratio 3:1) were co-cultured overnight at 37° C., 5% CO₂ in 96-well U bottom plates in presence of bsAbs or ICT01 versus corresponding isotype control at different doses. PMA/lonomycin (Sigma) was used as a positive control of Jurkat-Vγ9Vδ2 TCR MOP activation. After incubation, plates were centrifuged and supernatants were harvested and transferred into a new 96-well plate. A volume of 20 μL of supernatants was transferred in 96-well flat bottom white plates for luciferase quantification using QuantiLuc reagent (Invivogen) according to manufacturer's instructions. Luminescence was measured using TECAN Spark instrument. Raw data were exported to Excel files and plotted using GraphPad PRISM software. Curve fitting was obtained using log (agonist) vs. response_variable slope (four parameters) from GraphPad Prism software.

For PD-1/PD-L1 blockade activity assessment, Raji-PD-L1 cells were first incubated for 10 minutes at room temperature in PBS/10% FCR blocking solution and then preincubated with 0.1 μg/mL of Staphylococcal enterotoxin D (SED, Toxin technologies) for 30 minutes. SED-preincubated Raji-PD-L1 cells (30×10³) were co-cultured with 120×10³ Jurkat-PD-1 cells (ratio 1:4) overnight at 37° C., 5% CO₂ in 96-well U bottom plates in presence of bsAbs or Pembrolizumab or Durvalumab versus corresponding isotype controls at different doses. PMA/lonomycin (Sigma) was used as control for Jurkat-PD-1 activation. After incubation, plates were centrifuged and 20 μL of supernatants were transferred in 96-well flat bottom white plates for luciferase quantification as described above.

### 6. Vγ9Vδ2 T Cell Degranulation and Killing Assay Against an Ovarian Cancer Cell Line (SKOV3).

Human (Hu) EDTA-buffy coats were obtained from the Etablissement Français du Sang (EFS) Provence Alpes Côthe d′Azur (France) under the agreement n°7173. Peripheral blood mononuclear cells (PBMC) were isolated by Ficoll density gradient centrifugation of EDTA-buffy coats.

To expand Vγ9Vδ2 T cells for degranulation and killing assay, healthy donor (HD) hu-PBMC were resuspended in complete medium (RPMI 1640 medium supplemented with 10% FBS, 1% NaPyr and 1% Penicillin/streptomycin (P/S)) for 12-13 days in the presence of recombinant human Interleukin-2 (rhIL-2 (ProLeukin®, Novartis) (200 International Units (IU)/mL) and amino-bisphosphonates (Zoledronate (Sigma-Aldrich), 1 μM). From day 5, rhIL-2 was replenished every 2 or 3 days by adding fresh complete culture medium supplemented with rhIL-2 and cells were kept at a concentration of 10⁶ cells/mL. Expanded Vγ9Vδ2 T cells were collected at day 12-13 for phenotyping and killing assays.

SKOV3 cell line (human ovarian adenocarcinoma) was purchased from the European Collection of Authenticated Cell Cultures (ECACC) and cultured at 37° C. and 5% CO₂ in McCoy's Glutamax medium supplemented with 10% FBS and 1 nM NaPyr. mKate-expressing SKOV3 cells were established by lentiviral transduction with the IncuCyte® NucLight Red Lentivirus Reagent (Sartorius) at a multiplicity of infection of 3 in the presence of 8 μg/mL of Polybrene® (Sigma-Aldrich). Selection of transduced cells was performed by adding 1 μg/mL puromycin (Invivogen) to the culture. Cells were tested for mycoplasma contamination by PCR using the MycoplasmaCheck platform (Eurofins).

For assessment of Vγ9Vδ2 T cell degranulation against tumor cells, 104 expanded Vγ9Vδ2 T cells were cultured with an equal number of SKOV3 cells for 4 hours in the presence of a mix of anti-CD107a+b mAbs and Golgi stop (BD Biosciences). When indicated, cells were treated with bsAbs or ICT01 versus corresponding isotype control at different doses. After 4 hours of culture, cells were washed and incubated with a mix of anti-CD3, anti-Vδ2 TCR mAb and a viability marker (LIVE/DEAD™ Fixable) for 20 minutes at 4° C. After incubation, cells were washed twice in FACS buffer and were then resuspended in Cytofix solution before analysis on a CytoFLEX LX flow cytometer (Beckman Coulter)

For the killing assays evaluating the effect of the bsAbs on the cytolytic activity of expanded Vγ9Vδ2 T cells, 2.5×10³ mkate SKOV3 cells were seeded in 96-well plates and cultured overnight in appropriate complete medium. The day of the experiment, 2.5×10³ in vitro expanded Vγ9Vδ2 T cells were added and cultured for 6-7 days in the presence of anti-BTN3AxPD-L1 bsAbs or parental mAbs (ICT01 or Durvalumab) versus corresponding isotype controls at different doses. Wells containing target cells only were used as controls of tumor cell growth and viability. The total number of tumor target cells was followed over time by measuring the fluorescent signal emitted by the NucLight red dye using the Incucyte® live cell imaging system. Tumor cell growth represented by the tumor cell count was calculated for each well and normalized to baseline (corresponding to the number of tumor cells present in the well 6 hours after the addition of expanded Vγ9Vδ2 T cells). Data analysis was performed using GraphPad prism. To compare the activity between the different conditions, the AUC values (area under the curve) were reported. Additionally, fold-change of AUC relative to the non-treated condition were calculated for each dose and each donor.

### 7. Mixed Lymphocyte Reaction (MLR) Assay

CD14⁺ monocytes and CD3⁺ T cells were isolated from hu-PBMC by positive and negative selection using human CD14 Microbeads (Miltenyi Biotec) and EasySep Human T cell Enrichment kit (Stemcell technologies), respectively.

moDC were generated by culturing CD14⁺ monocytes (10⁶/mL) in vitro for 5 days in RPMI 1640 GlutaMAX supplemented with 10% FBS, 1% P/S, 1 mM NaPyr, 1× HEPES, 1% MEM NEAA, in the presence of 100 ng/mL interleukin-4 (IL-4), Miltenyi) and 100 ng/ml GM-CSF (Miltenyi). On day 3, half of culture medium was replaced by fresh complete medium supplemented with IL-4 (100 ng/mL) and GM-CSF (100 ng/mL). After 5 days and prior to co-culture, moDCs were harvested using 1 mL of accutase diluted in PBS 2 mM EDTA. Phenotyping was performed to verify moDCs differentiation and maturation status by monitoring CD11b, CD1a, CD14, CD86, CD83 and HLA-DR expression by flow cytometry.

For MLR assay, 105 isolated CD3+ T cells were co-cultured with 104 allogeneic moDCs (at a 10:1 ratio) in RPMI 1640 medium supplemented with 10% FBS, 1 mM NaPyr for 3 days with or without the reference bsAb003 at different doses. ICT01 or Durvalumab used as single agents or in combination were added as controls. Co-culture was done in 96-well round bottom plates in a final volume of 200 μL. After 3 days of incubation, culture supernatants were collected for IFN-γ quantification by ELISA using a human IFN-γ uncoated ELISA kit (ThermoFisher Scientific). Data were plotted and analyzed using GraphPad PRISM software.

### 8. Statistics

Two-way ANOVA and Holm-Sidak's multiple comparison tests were employed to assess the impact of two independent variables on a continuous dependent variable. Paired t-tests were used to compare two paired groups with normally distributed data, whereas Wilcoxon rank-sum test was employed for comparing two paired groups when the data were not normally distributed. Curve fitting was obtained using log (agonist) vs. response_variable slope (four parameters). Statistical analyses were conducted using GraphPad Prism 9.0 (GraphPad Software). Significance was determined when the p-value was below 0.05 denoted as * (p<0.05), ** (p<0.01), *** (p<0.001), and **** (p<0.0001)

### Affinity Measurements

Affinity measurements with respect to BTN3A, PD-1 and PD-L1 were performed by SPR technology, for instance in a Carterra LSA Platform instrument using a soluble form of the antigen as the analyte and the antibody as the ligand. Binding was also assessed through in cellulo binding assays, on cell lines (e.g. HEK293T cells) genetically modified to express the targets of interest or genetically modified to extinct target expression.

### Reporter Assay

Human Jurkat cells expressing NFAT-induced luciferase with constitutive expression of Vγ9Vδ2 TCR MOP and human PD-1 (Jurkat-NFAT-MOP-PD-1) were co-cultured with Raji cells stably expressing PD-L1 (Raji-PD-L1) in presence or absence of the bispecific constructs at different doses or their corresponding controls. The co-culture was performed in RPMI medium supplemented with GlutaMax, 10% FBS, 1 mM NaPy for an overnight incubation at 37° C. 5% CO₂.

Anti-BTN3A agonist mAb (ICT01) or anti-PD-1 mAb (Pembrolizumab) or anti-PD-L1 mAb (Durvalumab) or combination of anti-BTN3A with anti-PD-1 or anti-PD-L1 mAbs were used as comparator and their corresponding isotype controls were used as negative control. Jurkat-NFAT-MOP-PD-1 reporter cells alone are incubated with PMA (20 ng/ml)/lonomycin (1 μg/mL) (Sigma) as positive control. At the end of the incubation, the activation of the luciferase gene in the Jurkat-NFAT-MOP-PD-1 reporter cells was measured by luminescence using QuantiLuc reagent (Invivogen).

In a further assay, human Jurkat-NFAT cells expressing human Vγ9Vδ2 TCR MOP (Jurkat-NFAT-MOP) or Jurkat-NFAT cells expressing human PD-1 (Jurkat-NFAT-PD-1) were used to monitor activity of bispecific constructs. Briefly, Jurkat-NFAT-MOP and Jurkat-NFAT-PD-1 cells were co-cultured with Raji or Raji-PD-L1 cells in presence of the bispecific constructs or their corresponding controls using the same protocol as described above. For co-culture of Jurkat-NFAT-PD-1 cells with Raji-PD-L1 cells, γβ TCR activating reagent (such as superantigen) was added in the co-culture to induce Jurkat activation.

### Example 1: Antibody Designs and Sequences

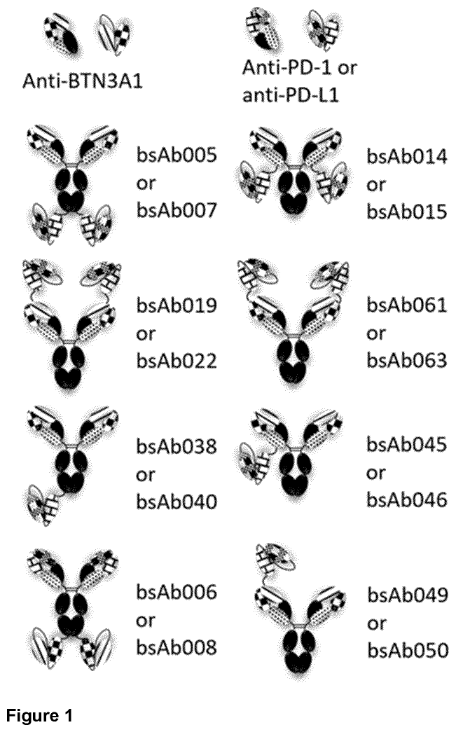

IgG-scFv antibodies were designed according to the construct designs shown in FIG. 1, as listed in Table 2 below, i.e. based on an anti-BTN3A IgG linked to anti-PD-1 or anti-PD-L1 antibodies. The anti-BTN3A IgG corresponds to mAb 7.2 as disclosed in WO2020/025703, the anti-PD-1 scFv is based on the VH/VL sequences of pembrolizumab and the anti-PD-L1 scFv is based on the VH/VL sequences of durvalumab. Amino acid sequences for the anti-PD-1/PD-L1 VH and VL sequences, the anti-BTN3A VH and VL sequences, CL, CH1, CH2 and CH3 sequences are specified in Table 1. The linker GGGGSGGGGSGGGGSGGGGS was used to link the scFv to the C-terminus or N-terminus of the heavy or light chain(s) of the IgG.

TABLE 2
Design of the bispecific constructs

| | | scFv or | | | | |
|---|---|---|---|---|---|---|
| Antibody | IgG | scFab | | | | |
| n° | specificity | specificity | Heavy chain 1 | Heavy chain 2 | Light chain 1 | Light chain 2 |
| 1 | BTN3A | PD-1 | IgG(H)-linker1- | IgG(H)-linker1- | IgG(L) | IgG(L) |
| | | | VH-linker2-VL | VH-linker2-VL | | |
| 2 | PD-1 | BTN3A | IgG(H)-linker1- | IgG(H)-linker1- | IgG(L) | IgG(L) |
| | | | VH-linker2-VL | VH-linker2-VL | | |
| 3 | BTN3A | PD-L1 | IgG(H)-linker1- | IgG(H)-linker1- | IgG(L) | IgG(L) |
| | | | VH-linker2-VL | VH-linker2-VL | | |
| 4 | PD-L1 | BTN3A | IgG(H)-linker1- | IgG(H)-linker1- | IgG(L) | IgG(L) |
| | | | VH-linker2-VL | VH-linker2-VL | | |
| 5 | BTN3A | PD-1 | IgG(H)-linker1- | IgG(H)-linker1- | IgG(L) | IgG(L) |
| | | | VL-linker2-VH | VL-linker2-VH | | |
| 6 | PD-1 | BTN3A | IgG(H)-linker1- | IgG(H)-linker1- | IgG(L) | IgG(L) |
| | | | VL-linker2-VH | VL-linker2-VH | | |
| 7 | BTN3A | PD-L1 | IgG(H)-linker1- | IgG(H)-linker1- | IgG(L) | IgG(L) |
| | | | VL-linker2-VH | VL-linker2-VH | | |
| 8 | PD-L1 | BTN3A | IgG(H)-linker1- | IgG(H)-linker1- | IgG(L) | IgG(L) |
| | | | VL-linker2-VH | VL-linker2-VH | | |
| 9 | PD-1 | BTN3A | IgG-(H) | IgG-(H) | IgG(L)- | IgG(L)- |
| | | | | | linker1-VH- | linker1-VH- |
| | | | | | linker2-VL | linker2-VL |
| 10 | BTN3A | PD-1 | IgG-(H) | IgG-(H) | IgG(L)- | IgG(L)- |
| | | | | | linker1-VH- | linker1-VH- |
| | | | | | linker2-VL | linker2-VL |
| 11 | BTN3A | PD-L1 | IgG-(H) | IgG-(H) | IgG(L)- | IgG(L)- |
| | | | | | linker1-VH- | linker1-VH- |
| | | | | | linker2-VL | linker2-VL |
| 12 | PD-L1 | BTN3A | IgG-(H) | IgG-(H) | IgG(L)- | IgG(L)- |
| | | | | | linker1-VH- | linker1-VH- |
| | | | | | linker2-VL | linker2-VL |
| 13 | PD-1 | BTN3A | IgG-(H) | IgG-(H) | IgG(L)- | IgG(L)- |
| | | | | | linker1-VL- | linker1-VL- |
| | | | | | linker2-VH | linker2-VH |
| 14 | BTN3A | PD-1 | IgG-(H) | IgG-(H) | IgG(L)- | IgG(L)- |
| | | | | | linker1-VL- | linker1-VL- |
| | | | | | linker2-VH | linker2-VH |
| 15 | BTN3A | PD-L1 | IgG-(H) | IgG-(H) | IgG(L)- | IgG(L)- |
| | | | | | linker1-VL- | linker1-VL- |
| | | | | | linker2-VH | linker2-VH |
| 16 | PD-L1 | BTN3A | IgG-(H) | IgG-(H) | IgG(L)- | IgG(L)- |
| | | | | | linker1-VL- | linker1-VL- |
| | | | | | linker2-VH | linker2-VH |
| 17 | PD-1 | BTN3A | VL-linker1-VH- | VL-linker1-VH- | IgG(L) | IgG(L) |
| | | | linker2-IgG(H) | linker2-IgG(H) | | |
| 18 | BTN3A | PD-1 | VL-linker1-VH- | VL-linker1-VH- | IgG(L) | IgG(L) |
| | | | linker2-IgG(H) | linker2-IgG(H) | | |
| 19 | 3TN3A | PD-L1 | VH-linker1-VL- | VH-linker1-VL- | IgG(L) | IgG(L) |
| | | | linker2-IgG(H) | inker2-IgG(H) | | |
| 20 | PD-L1 | BTN3A | VL-linker1-VH- | VL-linker1-VH- | IgG(L) | IgG(L) |
| | | | linker2-IgG(H) | linker2-IgG(H) | | |
| 21 | PD-1 | BTN3A | VH-linker1-VL- | VH-linker1-VL- | IgG(L) | IgG(L) |
| | | | linker2-IgG(H) | linker2-IgG(H) | | |
| 22 | BTN3A | PD-1 | VH-linker1-VL- | VH-linker1-VL- | IgG(L) | IgG(L) |
| | | | linker2-IgG(H) | linker2-IgG(H) | | |
| 23 | BTN3A | PD-L1 | VL-linker1-VH- | VL-linker1-VH- | IgG(L) | IgG(L) |
| | | | linker2-IgG(H) | linker2-IgG(H) | | |
| 24 | PD-L1 | BTN3A | VH-linker1-VL- | VH-linker1-VL- | IgG(L) | IgG(L) |
| | | | linker2-IgG(H) | linker2-IgG(H) | | |
| 25 | BTN3A | PD-1 | VH-CH1-linker1- | VH-CH1- | IgG(L) | IgG(L) |
| | | | VL-CL-linker2- | linker1-VL-CL- | | |
| | | | IgG(H) | linker2-IgG(H) | | |
| 26 | BTN3A | PD-1 | IgG(H)-VH-CH1- | IgG(H)-VH- | IgG(L) | IgG(L) |
| | | | linker1-VL-CL- | CH1-linker1- | | |
| | | | linker2- | VL-CL-linker2- | | |
| 27 | BTN3A | PD-L1 | VH-CH1-linker1- | VH-CH1- | IgG(L) | IgG(L) |
| | | | VL-CL-linker2- | linker1-VL-CL- | | |
| | | | IgG(H) | linker2-IgG(H) | | |
| 28 | BTN3A | PD-L1 | IgG(H)-VH-CH1- | IgG(H)-VH- | IgG(L) | IgG(L) |
| | | | linker1-VL-CL- | CH1-linker1- | | |
| | | | linker2- | VL-CL-linker2- | | |
| 29 | PD-L1 | BTN3A | VH-CH1-linker1- | VH-CH1- | IgG(L) | IgG(L) |
| | | | VL-CL-linker2- | linker1-VL-CL- | | |
| | | | IgG(H) | linker2-IgG(H) | | |
| 30 | PD-L1 | BTN3A | IgG(H)-VH-CH1- | IgG(H)-VH- | IgG(L) | IgG(L) |
| | | | linker1-VL-CL- | CH1-linker1- | | |
| | | | linker2- | VL-CL-linker2- | | |
| 31 | PD-1 | BTN3A | VH-CH1-linker1- | VH-CH1- | IgG(L) | IgG(L) |
| | | | VL-CL-linker2- | linker1-VL-CL- | | |
| | | | IgG(H) | linker2-IgG(H) | | |
| 32 | PD-1 | BTN3A | IgG(H)-VH-CH1- | IgG(H)-VH- | IgG(L) | IgG(L) |
| | | | linker1-VL-CL- | CH1-linker1- | | |
| | | | linker2- | VL-CL-linker2- | | |
| 33 | BTN3A | PD-1 | IgG(H)-linker1- | IgG-(H) | IgG(L) | IgG(L) |
| | | | VH-linker2-VL | | | |
| 34 | PD-1 | BTN3A | IgG(H)-linker1- | CH2-CH3- | IgG(L) | none |
| | | | VH-linker2-VL | linker1-VH- | | |
| | | | | linker2-VL | | |
| 35 | BTN3A | PD-L1 | IgG(H)-linker1- | IgG-(H) | IgG(L) | IgG(L) |
| | | | VH-linker2-VL | | | |
| 36 | PD-L1 | BTN3A | IgG(H)-linker1- | CH2-CH3- | IgG(L) | none |
| | | | VH-linker2-VL | linker1-VH- | | |
| | | | | linker2-VL | | |
| 37 | PD-1 | BTN3A | IgG-(H) | VL-linker1-VH- | IgG(L)- | none |
| | | | | linker2-CH2- | linker1-VH- | |
| | | | | CH3 | linker2-VL | |
| 38 | BTN3A | PD-1 | IgG(H)-linker1- | IgG-(H) | IgG(L) | IgG(L) |
| | | | VL-linker2-VH | | | |
| 39 | PD-1 | BTN3A | IgG(H)-linker1- | CH2-CH3- | IgG(L) | none |
| | | | VL-linker2-VH | linker1-VL- | | |
| | | | | linker2-VH | | |
| 40 | BTN3A | PD-L1 | IgG(H)-linker1- | IgG-(H) | IgG(L) | IgG(L) |
| | | | VL-linker2-VH | | | |
| 41 | PD-L1 | BTN3A | IgG(H)-linker1- | CH2-CH3- | IgG(L) | none |
| | | | VL-linker2-VH | linker1-VL- | | |
| | | | | linker2-VH | | |
| 42 | PD-1 | BTN3A | IgG-(H) | VH-linker1-VL- | IgG(L)- | none |
| | | | | linker2-CH2- | linker1-VL- | |
| | | | | CH3 | linker2-VH | |
| 43 | BTN3A | PD-1 | IgG-(H) | IgG-(H) | IgG(L)- | IgG(L) |
| | | | | | linker1-VH- | |
| | | | | | linker2-VL | |
| 44 | BTN3A | PD-L1 | IgG-(H) | IgG-(H) | IgG(L)- | IgG(L) |
| | | | | | linker1-VH- | |
| | | | | | linker2-VL | |
| 45 | BTN3A | PD-1 | IgG-(H) | IgG-(H) | IgG(L)- | IgG(L) |
| | | | | | linker1-VL- | |
| | | | | | linker2-VH | |
| 46 | BTN3A | PD-L1 | IgG-(H) | IgG-(H) | IgG(L)- | IgG(L) |
| | | | | | linker1-VL- | |
| | | | | | linker2-VH | |
| 47 | BTN3A | PD-1 | VL-linker1-VH- | IgG-(H) | IgG(L) | IgG(L) |
| | | | linker2-IgG(H) | | | |
| 48 | BTN3A | PD-L1 | VL-linker1-VH- | IgG-(H) | IgG(L) | IgG(L) |
| | | | linker2-IgG(H) | | | |
| 49 | BTN3A | PD-1 | VH-linker1-VL- | IgG-(H) | IgG(L) | IgG(L) |
| | | | linker2-IgG(H) | | | |
| 50 | BTN3A | PD-L1 | VH-linker1-VL- | IgG-(H) | IgG(L) | IgG(L) |
| | | | linker2-IgG(H) | | | |
| 51 | BTN3A | PD-L1 | IgG(H)-linker1- | CH2-CH3- | IgG(L) | none |
| | | | VH-linker2-VL | linker1-VH- | | |
| | | | | linker2-VL | | |
| 52 | BTN3A | PD-1 | IgG(H)-linker1- | CH2-CH3- | IgG(L) | none |
| | | | VH-linker2-VL | linker1-VH- | | |
| | | | | linker2-VL | | |
| 53 | PD-1 | BTN3A | IgG(H)-linker1- | IgG-(H) | IgG(L) | IgG(L) |
| | | | VH-linker2-VL | | | |
| 54 | PD-L1 | BTN3A | IgG(H)-linker1- | IgG-(H) | IgG(L) | IgG(L) |
| | | | VH-linker2-VL | | | |
| 55 | BTN3A | PD-L1 | CH2-CH3- | IgG-(H) | none | IgG(L) |
| | | | linker1-VH- | | | |
| | | | linker2-VL | | | |
| 56 | BTN3A | PD-1 | CH2-CH3- | IgG-(H) | none | IgG(L) |
| | | | linker1-VH- | | | |
| | | | linker2-VL | | | |
| 57 | PD-L1 | BTN3A | IgG-(H) | IgG-(H) | VH-linker1-VL- | VH-linker1-VL- |
| | | | | | linker2-IgG(L) | linker2-IgG(L) |
| 58 | PD-L1 | BTN3A | IgG-(H) | IgG-(H) | VL-linker1-VH- | VL-linker1-VH- |
| | | | | | linker2-IgG(L) | linker2-IgG(L) |
| 59 | PD-1 | BTN3A | IgG-(H) | IgG-(H) | VH-linker1-VL- | VH-linker1-VL- |
| | | | | | linker2-IgG(L) | linker2-IgG(L) |
| 60 | PD-1 | BTN3A | IgG-(H) | IgG-(H) | VL-linker1-VH- | VL-linker1-VH- |
| | | | | | linker2-IgG(L) | linker2-IgG(L) |
| 61 | BTN3A | PD-1 | IgG-(H) | IgG-(H) | VH-linker1-VL- | VH-linker1-VL- |
| | | | | | linker2-IgG(L) | linker2-IgG(L) |
| 62 | BTN3A | PD-1 | IgG-(H) | IgG-(H) | VL-linker1-VH- | VL-linker1-VH- |
| | | | | | linker2-IgG(L) | linker2-IgG(L) |
| 63 | BTN3A | PD-L1 | IgG-(H) | IgG-(H) | VH-linker1-VL- | VH-linker1-VL- |
| | | | | | linker2-IgG(L) | linker2-IgG(L) |
| 64 | BTN3A | PD-L1 | IgG-(H) | IgG-(H) | VL-linker1-VH- | VL-linker1-VH- |
| | | | | | linker2-IgG(L) | linker2-IgG(L) |
| 65 | BTN3A | PD-L1 | IgG(H) | IgG(H) | VH-CH1- | VH-CH1- |
| | | | | | linker1-VL-CL- | linker1-VL-CL- |
| | | | | | linker2-IgG(L) | linker2-IgG(L) |
| 66 | PD-L1 | BTN3A | IgG(H) | IgG(H) | VH-CH1- | VH-CH1- |
| | | | | | linker1-VL-CL- | linker1-VL-CL- |
| | | | | | linker2-IgG(L) | linker2-IgG(L) |
| 67 | BTN3A | PD-1 | IgG(H) | IgG(H) | VH-CH1- | VH-CH1- |
| | | | | | linker1-VL-CL- | linker1-VL-CL- |
| | | | | | linker2-IgG(L) | linker2-IgG(L) |
| 68 | PD-1 | BTN3A | IgG(H) | IgG(H) | VH-CH1- | VH-CH1- |
| | | | | | linker1-VL-CL- | linker1-VL-CL- |
| | | | | | linker2-IgG(L) | linker2-IgG(L) |
| 69 | | | IgG-(H) | VL-linker1-VH- | IgG(L)- | none |
| | | | | linker2-CH2- | linker1-VH- | |
| | | | | CH3 | linker2-VL | |
| 70 | | | IgG-(H) | VH-linker1-VL- | IgG(L)- | none |
| | | | | linker2-CH2- | linker1-VL- | |
| | | | | CH3 | linker2-VH | |

### Example 2: Antibody Production

A total of 70 bsAbs in various formats (FIG. 1 and Table 2) and parental mAbs (ICT01, Pembrolizumab, Durvalumab versus corresponding isotype) were produced in CHO cells and purified using Protein A affinity chromatography. Sixty-nine out of the seventy bsAbs, comprising 34 anti-BTN3AxPD-1 and 35 anti-BTN3AxPD-L1 constructs, were successfully produced except for one construct, namely bsAb067. Human VH and VK bispecific mAb sequences were synthesized in vitro and amplified by PCR using HiFi Polymerase. PCR products were cloned in human IgG1 heavy chain and kappa light chain expression vectors, and plasmids were transformed into bacterial competent cells. Vector sequencing was performed to validate bispecific mAbs, before large scale (midi) preparation of plasmid for further transfection. Vectors encoding matched light and heavy chains for bispecific mAbs were transiently transfected in CHO cells. Affinity purification of antibodies was performed with HiTrap MabSelect PrismA. Binding buffer was PBS, 850 mM NaCl. Elution was performed with the following buffer: 0.1 M Na-citrate pH3.3. Samples were neutralized right after elution with 1M Tris-HCl, pH9 (10% v/v). Purity, as defined by the fraction of mAb monomer, was determined by HPLC-SEC. SDS-PAGE of purified antibody allowed detection of fragmentation and/or aggregation of the final material. Endotoxin level was determined using a Endosafe® nexgen-PTS™ system (Charles River Endosafe). Titer and purity of the produced antibodies were evaluated.

### Example 3: Evaluation of the Produced bsAbs Based on their Capacity to Modulate Vγ9Võ2 and γβ T Cell Activation

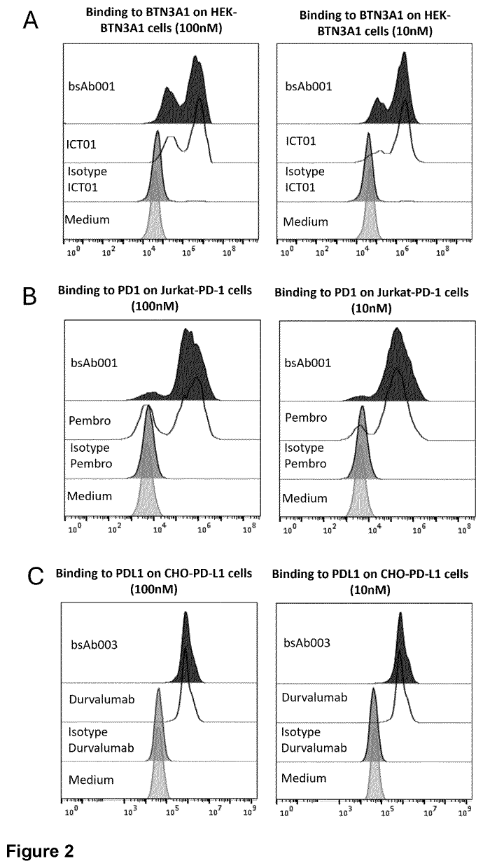

The binding ability of the 69 bsAbs and control parental mAbs (ICT01, Pembrolizumab, and Durvalumab), all at 10 and 100 nM, was assessed in cellulo by flow cytometry with cells expressing the target proteins. In this assay, all bsAbs demonstrated binding to their respective targets-BTN3A1, PD-1 and PD-L1 expressed at the surface of HEK-BTN3A1 cells, Jurkat-PD-1 cells and CHO-PD-L1 cells respectively, at both concentrations tested (FIG. 2 and Table 3).

The 69 produced bsAbs and control mAbs were also evaluated for their binding affinity to recombinant proteins by SPR assays. The equilibrium dissociation constant (K_{D}) was then calculated for constructs showing detectable binding. Results indicated that all 69 constructs bound to a recombinant BTN3A1 protein. However, some constructs failed to bind to recombinant PD-1 and PD-L1 proteins, leading to undetermined K_{D} values as shown in Table 3.

TABLE 3
Binding ability of 69 bsAbs to recombinant proteins and in cellulo

| | Binding to recombinant | | Binding in cellulo (Flow Cytometry) | | | |
|---|---|---|---|---|---|---|
| | proteins (SPR) | | HEK- | HEK- | Jurkat-PD- | Jurkat-PD- |
| | | rhPD-1 or | BTN3A1 | BTN3A1 | 1 or CHO- | 1 or CHO- |
| | rhBTN3A1 | rhPD-L1 | (MFI at | (MFI at | PD-L1 (MFI | PD-L1 (MFI |
| bsAbs and Controls | (K_{D}: nM) | (K_{D}: nM) | 10 nM) | 100 nM) | at 10 nM) | at 100 nM) |

Controls

| | | | | | | |
|---|---|---|---|---|---|---|
| Medium | NA | NA | 16253 | 14618 | 4872 | 5162 |
| Isotype controls | | | | | | |
| Isotype ICT01 | NA | NA | 31646 | 38119 | NA | NA |
| Isotype Pembrolizumab | NA | NA | NA | NA | 13548 | 5537 |
| Isotype Durvalumab | NA | NA | NA | NA | 38000 | 41594 |
| Positive controls | | | | | | |
| ICT01 | 0.073 | NA | 1011443 | 1821000 | NA | NA |
| Pembrolizumab | NA | 0.878 | NA | NA | 127126 | 228713 |
| Durvalumab | NA | 0.092 | NA | NA | 720000 | 756000 |

Anti-BTN3A × PD-1 constructs (n = 34)

| | | | | | | |
|---|---|---|---|---|---|---|
| Anti-BTN3A moiety, Fab | | | | | | |
| fragment (n = 18) | | | | | | |
| bsAb001 | 0.076 | 1.200 | 507000 | 710500 | 179119 | 395673 |
| bsAb005 | 0.076 | 2.100 | 490000 | 1105000 | 189967 | 351852 |
| bsAb010 | 0.121 | 1.700 | 636500 | 729500 | 271219 | 384355 |
| bsAb014 | 0.083 | 2.100 | 464464 | 811500 | 164805 | 289840 |
| bsAb018 | 0.212 | 1.200 | 502000 | 1075000 | 239663 | 347139 |
| bsAb022 | 0.163 | 0.928 | 224304 | 992000 | 215591 | 366625 |
| bsAb025 | 0.154 | 1.100 | 177385 | 669000 | 109692 | 358140 |
| bsAb026 | 0.068 | ND | 409542 | 1026500 | 73316 | 256616 |
| bsAb033 | 0.067 | 0.786 | 583500 | 1175000 | 194424 | 340434 |
| bsAb038 | 0.071 | 1.900 | 587500 | 1205000 | 159989 | 294330 |
| bsAb043 | 0.064 | ND | 559500 | 1054000 | 109573 | 357280 |
| bsAb045 | 0.059 | ND | 658500 | 941000 | 106563 | 270245 |
| bsAb047 | 0.168 | 1.400 | 535500 | 865500 | 263817 | 363582 |
| bsAb049 | 0.101 | 0.856 | 460500 | 740113 | 174654 | 375703 |
| bsAb052 | 0.069 | 1.800 | 321215 | 785000 | 185177 | 365681 |
| bsAb056 | 0.065 | 2.000 | 519000 | 1004500 | 218253 | 334377 |
| bsAb061 | 0.135 | 0.663 | 451000 | 1135000 | 216841 | 411235 |
| bsAb062 | 0.116 | 0.877 | 610500 | 1280000 | 239489 | 385888 |
| Anti-BTN3A moiety, scFab | | | | | | |
| fragment (n = 3) | | | | | | |
| bsAb031 | 0.046 | 1.400 | 661500 | 1006000 | 217693 | 336550 |
| bsAb032 | 0.203 | 0.846 | 418500 | 1150000 | 224314 | 428769 |
| bsAb068 | 0.049 | 0.787 | 681000 | 1049000 | 156112 | 312329 |
| Anti-BTN3A moiety, scFv | | | | | | |
| fragment (n = 13) | | | | | | |
| bsAb002 | 0.136 | 0.921 | 775000 | 1430000 | 235759 | 326438 |
| bsAb006 | 0.241 | 0.863 | 1215000 | 1900000 | 239517 | 294175 |
| bsAb009 | 0.515 | 0.921 | 411673 | 936000 | 197257 | 283190 |
| bsAb013 | 0.109 | 0.749 | 994500 | 1580000 | 206334 | 350298 |
| bsAb017 | 0.053 | 1.300 | 879500 | 1180000 | 221449 | 250258 |
| bsAb021 | 0.068 | 1.500 | 816000 | 944000 | 185864 | 275471 |
| bsAb034 | 0.167 | 1.900 | 706000 | 1245000 | 210433 | 380545 |
| bsAb037 | 0.086 | 1.100 | 585000 | 1250000 | 238482 | 269941 |
| bsAb039 | 0.187 | 2.100 | 1140000 | 1810000 | 294169 | 283471 |
| bsAb042 | 0.093 | ND | 824000 | 1240000 | 211567 | 361730 |
| bsAb053 | 0.393 | 0.984 | 494500 | 1245000 | 187258 | 423605 |
| bsAb059 | 0.056 | 1.200 | 815000 | 1495000 | 220740 | 458500 |
| bsAb060 | 0.166 | 0.899 | 961500 | 1245000 | 243466 | 390227 |

Anti-BTN3A × PD-L1 constructs (n = 35)

| | | | | | | |
|---|---|---|---|---|---|---|
| Anti-BTN3A moiety, Fab | | | | | | |
| fragment (n = 19) | | | | | | |
| bsAb003 | 0.073 | 0.030 | 500000 | 950000 | 722000 | 736000 |
| bsAb007 | 0.075 | 0.159 | 583500 | 1079500 | 761000 | 809500 |
| bsAb011 | 0.504 | 0.043 | 512500 | 1138000 | 681500 | 722000 |
| bsAb015 | 0.186 | 0.114 | 486500 | 1028500 | 706500 | 738500 |
| bsAb019 | 0.114 | 0.085 | 444500 | 1099500 | 795000 | 836500 |
| bsAb023 | 0.132 | 0.058 | 458500 | 1055500 | 804000 | 838000 |
| bsAb027 | 0.110 | ND | 215917 | 824000 | 220629 | 939000 |
| bsAb028 | 0.151 | ND | 546500 | 1196500 | 140995 | 614000 |
| bsAb035 | 0.068 | 0.097 | 525000 | 1028000 | 973000 | 1285000 |
| bsAb040 | 0.065 | 0.066 | 633500 | 1230000 | 1045000 | 1250000 |
| bsAb044 | 0.154 | ND | 584500 | 1012000 | 563000 | 953000 |
| bsAb046 | 0.065 | ND | 578500 | 1141500 | 678500 | 958500 |
| bsAb048 | 0.080 | ND | 592000 | 1015000 | 1180000 | 1365000 |
| bsAb050 | 0.080 | ND | 535000 | 1205000 | 1175000 | 1325000 |
| bsAb051 | 0.095 | 0.033 | 340140 | 792000 | 578500 | 597000 |
| bsAb055 | 0.076 | 0.108 | 344251 | 862000 | 820500 | 984500 |
| bsAb063 | 0.104 | 0.024 | 399639 | 981000 | 775000 | 810500 |
| bsAb064 | 0.225 | 0.025 | 383728 | 1086500 | 776500 | 800500 |
| bsAb065 | 0.145 | 0.034 | 290393 | 912500 | 887500 | 951500 |
| Anti-BTN3A moiety, scFab | | | | | | |
| (n = 3) | | | | | | |
| bsAb029 | 0.068 | 0.033 | 684500 | 1297000 | 796000 | 838500 |
| bsAb030 | 0.222 | 0.030 | 435000 | 1279000 | 897500 | 934500 |
| bsAb066 | 0.058 | 0.075 | 732500 | 1245000 | 821500 | 876500 |
| Anti-BTN3A moiety, scFv | | | | | | |
| (n = 13) | | | | | | |
| bsAb004 | 0.132 | 0.033 | 779500 | 1455000 | 716500 | 729000 |
| bsAb008 | 0.266 | 0.049 | 1150000 | 1820000 | 735500 | 786000 |
| bsAb012 | 0.537 | 0.035 | 376933 | 1083000 | 770000 | 799000 |
| bsAb016 | 0.122 | 0.037 | 1080000 | 1530000 | 769500 | 791000 |
| bsAb020 | 0.114 | 0.073 | 997000 | 1500000 | 746500 | 784500 |
| bsAb024 | 0.073 | 0.065 | 1031500 | 1665000 | 762000 | 794500 |
| bsAb036 | 0.457 | 0.055 | 682500 | 1223500 | 1030000 | 1235000 |
| bsAb041 | 0.101 | 0.053 | 1105000 | 1670000 | 1135000 | 1305000 |
| bsAb054 | 0.126 | 0.033 | 423500 | 1056500 | 663500 | 689500 |
| bsAb057 | 0.056 | 0.038 | 831000 | 1118000 | 685500 | 732500 |
| bsAb058 | 0.057 | 0.042 | 882000 | 1241000 | 726500 | 755000 |
| bsAb069 | 0.100 | 0.076 | 635000 | 1185000 | 1015000 | 1165000 |
| bsAb070 | 0.074 | 0.079 | 790500 | 1003000 | 1050000 | 1195000 |

Note:
rh (recombinant human);
NA (not applicable);
ND (not determined)

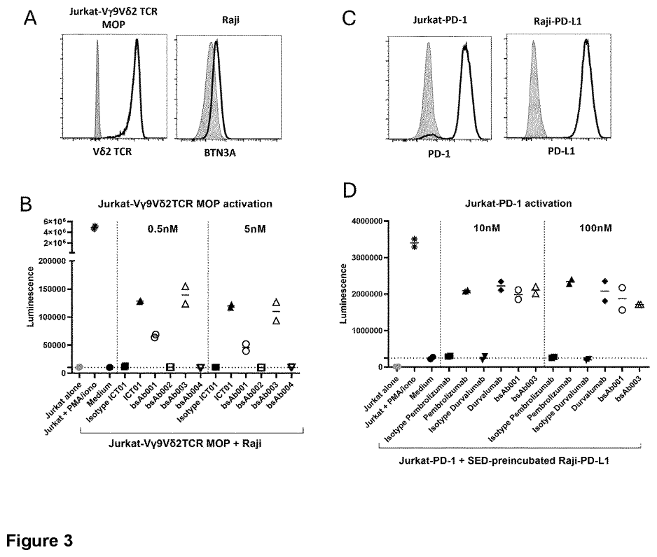

We proceeded to investigate the efficacy of these constructs in modulating Vγ9Vδ2 and γβ T cell responses, as previously reported for anti-BTN3A and anti-PD-1/PD-L1, respectively. Two cell-based reporter assays, involving Jurkat-Vγ9Vδ2 TCR MOP and Jurkat-PD-1 reporter assays, were conducted to assess the activity of anti-BTN3A and anti-PD-1/PD-L1 moieties, respectively. The parental mAbs, namely ICT01, Pembrolizumab, and Durvalumab, served as positive controls for anti-BTN3A, anti-PD-1 and anti-PD-L1 activities, respectively (FIG. 3).

To evaluate BTN3A agonist activity, Jurkat-Vγ9Vδ2 TCR MOP cells were co-cultured with Raji cells expressing BTN3A (FIG. 3A), and the activation of Jurkat-Vγ9Vδ2 TCR MOP cells was determined by luminescence. As expected, ICT01 induced activation of Jurkat-Vγ9Vδ2 TCR MOP cells when co-cultured with Raji cells, as evidenced by an increase in luminescence signal compared to controls (medium or corresponding isotype) (FIG. 3B). Constructs comprising an anti-BTN3A moiety in a Fab format were capable of inducing activation of Jurkat-Vγ9Vδ2 TCR MOP cells (Table 4).

For assessment of PD-1/PD-L1 blocking activity, 29 bsAbs retaining BTN3A agonist activity were further evaluated for their ability to enhance Jurkat-PD-1 cell activation in co-culture with Raji-PD-L1 cells pre-incubated with superantigen (SED). Co-culture with SED-preincubated Raji cells induced Jurkat-PD-1 cell activation which was markedly enhanced by a blocking anti-PD-1 mAb (Pembrolizumab) or anti-PD-L1 mAb (Durvalumab) as expected (FIG. 3D). 28 bsAbs were able to enhance Jurkat-PD-1 activation compared to the medium control at the two concentrations tested (Table 4). Finally, 23 out of 28 products with the higher purity were retained for further characterization (Table 4).

TABLE 4
Activity of bsAbs on Jurkat-Vy9Vo2 TCR MOP and Jurkat-PD-1 cells
(expressed as a fold-change to the medium condition)

| | Jurkat-Vy9Vo2 | Jurkat- Vy9V02 | | |
|---|---|---|---|---|
| | TCR MOP | TCR MOP | Jurkat-PD-1 | Jurkat-PD-1 |
| | activation at | activation at | activation at | activation at |
| bsAbs and Controls | 5 nM | 0.5 nM | 100 nM | 10 nM |
| Medium | 1.0 | 1.0 | 1.0 | 1.0 |
| Isotype Ctrl | | | | |
| Isotype ICT01 | 1.0 | 1.2 | NA | NA |
| Iso Durvalumab | NA | NA | 0.8 | 1.0 |
| Iso Pembrolizumab | NA | NA | 1.1 | 1.2 |
| Positive Ctrl | | | | |
| ICT01 | 11.7 | 12.5 | NA | NA |
| Pembrolizumab | NA | NA | 9.5 | 8.5 |
| Durvalumab | NA | NA | 8.4 | 9.0 |
| Anti-BTN3A moiety, Fab | | | | |
| (n = 18) | | | | |
| bsAb001 | 4.5 | 6.5 | 7.6 | 8.0 |
| bsAb005 | 4.2 | 6.0 | 6.9 | 7.3 |
| bsAb010 | 4.8 | 5.3 | 0.6 | 6.1 |
| bsAb014 | 4.6 | 6.4 | 1.9 | 5.9 |
| bsAb018 | 1.7 | 1.1 | NA | NA |
| bsAb022 | 1.4 | 1.3 | NA | NA |
| bsAb025* | 1.2 | 1.0 | NA | NA |
| bsAb026* | 1.4 | 2.9 | NA | NA |
| bsAb033 | 7.1 | 7.8 | 6.0 | 1.6 |
| bsAb038 | 7.2 | 8.5 | 3.5 | 1.5 |
| bsAb043* | 6.8 | 6.2 | 5.5 | 1.6 |
| bsAb045* | 6.3 | 4.3 | 5.8 | 1.7 |
| bsAb047 | 5.8 | 4.0 | 8.3 | 3.1 |
| bsAb049 | 6.2 | 5.9 | 2.3 | 1.3 |
| bsAb052* | 4.5 | 4.2 | 7.6 | 5.3 |
| bsAb056 | 10.0 | 7.0 | 3.4 | 1.5 |
| bsAb061 | 5.4 | 3.7 | 7.3 | 8.6 |
| bsAb062 | 5.8 | 4.3 | 6.8 | 7.8 |
| Anti-BTN3A moiety, | | | | |
| scFab (n = 3) | | | | |
| bsAb031 | 2.2 | 1.8 | NA | NA |
| bsAb032 | 1.0 | 0.9 | NA | NA |
| bsAb068 | 1.6 | 1.4 | NA | NA |
| Anti-BTN3A moiety, scFv | | | | |
| (n = 13) | | | | |
| bsAb002 | 1.0 | 1.0 | NA | NA |
| bsAb006 | 1.0 | 1.0 | NA | NA |
| bsAb009 | 0.8 | 1.0 | NA | NA |
| bsAb013 | 0.9 | 1.0 | NA | NA |
| bsAb017 | 1.5 | 1.3 | NA | NA |
| bsAb021 | 1.5 | 1.6 | NA | NA |
| bsAb034 | 1.1 | 0.9 | NA | NA |
| bsAb037 | 1.7 | 1.2 | NA | NA |
| bsAb039 | 1.0 | 0.9 | NA | NA |
| bsAb042 | 1.0 | 1.1 | NA | NA |
| bsAb053 | 1.1 | 1.2 | NA | NA |
| bsAb059 | 1.1 | 1.1 | NA | NA |
| bsAb060 | 1.1 | 1.1 | NA | NA |
| Anti-BTN3A moiety, Fab | | | | |
| (n = 19) | | | | |
| bsAb003 | 10.7 | 13.6 | 6.9 | 8.5 |
| bsAb007 | 10.0 | 11.8 | 7.5 | 8.8 |
| bsAb011 | 9.8 | 12.2 | 2.3 | 6.4 |
| bsAb015 | 7.3 | 8.2 | 7.8 | 8.9 |
| bsAb019 | 6.1 | 1.6 | 7.6 | 8.8 |
| bsAb023 | 5.2 | 2.2 | 8.6 | 7.4 |
| bsAb027 | 1.1 | 1.0 | NA | NA |
| bsAb028 | 1.2 | 1.6 | NA | NA |
| bsAb035 | 11.2 | 14.1 | 7.9 | 5.0 |
| bsAb040 | 8.8 | 10.4 | 7.5 | 6.0 |
| bsAb044* | 9.4 | 11.9 | 7.6 | 3.0 |
| bsAb046* | 8.9 | 8.6 | 7.5 | 3.8 |
| bsAb048 | 7.9 | 8.2 | 8.9 | 7.4 |
| bsAb050 | 9.3 | 10.1 | 7.8 | 6.9 |
| bsAb051 | 2.3 | 1.5 | NA | NA |
| bsAb055 | 11.8 | 2.9 | 7.4 | 5.8 |
| bsAb063 | 9.2 | 9.7 | 7.0 | 9.2 |
| bsAb064 | 9.2 | 8.8 | 6.8 | 6.7 |
| bsAb065 | 2.6 | 2.0 | | |
| Anti-BTN3A moiety, | | | | |
| scFab (n = 3) | | | | |
| bsAb029 | 1.6 | 1.2 | NA | NA |
| bsAb030 | 0.9 | 0.9 | NA | NA |
| bsAb066 | 2.1 | 2.0 | NA | NA |
| Anti-BTN3A moiety, scFv | | | | |
| (n = 13) | | | | |
| bsAb004 | 1.0 | 0.9 | NA | NA |
| bsAb008 | 1.0 | 0.9 | NA | NA |
| bsAb012 | 0.9 | 1.0 | NA | NA |
| bsAb016 | 0.8 | 1.1 | NA | NA |
| bsAb020 | 1.0 | 1.1 | NA | NA |
| bsAb024 | 1.0 | 1.0 | NA | NA |
| bsAb036 | 0.9 | 0.9 | NA | NA |
| bsAb041 | 1.0 | 0.9 | NA | NA |
| bsAb054 | 1.0 | 2.8 | NA | NA |
| bsAb057 | 1.1 | 1.2 | NA | NA |
| bsAb058 | 1.1 | 1.1 | NA | NA |
| bsAb069 | 3.8 | 2.4 | NA | NA |
| bsAb070 | 1.3 | 1.5 | NA | NA |

Note:
NA (Non applicable);
*(bsAbs with low purity)

### Example 4: Most bsAbs Demonstrate Greater BTN3A Agonist Activity Compared to ICT01

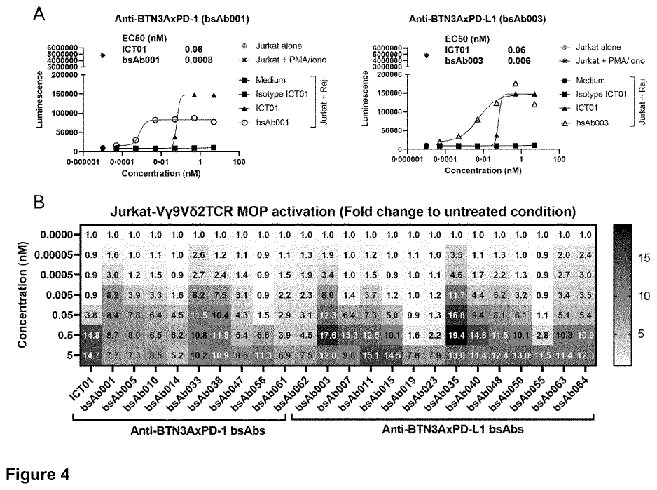

To assess the ability of the 23 selected bsAbs to induce Vγ9Vδ2 T cell activation in comparison to the parental mAb ICT01, we conducted dose-response analyses using both Jurkat-Vγ9Vδ2 TCR MOP cell line and primary Vγ9Vδ2 T cells. In the Jurkat-Vγ9Vδ2 TCR MOP reporter assay, all bsAbs induced dose-dependent activation of these reporter cells co-cultured with Raji cells, akin to ICT01 (FIG. 4). ICT01-induced Jurkat-Vγ9Vδ2 TCR MOP activation was evident at 0.05 nM whereas 18 out of the 23 bsAbs elicited Jurkat-Vγ9Vδ2 TCR MOP cell activation at a 10-fold lower dose (0.005 nM). Some constructs including bsAb001 and bsAb033 among anti-BTN3AxPD-1 constructs and bsAb003 and bsAb035 among anti-BTN3AxPD-L1 constructs, induced Jurkat-Vγ9Vδ2 TCR MOP cell activation at even lower doses, such as 0.0005 nM (refer to FIG. 4).

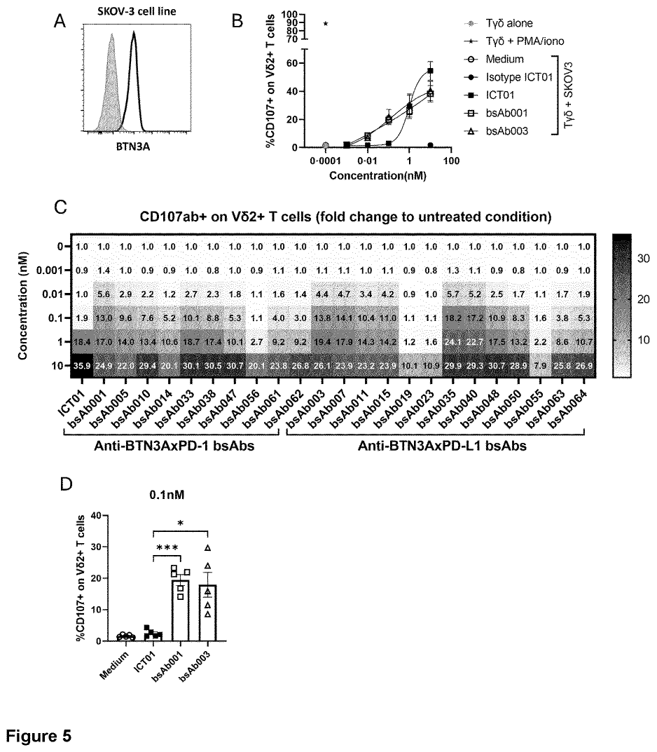

Consistent results were observed in the primary Vγ9Vδ2 T cell degranulation assay when expanded primary Vγ9Vδ2 T cells were co-cultured with an ovarian cancer cell line (SKOV3) which expresses BTN3A at the plasma membrane (FIG. 5A). 18 out of the 23 bsAbs induced Vγ9Vδ2T cell degranulation at 0.1 nM, with a mean increase in CD107ab expression on Vγ9Vδ2T cells ranging from 3- to 18.2-fold, depending on the construct, while ICT01 induced a 1.9-fold increase in CD107ab expression on Vγ9Vδ2T cells (FIGS. 5B and C).

Overall, our results confirm the ability of all selected bsAbs to enhance Vγ9Vδ2 T cell activation and highlight the superior potency of most constructs compared to the parental mAb ICT01.

### Example 5: Anti-BTN3AxPD-L1 bsAbs Demonstrate Greater Potency than ICT01 in Enhancing Vγ9Vδ2T Cell-Mediated Killing of Tumor Cells

Considering the strong effect of bsAbs on mediating Vγ9Vδ2 T cell activation, we investigated the activity of the bsAbs on tumor cell killing by culturing SKOV3 target tumor cells with primary Vγ9Vδ2 T cells expanded in vitro.

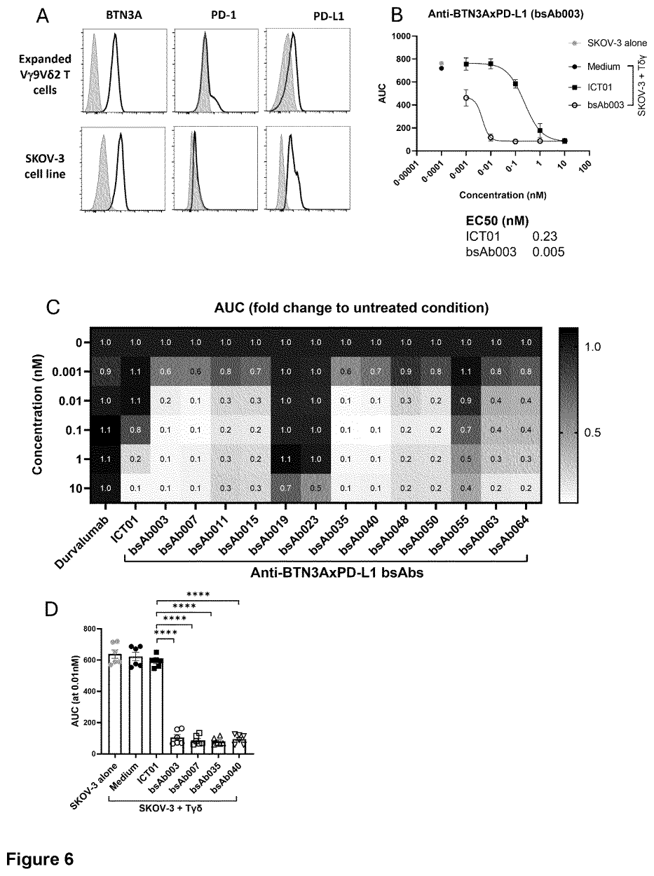

Initially, we assessed the expression of BTN3A and PD-1/PD-L1 on both tumor cells and expanded Vγ9Vδ2 T cells. As shown in FIG. 6A, BTN3A is prominently expressed at the surface of both Vγ9Vδ2 T cells and tumor cells (FIG. 6A, left). PD-L1 is highly expressed on SKOV3 cells, while PD-1 is detectable on Vγ9Vδ2 T cells but not on tumor cells, with an heterogeneous profile among donors (FIG. 6A, middle and right).

Subsequently, we evaluated the killing-inducing ability of anti-BTN3AxPD-L1 bsAbs compared to parental mAbs (ICT01 and Durvalumab) by monitoring the growth of cancer cells in co-culture with expanded Vγ9Vδ2 T cells over time using the Incucyte® live-imaging system (FIG. 6B-D). ICT01 treatment induced a dose-dependent killing of tumor cells by Vγ9Vδ2 T cells as shown by a significant decrease in target cell counts over time compared to the untreated condition, detectable from 0.1 nM ICT01 (FIGS. 6B and C). By contrast, treatment with Durvalumab had no effect (FIG. 6C). 10 out of the 13 anti-BTN3AxPD-L1 constructs tested including bsAb003, bsAb007, bsAb011, bsAb015, bsAb035, bsAb040, bsAb048, bsAb050, bsAb063 and bsAb064, showed superior potency to ICT01 in tumor cell killing as shown by a significant difference in elimination of SKOV3 cells even at the lowest doses tested, such as 0.001 and 0.01 nM (FIGS. 6C and D).

In summary, most selected anti-BTN3AxPD-1 and anti-BTN3AxPD-L1 bsAbs demonstrate significantly superior effects in modulating Vγ9Vδ2 T cell activation compared to ICT01. Ten constructs were found significantly more potent than ICT01 for tumor cell elimination in vitro.

### Example 6: Anti-BTN3AxPD-L1 bsAbs, with the scFv of Anti-PD-L1 Fused to the ICT01 N-Terminus of Either the Heavy Chain or the Light Chain, Exhibit Different Levels of Activity

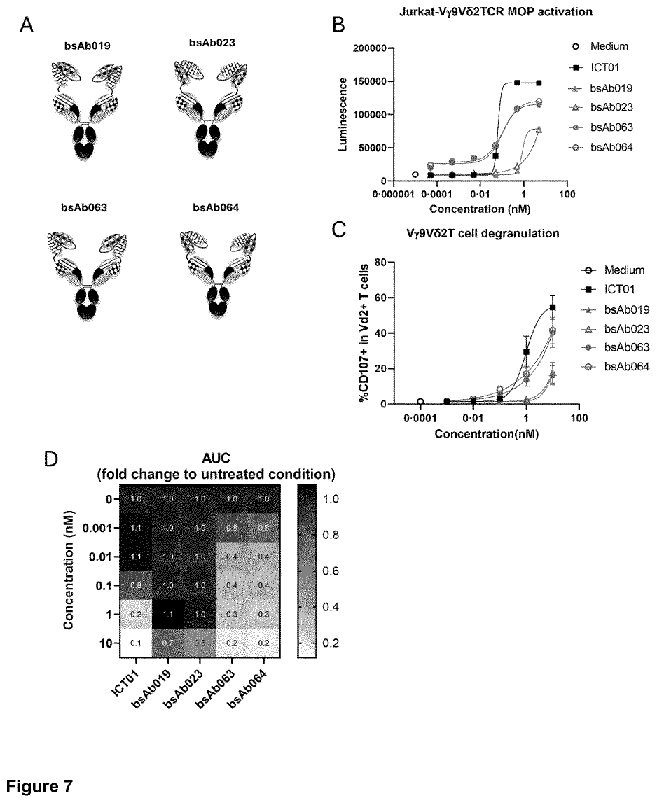

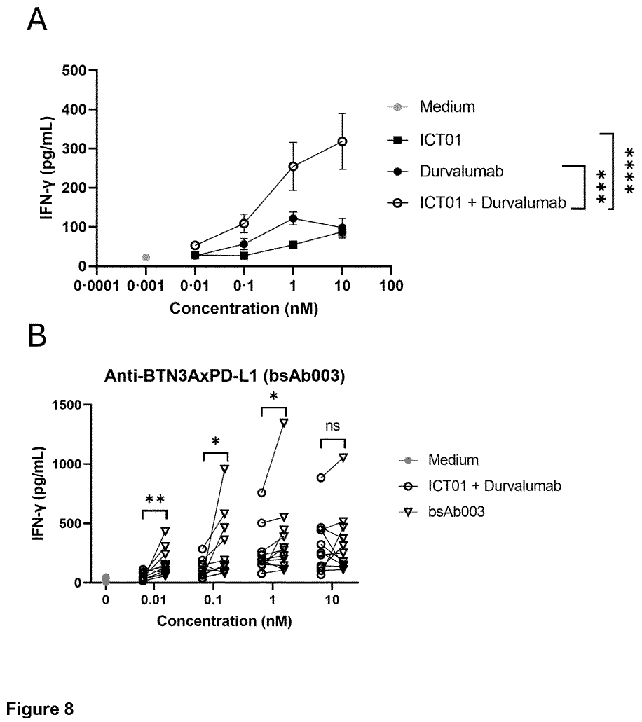

Subsequently, we evaluated the impact of bsAb design on the anti-BTN3AxPD-L1 bsAbs-mediated Vγ9Vδ2 T cell activity. We focused on four anti-BTN3AxPD-L1 bsAbs in which the scFv of anti PD-L1 is fused to the ICT01 N-terminus of the heavy chain (bsAb019 and bsAb023) or the light chain (bsAb063 and bsAb064) (FIG. 7A). Interestingly, the two anti-BTN3AxPD-L1 constructs comprising the anti-PD-L1 fused to the ICT01 N-terminus of the light chain (bsAb063 and bsAb064) were more effective than those fused to the ICT01 N-terminus of the heavy chain (bsAb019 and bsAb023) in mediating the activation of Jurkat-Vγ9Vδ2 TCR MOP cells (FIG. 7B) or primary expanded Vγ9Vδ2 T cells (FIG. 7C). Additionally, bsAb063 and bsAb064 showed superior potency and efficacy compared to bsAb019 and bsAb023 in tumor cell killing by expanded Vγ9Vδ2 T cells (FIG. 8D). These data indicate an advantage of anti-BTN3AxPD-L1 bsAbs comprising the anti PD-L1 fused to the ICT01 N-terminus of the light chain.

### Example 7: Anti-BTN3AxPD-L1 bsAbs Demonstrate Superior Activity in Mediating IFN-γ Secretion in a Mixed Lymphocyte Reaction (MLR) Assay than the Combination ICT01+Durvalumab

We conducted a comparative analysis between the reference bsAb003 (anti-BTN3AxPD-L1 bsAb), used as a reference bispecific antibody, and the combination of ICT01 with Durvalumab, for its ability to stimulate IFN-γ secretion of T cells by allogeneic monocyte-derived dendritic cells (moDC) in a MLR assay over 3 days.

First, we evaluated the efficacy of combining ICT01 with Durvalumab compared to each individual agent. As expected, Durvalumab led to a dose-dependent secretion of IFN-γ by T cells. A similar effect was observed with ICT01 treatment, albeit less pronounced. Remarkably, the combination of ICT01 with Durvalumab led to a significantly superior effect compared to the individual agents (FIG. 8A).

Subsequently, we examined the dose-dependent effect of the reference bsAb003 in comparison to the combination of ICT01 with Durvalumab. Treatment with bsAb003 elicited a dose-dependent production of IFN-γ by T cells (FIG. 8B). Notably, the IFN-γ secretion mediated by bsAbs003 was substantially greater compared to the combination of ICT01 and Durvalumab, particularly at lower doses (FIG. 8B). These findings suggest a significant advantage of the bsAbs in mediating responses of Vγ9Vδ2 and γβ T cells compared to the combination of ICT01 with Durvalumab.

### BIBLIOGRAPHICAL REFERENCES

Arnett, Heather A., et Joanne L. Viney. 2014. «Immune Modulation by Butyrophilins». Nature Reviews Immunology 14 (8): 559-69. https://doi.org/10.1038/nri3715.
Blazquez, Juan-Luis, Audrey Benyamine, Christine Pasero, et Daniel Olive. 2018. «New Insights Into the Regulation of Γδ T Cells by BTN3A and Other BTN/BTNL in Tumor Immunity». Frontiers in Immunology 9 (juillet): 1601. https://doi.org/10.3389/fimmu.2018.01601.
Freeman, Gordon J., Andrew J. Long, Yoshiko Iwai, Karen Bourque, Tatyana Chernova, Hiroyuki Nishimura, Lori J. Fitz, et al. 2000. «Engagement of the Pd-1 Immunoinhibitory Receptor by a Novel B7 Family Member Leads to Negative Regulation of Lymphocyte Activation». Journal of Experimental Medicine 192 (7): 1027-34. https://doi.org/10.1084/jem. 192.7.1027.
Gentles, Andrew J., Aaron M. Newman, Chih Long Liu, Scott V. Bratman, Weiguo Feng, Dongkyoon Kim, Viswam S. Nair, et al. 2015. «The Prognostic Landscape of Genes and Infiltrating Immune Cells across Human Cancers». Nature Medicine 21 (8): 938-45. https://doi.org/10.1038/nm.3909.
Girard, Pauline, Julie Charles, Camille Cluzel, Emmanuelle Degeorges, Olivier Manches, Joel Plumas, Florence De Fraipont, et al. 2019. «The features of circulating and tumor-infiltrating γδ T cells in melanoma patients display critical perturbations with prognostic impact on clinical outcome». Oncoimmunology 8 (8): 1601483. https://doi.org/10.1080/2162402X.2019.1601483.
Guo, Qingming, Peng Zhao, Zhen Zhang, Jinyu Zhang, Zheng Zhang, Yanan Hua, Bin Han, Ning Li, Xiaowen Zhao, et Lin Hou. 2020. «TIM-3 Blockade Combined with Bispecific Antibody MT110 Enhances the Anti-Tumor Effect of Γδ T Cells». Cancer Immunology, Immunotherapy 69 (12): 2571-87. https://doi.org/10.1007/s00262-020-02638-0.
Hirano, Fumiya, Katsumi Kaneko, Hideto Tamura, Haidong Dong, Shengdian Wang, Masao Ichikawa, Cecilia Rietz, et al. 2005. «Blockade of B7-H1 and PD-1 by Monoclonal Antibodies Potentiates Cancer Therapeutic Immunity». Cancer Research 65 (3): 1089-96.
Hoeres, Timm, Elisabeth Holzmann, Manfred Smetak, Josef Birkmann, et Martin Wilhelm. 2019. «PD-1 signaling modulates interferon-γ production by Gamma Delta (γδ) T-Cells in response to leukemia». Oncoimmunology 8 (3): 1550618. https://doi.org/10.1080/2162402X.2018.1550618.
Holtmeier, Wolfgang, et Dieter Kabelitz. 2005. «Γδ T Cells Link Innate and Adaptive Immune Responses». Mechanisms of Epithelial Defense 86:151-83. https://doi.org/10.1159/000086659.
Iwai, Yoshiko, Masayoshi Ishida, Yoshimasa Tanaka, Taku Okazaki, Tasuku Honjo, et Nagahiro Minato. 2002. «Involvement of PD-L1 on tumor cells in the escape from host immune system and tumor immunotherapy by PD-L1 blockade». Proceedings of the National Academy of Sciences 99 (19): 12293-97. https://doi.org/10.1073/pnas. 192461099.
Iwai, Yoshiko, Seigo Terawaki, et Tasuku Honjo. 2005. «PD-1 blockade inhibits hematogenous spread of poorly immunogenic tumor cells by enhanced recruitment of effector T cells». International Immunology 17 (2): 133-44. https://doi.org/10.1093/intimm/dxh194.
Jiang, Youhai, Xiaofang Zhao, Jing Fu, et Hongyang Wang. 2020. «Progress and Challenges in Precise Treatment of Tumors With PD-1/PD-L1 Blockade». Frontiers in Immunology 11:339. https://doi.org/10.3389/fimmu.2020.00339.
Jin, Zhenyi, Tianbi Lan, Yun Zhao, Jinxia Du, Jie Chen, Jing Lai, Ling Xu, et al. 2022. «Higher TIGIT+CD226− γδ T cells in Patients with Acute Myeloid Leukemia». Immunological Investigations 51 (1): 40-50. https://doi.org/10.1080/08820139.2020.1806868.
Kabelitz, Dieter, Ruben Serrano, Léonce Kouakanou, Christian Peters, et Shirin Kalyan. 2020. «Cancer Immunotherapy with Γδ T Cells: Many Paths Ahead of Us». Cellular & Molecular Immunology 17 (9): 925-39. https://doi.org/10.1038/s41423-020-0504-x.
Li, Xiaomi, Huimin Lu, Yanzheng Gu, Xueguang Zhang, Guangbo Zhang, Tongguo Shi, et Weichang Chen. 2020. «Tim-3 Suppresses the Killing Effect of Vγ9Vδ2 T Cells on Colon Cancer Cells by Reducing Perforin and Granzyme B Expression». Experimental Cell Research 386 (1): 111719. https://doi.org/10.1016/j.yexcr.2019.111719.
McDermott D. F. and Atkins M. B. 2013 PD-1 as a potential target in cancer therapy. Cancer Medicine. 2 (5): 662-673.
Nishimura, Hiroyuki, Masato Nose, Hiroshi Hiai, Nagahiro Minato, et Tasuku Honjo. 1999. «Development of Lupus-like Autoimmune Diseases by Disruption of the PD-1 Gene Encoding an ITIM Motif-Carrying Immunoreceptor». Immunity 11 (2): 141-51. https://doi.org/10.1016/S1074-7613 (00) 80089-8.
Pauza, C. David, Mei-Ling Liou, Tyler Lahusen, Lingzhi Xiao, Rena G. Lapidus, Cristiana Cairo, et Haishan Li. 2018. «Gamma Delta T Cell Therapy for Cancer: It Is Good to be Local». Frontiers in Immunology 9. https://www.frontiersin.org/article/10.3389/fimmu.2018.01305.
Payne, Kyle K., Jessica A. Mine, Subir Biswas, Ricardo A. Chaurio, Alfredo Perales-Puchalt, Carmen M. Anadon, Tara Lee Costich, et al. 2020. «BTN3A1 governs anti-tumor responses by coordinating alpha-beta and gamma-delta T cells». Science (New York, N.Y.) 369 (6506): 942-49. https://doi.org/10.1126/science.aay2767.
Rhodes, David A., Walter Reith, et John Trowsdale. 2016. «Regulation of Immunity by Butyrophilins». Annual Review of Immunology 34 (1): 151-72. https://doi.org/10.1146/annurev-immunol-041015-055435.
Ribas, Antoni, et Jedd D. Wolchok. 2018. «Cancer immunotherapy using checkpoint blockade». Science 359 (6382): 1350-55. https://doi.org/10.1126/science.aar4060.
Robert, Caroline, Jacob Schachter, Georgina V. Long, Ana Arance, Jean Jacques Grob, Laurent Mortier, Adil Daud, et al. 2015. «Pembrolizumab versus Ipilimumab in Advanced Melanoma». New England Journal of Medicine 372 (26): 2521-32. https://doi.org/10.1056/NEJMoa1503093.
Stech, M., O. Nikolaeva, L. Thoring, W. F. M. Stöcklein, D. A. Wüstenhagen, M. Hust, S. Dübel, et S. Kubick. 2017. «Cell-Free Synthesis of Functional Antibodies Using a Coupled in Vitro Transcription-Translation System Based on CHO Cell Lysates». Scientific Reports 7 (1): 12030. https://doi.org/10.1038/s41598-017-12364-w.
Strome, Scott E., Haidong Dong, Hideto Tamura, Stephen G. Voss, Dallas B. Flies, Koji Tamada, Diva Salomao, et al. 2003. «B7-H1 Blockade Augments Adoptive T-Cell Immunotherapy for Squamous Cell Carcinoma». Cancer Research 63 (19): 6501-5.
Tosolini, Marie, Frédéric Pont, Mary Poupot, François Vergez, Marie-Laure Nicolau-Travers, David Vermijlen, Jean-Emmanuel Sarry, Francesco Dieli, et Jean-Jacques Fournié. 2017. «Assessment of tumor-infiltrating TCRVγ9Vδ2 γδ lymphocyte abundance by deconvolution of human cancers microarrays». Oncolmmunology 6 (3): e1284723. https://doi.org/10.1080/2162402X.2017.1284723.
Uomo, Generoso. 2012. «Lesson from Acute Experimental Pancreatitis: Multidrug Strategies Is Effective than Single-Target Therapy». JOP. Journal of the Pancreas 13 (5): 543-44. https://doi.org/10.6092/1590-8577/1210.
Wei, Spencer C., Colm R. Duffy, et James P. Allison. 2018. «Fundamental Mechanisms of Immune Checkpoint Blockade Therapy». Cancer Discovery 8 (9): 1069-86. https://doi.org/10.1158/2159-8290.CD-18-0367.
Wu, Kangni, Juan Feng, Yanghui Xiu, Zhifeng Li, Zhijuan Lin, Haijun Zhao, Hanyan Zeng, Weilin Xia, Lian Yu, et Bing Xu. 2020. «Vδ2 T Cell Subsets, Defined by PD-1 and TIM-3 Expression, Present Varied Cytokine Responses in Acute Myeloid Leukemia Patients». International Immunopharmacology 80 (mars): 106122. https://doi.org/10.1016/j.intimp.2019.106122.
Wu, Kangni, Haijun Zhao, Yanghui Xiu, Zhifeng Li, Jintao Zhao, Shiting Xie, Hanyan Zeng, Haiping Zhang, Lian Yu, et Bing Xu. 2019. «IL-21-Mediated Expansion of Vγ9Vδ2 T Cells Is Limited by the Tim-3 Pathway». International Immunopharmacology 69 (avril): 136-42. https://doi.org/10.1016/j.intimp.2019.01.027.
Xiang, Zheng, et Wenweil Tu. 2017. «Dual Face of Vγ9Vδ2-T Cells in Tumor Immunology: Anti-versus Activities». Frontiers in Immunology 8 Pro-Tumoral https://www.frontiersin.org/articles/10.3389/fimmu.2017.01041.

1. A bispecific antibody comprising at least
(i) a first antigen-binding moiety selected from:
anti-BTN3A antibodies or antigen-binding fragments thereof which comprise (a) a variable heavy chain (VH) polypeptide comprising an amino acid sequence that is at least about 95% identical to the amino acid sequence of SEQ ID NO: 1, and (b) a variable light chain (VL) polypeptide comprising an amino acid sequence that is at least about 95% identical to SEQ ID NO: 2,
anti-BTN3A antibodies or antigen-binding fragments thereof which comprise (a) a variable heavy chain (VH) polypeptide comprising an amino acid sequence that is at least about 95% identical to the amino acid sequence of SEQ ID NO: 48, and (b) a variable light chain (VL) polypeptide comprising an amino acid sequence that is at least about 95% identical to SEQ ID NO: 49;
anti-BTN3A antibodies or antigen-binding fragments thereof which comprise HCDRs1-3 of SEQ ID NO:3-5 and LCDRs1-3 of SEQ ID NO:6-8, and
anti-BTN3A antibodies or antigen-binding fragments thereof which comprise HCDR1 of SEQ ID NO:50, HCDR2 of SEQ ID NO:51 or 56 to 59, HCDR3 of SEQ ID NO:52 and LCDR1 of SEQ ID NO: 53, 60 or 61, LCDR2 of SEQ ID NO: 54 and LCDR3 of SEQ ID NO: 55;
and
(ii) a second antigen-binding moiety selected from:
anti-PD-L1 antibodies or antigen-binding fragments thereof which comprise (a) a variable heavy chain (VH) polypeptide comprising an amino acid sequence that is at least about 95% identical to the amino acid sequence of SEQ ID NO: 17, and (b) a variable light chain (VL) polypeptide comprising an amino acid sequence that is at least about 95% identical to SEQ ID NO: 18;
anti-PD-L1 antibodies which comprise HCDRs1-3 of SEQ ID NO:19-21 and LCDRs1-3 of SEQ ID NO:22-24;
anti-PD-L1 antibodies selected from durvalumab, atezolizumab, avelumab, BMS-936559, LY3300054, pacmilimab, FAZ053, envafolimab, and MDX-1105;
anti-PD-1 antibodies which comprise (a) a variable heavy chain (VH) polypeptide comprising an amino acid sequence that is at least about 95% identical to the amino acid sequence of SEQ ID NO: 9, and (b) a variable light chain (VL) polypeptide comprising an amino acid sequence that is at least about 95% identical to SEQ ID NO: 10;
anti-PD-1 antibodies which comprise HCDRs1-3 of SEQ ID NO:11-13 and LCDRs 1-3 of SEQ ID NO:14-16, and
anti-PD-1 antibodies selected from pembrolizumab, nivolumab, dostarlimab, retifanlimab, spartalizumab, MEDI-0680, cemiplimab, pidilizumab, pucotenlimab, zimberelimab, prolgolimab, penpulimab, toripalimab, tislelizumab, camrelizumab, AM-0001, STI-1110, balstilimab, sintilimab and SG001, or an antigen-binding fragment thereof.

2. The bispecific antibody according to claim 1, wherein the first and/or second antigen-binding moieties are selected from an immunoglobulin molecule and an antigen-binding fragment selected from a Fab fragment, a Fab′ fragment, a F(ab′)2 fragment, a single domain antibody, a scFab fragment and a scFv fragment.

3. The bispecific antibody according to claim 1, wherein one of the first and second antigen-binding moieties is an IgG molecule and the other of the first and second antigen-binding moieties is an antigen-binding fragment linked to the IgG molecule, wherein the antigen-binding fragment is selected from a Fab fragment, a Fab′ fragment, a F(ab′)2 fragment, a single domain antibody, a scFab fragment and a scFv fragment.

4. The bispecific antibody according to claim 1, wherein the first antigen-binding moiety is an IgG molecule and the second antigen-binding moiety is an antigen-binding moiety selected from a Fab fragment, a Fab′ fragment, a F(ab′)2 fragment, a single domain antibody, a scFab fragment and a scFv fragment, .

5. The bispecific antibody according to claim 1, which comprises:
(i) two heavy chains having the formula [scFv-linker-IgG(H)] and two light chains having the formula [IgG(L)];
(ii) two heavy chains having the formula [IgG(H)-linker-scFv] and two light chains having the formula [IgG(L)];
(iii) two heavy chains having the formula [IgG(H)] and two light chains having the formula [scFv-linker-IgG(L)];
(iv) two heavy chains having the formula [IgG(H)] and two light chains having the formula [IgG(L)-linker-scFv];
(v) one heavy chain having the formula [scFv-linker-IgG(H)], one heavy chain having the formula [IgG(H)] and two light chains having the formula [IgG(L)];
(vi) one heavy chain having the formula [IgG(H)-linker-scFv], one heavy chain having the formula [IgG(H)] and two light chains having the formula [IgG(L)];
(vii) two heavy chains having the formula [IgG(H)], one light chain having the formula [scFv-linker-IgG(L)] and one light chain having the formula [IgG(L)];
(viii) two heavy chains having the formula [IgG(H)], one light chain having the formula [IgG(L)-linker-scFv] and one light chain having the formula [IgG(L)];
(ix) one heavy chain having the formula [IgG(H)], one heavy chain having the formula [scFv-linker-IgG Fc] and one light chain having the formula [IgG(L)];
(x) one heavy chain having the formula [IgG(H)-linker-scFv], one heavy chain having the formula [IgG Fc-linker-scFv] and one light chain having the formula [IgG(L)];
(xi) two heavy chains having the formula [scFab-linker-IgG(H)] and two light chains having the formula [IgG(L)];
(xii) two heavy chains having the formula [IgG(H)-linker-scFab] and two light chains having the formula [IgG(L)];
(xiii) two heavy chains having the formula [IgG(H)] and two light chains having the formula [IgG(L)-linker-scFab]; or
(xiv) two heavy chains having the formula [IgG(H)] and two light chains having the formula [scFab-linker-IgG(L)],
wherein IgG(H) and IgG(L) are, respectively, the heavy chain and light chain of the IgG molecule that specifically binds to BTN3A or PD-1 or PD-L1, wherein IgG Fc is an IgG Fc region, and wherein scFv or scFab is the fragment that specifically binds to PD-1 or PD-L1 or BTN3A, wherein, where more than one scFv or scFab is present, said scFvs or scFabs are identical or different and, where more than one linker is present, said linkers are identical or different.

6. The bispecific antibody of claim 5, wherein the linker comprises the amino acid sequence (GGGGS)p, wherein p is an integer comprised between 1 and 8, or wherein the linker comprises an amino acid sequence selected from the sequences defined in SEQ ID NOs: 29 to 42.

7. The bispecific antibody of claim 1, wherein the first and second-antigen binding moieties are derived from human, chimeric or humanized antibodies.

8. The bispecific antibody according to claim 1, wherein said bispecific antibody is capable of promoting the activation of Vγ9Vδ2 T cells, in the presence of PD-L1 expressing target cells.

9. (canceled)

10. The bispecific antibody according to claim 1, wherein said second antigen-binding moiety binds specifically to PD-L1.

11. (canceled)

12. The bispecific antibody according to claim 1, wherein said second antigen-binding moiety is selected from durvalumab, atezolizumab, avelumab, BMS-936559, LY3300054, pacmilimab, FAZ053, envafolimab, and MDX-1105, or an antigen-binding fragment thereof.

13. The bispecific antibody according to claim 10, which comprises a heavy chain comprising an amino acid sequence that is at least about 95% identical to the amino acid sequence of SEQ ID NO:64 and a light chain comprising an amino acid sequence that is at least about 95% identical to the amino acid sequence of SEQ ID NO:65.

14. (canceled)

15. (canceled)

16. (canceled)

17. (canceled)

18. The bispecific antibody according to claim 1 which comprises a heavy chain comprising an amino acid sequence that is at least about 95% identical to the amino acid sequence of SEQ ID NO:62 and a light chain comprising an amino acid sequence that is at least about 95% identical to the amino acid sequence of SEQ ID NO:63.

19. (canceled)

20. The bispecific antibody according to claim 1, wherein said bispecific antibody comprises an IgG molecule, wherein the IgG molecule comprises:
CH1, CH2 and CH3 domains comprising the amino acid sequence set forth in SEQ ID NO: 26 or an amino acid sequence at least 95% identical therewith, or
CH2 and CH3 domains comprising the amino acid sequence set forth in SEQ ID NO:27 or 28 or an amino acid sequence at least 95% identical therewith, or
a CL domain comprising the amino acid sequence set forth in SEQ ID NO:25 or an amino acid sequence at least 95% identical therewith.

21. A polynucleotide encoding a light chain and/or a heavy chain of the bispecific antibody of claim 1.

22. An expression vector, comprising the polynucleotide according to claim 21.

23. A prokaryotic or eukaryotic host cell comprising the polynucleotide according to claim 21.

24. A method of producing the bispecific antibody according to claim 1, comprising the steps of a) transforming a host cell with vectors comprising one or more polynucleotides encoding said bispecific antibody, b) culturing the host cell under conditions suitable for the expression of the bispecific antibody and c) recovering the bispecific antibody from the culture.

25. A pharmaceutical composition comprising the bispecific antibody of claim 1, and a pharmaceutically acceptable carrier.

26. A method for treating cancer in a subject in need therefore comprising administering a therapeutically effective amount of the bispecific antibody of claim 1 to said subject.

27. (canceled)

28. The method of claim 26, wherein said bispecific antibody is used in combination with one or more additional therapeutic agents.


SEQUENCE LISTING (74 sequences)

SEQ ID NO: 1 - protein, 122 aa, Homo sapiens [sequence omitted]

SEQ ID NO: 2 - protein, 107 aa, Homo sapiens
DIQMTQSPSSLSASVGDRVTITCHASQNINVWLSWYQQKPGKAPKLLIYKASNLHTGVPSRFTGSGSGTDFTFTISSLQPEDIATYYCQQGQTYPYTFGQGTKLEIK

SEQ ID NO: 3 - protein, 5 aa, Homo sapiens
RYYMY

SEQ ID NO: 4 - protein, 17 aa, Homo sapiens
EINPNNGGTKFNEKFKN

SEQ ID NO: 5 - protein, 13 aa, Homo sapiens
EDDYDGTPFAMDY

SEQ ID NO: 6 - protein, 11 aa, Homo sapiens
HASQNINVWLS

SEQ ID NO: 7 - protein, 7 aa, Homo sapiens
KASNLHT

SEQ ID NO: 8 - protein, 9 aa, Homo sapiens
QQGQTYPYT

SEQ ID NO: 9 - protein, 120 aa, Homo sapiens [sequence omitted]

SEQ ID NO: 10 - protein, 111 aa, Homo sapiens [sequence omitted]

SEQ ID NO: 11 - protein, 5 aa, Homo sapiens
NYYMY

SEQ ID NO: 12 - protein, 17 aa, Homo sapiens
GINPSNGGTNFNEKFKN

SEQ ID NO: 13 - protein, 11 aa, Homo sapiens
RDYRFDMGFDY

SEQ ID NO: 14 - protein, 15 aa, Homo sapiens
RASKGVSTSGYSYLH

SEQ ID NO: 15 - protein, 7 aa, Homo sapiens
LASYLES

SEQ ID NO: 16 - protein, 9 aa, Homo sapiens
QHSRDLPLT

SEQ ID NO: 17 - protein, 121 aa, Homo sapiens
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 18 - protein, 108 aa, Homo sapiens
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 19 - protein, 5 aa, Homo sapiens
RYWMS

SEQ ID NO: 20 - protein, 17 aa, Homo sapiens
NIKQDGSEKYYVDSVKG

SEQ ID NO: 21 - protein, 12 aa, Homo sapiens
EGGWFGELAFDY

SEQ ID NO: 22 - protein, 12 aa, Homo sapiens
RASQRVSSSYLA

SEQ ID NO: 23 - protein, 7 aa, Homo sapiens
DASSRAT

SEQ ID NO: 24 - protein, 9 aa, Homo sapiens
QQYGSLPWT

SEQ ID NO: 25 - protein, 107 aa, Homo sapiens [sequence omitted]

SEQ ID NO: 26 - protein, 330 aa, Homo sapiens
  source 1..330: note = X is a lysine (K) or is absent
  VARIANT 330: note = X is a lysine (K) or is absent
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 27 - protein, 217 aa, Homo sapiens
  source 1..217: note = X is a lysine (K) or is absent
  VARIANT 217: note = X is a lysine (K) or is absent
APEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGX

SEQ ID NO: 28 - protein, 217 aa, Homo sapiens
  VARIANT 217: note = X is a lysine (K) or is absent
APEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGX

SEQ ID NO: 29 - protein, 11 aa, synthetic construct
SGGGGSGGGGS

SEQ ID NO: 30 - protein, 13 aa, synthetic construct
SGGGGSGGGGSAP

SEQ ID NO: 31 - protein, 5 aa, synthetic construct
NFSQP

SEQ ID NO: 32 - protein, 5 aa, synthetic construct
KRTVA

SEQ ID NO: 33 - protein, 8 aa, synthetic construct
GGGSGGGG

SEQ ID NO: 34 - protein, 10 aa, synthetic construct
GGGGSGGGGS

SEQ ID NO: 35 - protein, 15 aa, synthetic construct
GGGGSGGGGSGGGGS

SEQ ID NO: 36 - protein, 15 aa, synthetic construct
THTCPPCPEPKSSDK

SEQ ID NO: 37 - protein, 4 aa, synthetic construct
GGGS

SEQ ID NO: 38 - protein, 13 aa, synthetic construct
EAAKEAAKGGGGS

SEQ ID NO: 39 - protein, 8 aa, synthetic construct
EAAKEAAK

SEQ ID NO: 40 - protein, 5 aa, synthetic construct
GGGGS

SEQ ID NO: 41 - protein, 50 aa, synthetic construct
GGSSGSGSGSTGTSSSGTGTSAGTTGTSASTSGSGSGGGGGSGGGGSAGG

SEQ ID NO: 42 - protein, 20 aa, synthetic construct
GGGGSGGGGSGGGGSGGGGS

SEQ ID NO: 43 - protein, 513 aa, Homo sapiens
MKMASFLAFLLLNFRVCLLLLQLLMPHSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAGTLPVLLLLLGGAGYFLWQQQEEKKTQFRKKKREQELREMAWSTMKQEQSTRVKLLEELRWRSIQYASRGERHSAYNEWKKALFKPADVILDPKTANPILLVSEDQRSVQRAKEPQDLPDNPERFNWHYCVLGCESFISGRHYWEVEVGDRKEWHIGVCSKNVQRKGWVKMTPENGFWTMGLTDGNKYRTLTEPRTNLKLPKPPKKVGVFLDYETGDISFYNAVDGSHIHTFLDVSFSEALYPVFRILTLEPTALTICPA

SEQ ID NO: 44 - protein, 334 aa, Homo sapiens
MKMASSLAFLLLNFHVSLLLVQLLTPCSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSNLHVEVKGYEDGGIHLECRSTGWYPQPQIQWSNAKGENIPAVEAPVVADGVGLYEVAASVIMRGGSGEGVSCIIRNSLLGLEKTASISIADPFFRSAQPWIAALAGTLPILLLLLAGASYFLWRQQKEITALSSEIESEQEMKEMGYAATEREISLRESLQEELKRKKIQYLTRGEESSSDTNKSA

SEQ ID NO: 45 - protein, 584 aa, Homo sapiens
MKMASSLAFLLLNFHVSLFLVQLLTPCSAQFSVLGPSGPILAMVGEDADLPCHLFPTMSAETMELRWVSSSLRQVVNVYADGKEVEDRQSAPYRGRTSILRDGITAGKAALRIHNVTASDSGKYLCYFQDGDFYEKALVELKVAALGSDLHIEVKGYEDGGIHLECRSTGWYPQPQIKWSDTKGENIPAVEAPVVADGVGLYAVAASVIMRGSSGGGVSCIIRNSLLGLEKTASISIADPFFRSAQPWIAALAGTLPISLLLLAGASYFLWRQQKEKIALSRETEREREMKEMGYAATEQEISLREKLQEELKWRKIQYMARGEKSLAYHEWKMALFKPADVILDPDTANAILLVSEDQRSVQRAEEPRDLPDNPERFEWRYCVLGCENFTSGRHYWEVEVGDRKEWHIGVCSKNVERKKGWVKMTPENGYWTMGLTDGNKYRALTEPRTNLKLPEPPRKVGIFLDYETGEISFYNATDGSHIYTFPHASFSEPLYPVFRILTLEPTALTICPIPKEVESSPDPDLVPDHSLETPLTPGLANESGEPQAEVTSLLLPAHPGAEVSPSATTNQNHKLQARTEALY

SEQ ID NO: 46 - DNA, 1356 bp, Homo sapiens
  source 1..1356: mol_type = other DNA
caggtccaactggtgcagtctggggctgaagtgaagaagcctggggcttcagtgaagttgtcctgcaaggcttctggctacatcttcaccagatactatatgtattgggtgaagcagaggcctggacaaggccttgagtggattggagagattaatcctaacaatggtggtactaagttcaatgagaagttcaagaacagggccacactgactgtagacaaatccatcagcacagcatacatggagctcagcaggctgagatctgacgacacggcggtctattattgttcaagagaggatgattacgacgggaccccctttgctatggactactggggtcaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaattcgaggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttga

SEQ ID NO: 47 - DNA, 645 bp, Homo sapiens
  source 1..645: mol_type = other DNA
gacatccagatgacccagtctccatccagtctgtctgcatccgtaggagacagagtcaccatcacttgccatgccagtcagaacattaatgtttggttatcttggtaccagcagaaaccaggaaaagcccctaaactcttgatctataaggcttccaacttgcacacaggcgtcccatcaagatttactggcagtggatctggaacagatttcacattcaccatcagcagcctgcagcctgaagacattgccacttactactgtcaacagggtcaaacttatccatacacgttcggacaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 48 - protein, 122 aa, Homo sapiens
QVQLVQSGAEVKKPGASVKVSCKASGYTFTRYYLYWVKQRPGQGLEWIGEINPNNGGTKFNEKFKSRATMTVDKSTRTTYMELSSLRSEDTAVYYCSREDDYDGTPDAMDYWGQGTLVTVSS

SEQ ID NO: 49 - protein, 107 aa, Homo sapiens
DIQMTQSPSSLSASVGDRVTITCHASQNINLWLSWYQQKPGKAPKLLIYRASNLHTGVPSRFSGSGSATDFTFTISSLQPEDIATYYCQQGHSYPYTFGGGTKVDIK

SEQ ID NO: 50 - protein, 5 aa, Homo sapiens
RYYLY

SEQ ID NO: 51 - protein, 17 aa, Homo sapiens
EINPNNGGTKFNEKFKS

SEQ ID NO: 52 - protein, 13 aa, Homo sapiens
EDDYDGTPDAMDY

SEQ ID NO: 53 - protein, 11 aa, Homo sapiens
HASQNINLWLS

SEQ ID NO: 54 - protein, 7 aa, Homo sapiens
RASNLHT

SEQ ID NO: 55 - protein, 9 aa, Homo sapiens
QQGHSYPYT

SEQ ID NO: 56 - protein, 17 aa, Homo sapiens
EINPSNGGTNFNEKFKS

SEQ ID NO: 57 - protein, 17 aa, Homo sapiens
EINPSNGGTNFAQKFQG

SEQ ID NO: 58 - protein, 17 aa, Homo sapiens
EINPSNGGTNFNEKLKS

SEQ ID NO: 59 - protein, 17 aa, Homo sapiens
EINPSNGGTNFNESLKS

SEQ ID NO: 60 - protein, 11 aa, Homo sapiens
HASQNINVWLS

SEQ ID NO: 61 - protein, 11 aa, Homo sapiens
RASQGISVWLS

SEQ ID NO: 62 - protein, 722 aa, Synthetic construct
QVQLVQSGAEVKKPGASVKLSCKASGYIFTRYYMYWVKQRPGQGLEWIGEINPNNGGTKFNEKFKNRATLTVDKSISTAYMELSRLRSDDTAVYYCSREDDYDGTPFAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIK

SEQ ID NO: 63 - protein, 214 aa, Synthetic construct
DIQMTQSPSSLSASVGDRVTITCHASQNINVWLSWYQQKPGKAPKLLIYKASNLHTGVPSRFTGSGSGTDFTFTISSLQPEDIATYYCQQGQTYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 720 aa, Synthetic construct
QVQLVQSGAEVKKPGASVKLSCKASGYIFTRYYMYWVKQRPGQGLEWIGEINPNNGGTKFNEKFKNRATLTVDKSISTAYMELSRLRSDDTAVYYCSREDDYDGTPFAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSGGGGSGGGGSGGGGSGGGGSEIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 65 - protein, 214 aa, Synthetic construct; identical to SEQ ID NO: 63

SEQ ID NO: 66 - protein, 249 aa, Synthetic construct
QVQLVQSGAEVKKPGASVKLSCKASGYIFTRYYMYWVKQRPGQGLEWIGEINPNNGGTKFNEKFKNRATLTVDKSISTAYMELSRLRSDDTAVYYCSREDDYDGTPFAMDYWGQGTLVTVSSGGGGSGGGGSGGGGSGGGGSDIQMTQSPSSLSASVGDRVTITCHASQNINVWLSWYQQKPGKAPKLLIYKASNLHTGVPSRFTGSGSGTDFTFTISSLQPEDIATYYCQQGQTYPYTFGQGTKLEIK

SEQ ID NO: 67 - protein, 249 aa, Synthetic construct
DIQMTQSPSSLSASVGDRVTITCHASQNINVWLSWYQQKPGKAPKLLIYKASNLHTGVPSRFTGSGSGTDFTFTISSLQPEDIATYYCQQGQTYPYTFGQGTKLEIKGGGGSGGGGSGGGGSGGGGSQVQLVQSGAEVKKPGASVKLSCKASGYIFTRYYMYWVKQRPGQGLEWIGEINPNNGGTKFNEKFKNRATLTVDKSISTAYMELSRLRSDDTAVYYCSREDDYDGTPFAMDYWGQGTLVTVSS

SEQ ID NO: 68 - protein, 249 aa, Synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSGGGGSGGGGSGGGGSGGGGSEIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 69 - protein, 249 aa, Synthetic construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKGGGGSGGGGSGGGGSGGGGSEVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 70 - protein, 251 aa, Synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIK

SEQ ID NO: 71 - protein, 251 aa, Synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKGGGGSGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSS

SEQ ID NO: 72 - protein, 494 aa, Synthetic construct
QVQLVQSGAEVKKPGASVKLSCKASGYIFTRYYMYWVKQRPGQGLEWIGEINPNNGGTKFNEKFKNRATLTVDKSISTAYMELSRLRSDDTAVYYCSREDDYDGTPFAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGSSGSGSGSTGTSSSGTGTSAGTTGTSASTSGSGSGGGGGSGGGGSAGGDIQMTQSPSSLSASVGDRVTITCHASQNINVWLSWYQQKPGKAPKLLIYKASNLHTGVPSRFTGSGSGTDFTFTISSLQPEDIATYYCQQGQTYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 496 aa, Synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGSSGSGSGSTGTSSSGTGTSAGTTGTSASTSGSGSGGGGGSGGGGSAGGEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 494 aa, Synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGSSGSGSGSTGTSSSGTGTSAGTTGTSASTSGSGSGGGGGSGGGGSAGGEIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC